SEQUENCE LISTING (192 sequences)

SEQ ID NO: 1 - protein, 26 aa, Artificial
  FR1
QVQLQESGGGXVQAGGSLRLSCAASG

SEQ ID NO: 2 - protein, 14 aa, Artificial
  FR2
WXRQAPGKXXEXVA

SEQ ID NO: 3 - protein, 32 aa, Artificial
  FR3
RFTISRDNAKNTVYLQMNSLXXEDTAVYYCAA

SEQ ID NO: 4 - protein, 11 aa, Artificial
  FR4
XXQGTXVTVSS

SEQ ID NO: 5 - protein, 26 aa, Artificial
  FR1
QVQLQESGGGLVQAGGSLRLSCAASG

SEQ ID NO: 6 - protein, 14 aa, Artificial
  FR2
WFRQAPGKERELVA

SEQ ID NO: 7 - protein, 14 aa, Artificial
  FR2
WFRQAPGKEREFVA

SEQ ID NO: 8 - protein, 13 aa, Artificial
  FR2
WFRQAPGKEREGA

SEQ ID NO: 9 - protein, 14 aa, Artificial
  FR2
WFRQAPGKQRELVA

SEQ ID NO: 10 - protein, 14 aa, Artificial
  FR2
WFRQAPGKQREFVA

SEQ ID NO: 11 - protein, 13 aa, Artificial
  FR2
WYRQAPGKGLEWA

SEQ ID NO: 12 - protein, 32 aa, Artificial
  FR3
RFTISRDNAKNTVYLQMNSLKPEDTAVYYCAA

SEQ ID NO: 13 - protein, 11 aa, Artificial
  FR4
WGQGTQVTVSS

SEQ ID NO: 14 - protein, 11 aa, Artificial
  FR4
WGQGTLVTVSS

SEQ ID NO: 15 - protein, 5 aa, Artificial
  CDR1
SFGMS

SEQ ID NO: 16 - protein, 5 aa, Artificial
  CDR1
LNLMG

SEQ ID NO: 17 - protein, 5 aa, Artificial
  CDR1
INLLG

SEQ ID NO: 18 - protein, 5 aa, Artificial
  CDR1
NYWMY

SEQ ID NO: 19 - protein, 17 aa, Artificial
  CDR2
SISGSGSDTLYADSVKG

SEQ ID NO: 20 - protein, 16 aa, Artificial
  CDR2
TITVGDSTNYADSVKG

SEQ ID NO: 21 - protein, 16 aa, Artificial
  CDR2
TITVGDSTSYADSVKG

SEQ ID NO: 22 - protein, 17 aa, Artificial
  CDR2
SINGRGDDTRYADSVKG

SEQ ID NO: 23 - protein, 17 aa, Artificial
  CDR2
AISADSSTKNYADSVKG

SEQ ID NO: 24 - protein, 17 aa, Artificial
  CDR2
AISADSSDKRYADSVKG

SEQ ID NO: 25 - protein, 17 aa, Artificial
  CDR2
RISTGGGYSYYADSVKG

SEQ ID NO: 26 - protein, 13 aa, Artificial
  CDR3
DREAQVDTLDFDY

SEQ ID NO: 27 - protein, 6 aa, Artificial
  CDR3
GGSLSR

SEQ ID NO: 28 - protein, 8 aa, Artificial
  CDR3
RRTWHSEL

SEQ ID NO: 29 - protein, 7 aa, Artificial
  CDR3
GRSVSRS

SEQ ID NO: 30 - protein, 5 aa, Artificial
  CDR3
GRGSP

SEQ ID NO: 31 - protein, 17 aa, Artificial
  Myc Tag
AAAEQKLISEEDLNGAA

SEQ ID NO: 32 - protein, 30 aa, Artificial
  Linker
GGGGSGGGGSGGGGSGGGGSGGGGSGGGGS

SEQ ID NO: 33 - protein, 9 aa, Artificial
  Linker
GGGGSGGGS

SEQ ID NO: 34 - protein, 115 aa, Artificial [sequence omitted]

SEQ ID NO: 35 - protein, 115 aa, Artificial [sequence omitted]

SEQ ID NO: 36 - protein, 117 aa, Artificial
  FR2
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 37 - protein, 10 aa, Artificial
  CDR1
GGTFSSVGMG

SEQ ID NO: 38 - protein, 10 aa, Artificial
  CDR1
GFTFSNYGMI

SEQ ID NO: 39 - protein, 10 aa, Artificial
  CDR1
GGTFSSIGMG

SEQ ID NO: 40 - protein, 10 aa, Artificial
  CDR1
GFTFSNYWMY

SEQ ID NO: 41 - protein, 10 aa, Artificial
  CDR1
GFTLSSITMT

SEQ ID NO: 42 - protein, 10 aa, Artificial
  CDR1
GRTFSIYNMG

SEQ ID NO: 43 - protein, 10 aa, Artificial
  CDR1
GRTFTSYNMG

SEQ ID NO: 44 - protein, 10 aa, Artificial
  CDR1
GFTFSNYWMY

SEQ ID NO: 45 - protein, 10 aa, Artificial
  CDR1
GGTFSSIGMG

SEQ ID NO: 46 - protein, 10 aa, Artificial
  CDR1
GGIYRVNTVN

SEQ ID NO: 47 - protein, 10 aa, Artificial
  CDR1
GFTFSNYWMY

SEQ ID NO: 48 - protein, 10 aa, Artificial
  CDR
GFTLSSITMT

SEQ ID NO: 49 - protein, 17 aa, Artificial
  CDR2
AISRSGDSTYYAGSVKG

SEQ ID NO: 50 - protein, 17 aa, Artificial
  CDR2
GISDGGRSTSYADSVKG

SEQ ID NO: 51 - protein, 17 aa, Artificial
  CDR3
AISRSGDSTYYADSVKG

SEQ ID NO: 52 - protein, 17 aa, Artificial
  CDR3
TISPRAAVTYYADSVKG

SEQ ID NO: 53 - protein, 17 aa, Artificial
  CDR3
TINSGGDSTTYADSVKG

SEQ ID NO: 54 - protein, 17 aa, Artificial
  CDR3
TITRSGGSTYYADSVKG

SEQ ID NO: 55 - protein, 17 aa, Artificial
  CDR2
TISRSGGSTYYADSVKG

SEQ ID NO: 56 - protein, 17 aa, Artificial
  CDR2
TISPRAGSTYYADSVKG

SEQ ID NO: 57 - protein, 17 aa, Artificial
  CDR2
AISRSGDSTYYADSVKG

SEQ ID NO: 58 - protein, 16 aa, Artificial
  CDR2
TITRAGSTNYVESVKG

SEQ ID NO: 59 - protein, 17 aa, Artificial
  CDR2
TISPRAANTYYADSVKG

SEQ ID NO: 60 - protein, 17 aa, Artificial
  CDR2
TINSGGDSTTYADSVKG

SEQ ID NO: 61 - protein, 15 aa, Artificial
  CDR3
RPAGTPINIRRAYNY

SEQ ID NO: 62 - protein, 9 aa, Artificial
  CDR3
AYGRGTYDY

SEQ ID NO: 63 - protein, 15 aa, Artificial
  CDR3
RPAGTAINIRRSYNY

SEQ ID NO: 64 - protein, 15 aa, Artificial
  CDR3
SLKYWHRPQSSDFAS

SEQ ID NO: 65 - protein, 10 aa, Artificial
  CDR3
GTYYSRAYYR

SEQ ID NO: 66 - protein, 15 aa, Artificial
  CDR3
ARIGAAVNIPSEYDS

SEQ ID NO: 67 - protein, 15 aa, Artificial
  CDR3
RPAGTPINIRRAYNY

SEQ ID NO: 68 - protein, 15 aa, Artificial
  CDR3
SLIYKARPQSSDFVS

SEQ ID NO: 69 - protein, 15 aa, Artificial
  CDR3
RPAGTAINIRRSYNY

SEQ ID NO: 70 - protein, 13 aa, Artificial
  CDR3
NGRWRSWSSQRDY

SEQ ID NO: 71 - protein, 15 aa, Artificial
  CDR3
SLRYRDRPQSSDFLF

SEQ ID NO: 72 - protein, 10 aa, Artificial
  CDR3
GTYYSRAYYR

SEQ ID NO: 73 - protein, 124 aa, Artificial
  Nanobody
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSS

SEQ ID NO: 74 - protein, 118 aa, Artificial
  Nanobody
AVQLVESGGGLVQPGGSLRLSCAASGFTFSNYGMIWVRQAPGKGLERVSGISDGGRSTSYADSVKGRFTISRDNAKSTLYLRMNSLKPEDTAVYYCARAYGRGTYDYWGQGTQVTVSS

SEQ ID NO: 75 - protein, 124 aa, Artificial
  Nanobody
QVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 76 - protein, 124 aa, Artificial
  Nanobody
DVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSS

SEQ ID NO: 77 - protein, 119 aa, Artificial
  Nanobody
EVQLVESGGGLVQPGGSLRLSCAASGFTLSSITMTWVRQAPGKGLEWVSTINSGGDSTTYADSVKGRFTISRDNAKNTLYLQMNSLKPEDTAVYYCAKGTYYSRAYYRLRGGTQVTVSS

SEQ ID NO: 78 - protein, 124 aa, Artificial
  Nanobody
EVQLVESGGGLVQPGGSLRLSCAASGRTFSIYNMGWFRQAPGKEREFVATITRSGGSTYYADSVKGRFTISRDNAKNAVYMQMNSLKPEDTAVYYCAAARIGAAVNIPSEYDSWGQGTQVTVSS

SEQ ID NO: 79 - protein, 124 aa, Artificial
  Nanobody
EVQLVESGGGLVQPGGSLRLSCAASGRTFTSYNMGWFRQSPGKEREFVATISRSGGSTYYADSVKGRFTISRDSAKNAVYMQMNSLKPEDTAVYYCAAARIGAAVNIPSEYGSWGQGTQVTVSS

SEQ ID NO: 80 - protein, 124 aa, Artificial
  Nanobody
QVKLEESGGGLVQPGGSLRLSCAASGFTFSNYWMYWVRQAPGKGLEWVSTISPRAGSTYYADSVKGRFTISRDNAKNTLYLQMNSLEPDDTALYYCARSLIYKARPQSSDFVSWRQGTQVTVSS

SEQ ID NO: 81 - protein, 124 aa, Artificial
  Nanobody
AVQLVDSGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 82 - protein, 121 aa, Artificial
  Nanobody
AVQLVESGGGLVQPGGSLRLSCAASGGIYRVNTVNWYRQAPGLQRELVATITRAGSTNYVESVKGRFTISLDNAKNTMYLQMNSLKPDDTGVYYCNVNGRWRSWSSQRDYWGQGTQVTVSS

SEQ ID NO: 83 - protein, 124 aa, Artificial
  Nanobody
QVKLEESGGGLVQPGGSLRLSCAASGFTFSNYWMYWVRQAPGKGLEWVSTISPRAANTYYADSVKGRFTISRDNAKNTLYLQMNSLEPDDTALYYCAKSLRYRDRPQSSDFLFWRQGTQVTVSS

SEQ ID NO: 84 - protein, 119 aa, Artificial
  Nanobody
AVQLVESGGGLVQPGGSLRLSCAASGFTLSSITMTWVRQAPGKGLEWVSTINSGGDSTTYADSVKGRFTISRDNAKNTLYLQMNSLKPEDTAVYYCAKGTYYSRAYYRLRGGTQVTVSS

SEQ ID NO: 85 - protein, 124 aa, Artificial
  Nanobody
EVQLVESGGGLVQPGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSS

SEQ ID NO: 86 - protein, 124 aa, Artificial
  Nanobody
EVQLVESGGGLVQPGGSLRLSCAVSGGTFSSVGMGWFRQAPGKELEFVGAISRSGDSTYYAGSVKGRFTISRDGAKNTVYLQMNSLKDEDTAVYYCAARPAGTPINIRRAYNYWGQGTQVTVSS

SEQ ID NO: 87 - protein, 124 aa, Artificial
  Nanobody
EVQLVESGGGLVQPGGSLRLSCAVSGGTFSSVGMGWFRQAPGKGLEFVGAISRSGDSTYYAGSVKGRFTISRDGAKNTVYLQMNSLKDEDTAVYYCAARPAGTPINIRRAYNYWGQGTQVTVSS

SEQ ID NO: 88 - protein, 124 aa, Artificial
  Nanobody
EVQLVESGGGLVQPGGSLRLSCAVSGGTFSSVGMGWFRQAPGKGLEFVGAISRSGDSTYYAGSVKGRFTISRDGAKNTLYLQMNSLKDEDTAVYYCAARPAGTPINIRRAYNYWGQGTQVTVSS

SEQ ID NO: 89 - protein, 124 aa, Artificial
  Nanobody
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSS

SEQ ID NO: 90 - protein, 124 aa, Artificial
  Nanobody
EVQLVESGGGLVQPGGSLRLSCAVSGGTFSSVGMGWFRQAPGKGLEFVGAISRSGDSTYYAGSVKGRFTISRDGAKNSLYLQMNSLKTEDTAVYYCAARPAGTPINIRRAYNYWGQGTQVTVSS

SEQ ID NO: 91 - protein, 124 aa, Artificial
  Nanobody
EVQLVESGGGLVQPGGSLRLSCAVSGGTFSSVGMGWFRQAPGKGLEFVGAISRSGDSTYYAGSVKGRFTISRDGSKNSLYLQMNSLKTEDTAVYYCAARPAGTPINIRRAYNYWGQGTQVTVSS

SEQ ID NO: 92 - protein, 124 aa, Artificial
  Nanobody
EVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 93 - protein, 124 aa, Artificial
  Nanobody
EVQLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 94 - protein, 124 aa, Artificial
  Nanobody
EVQLVESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 95 - protein, 124 aa, Artificial
  Nanobody
EVQLVESGGGLVQPGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 96 - protein, 124 aa, Artificial
  Nanobody
EVQLVESGGGLVQPGGSLRLSCAVSGGTFSSIGMGWFRQAPGKGREFVGAISRSGDSTYYADSVKGRFTISRDGAKNTVYLQMNSLKDEDTAVYYCAGRPAGTAINIRRSYNYWGQGTQVTVSS

SEQ ID NO: 97 - protein, 124 aa, Artificial
  Nanobody
EVQLVESGGGLVQPGGSLRLSCAVSGGTFSSIGMGWFRQAPGKGLEFVGAISRSGDSTYYADSVKGRFTISRDGAKNTVYLQMNSLKDEDTAVYYCAGRPAGTAINIRRSYNYWGQGTQVTVSS

SEQ ID NO: 98 - protein, 124 aa, Artificial
  Nanobody
EVQLVESGGGLVQPGGSLRLSCAVSGGTFSSIGMGWFRQAPGKGLEFVGAISRSGDSTYYADSVKGRFTISRDGSKNTVYLQMNSLKDEDTAVYYCAGRPAGTAINIRRSYNYWGQGTQVTVSS

SEQ ID NO: 99 - protein, 124 aa, Artificial
  Nanobody
EVQLVESGGGLVQPGGSLRLSCAVSGGTFSSIGMGWFRQAPGKGLEFVGAISRSGDSTYYADSVKGRFTISRDGSKNSVYLQMNSLKDEDTAVYYCAGRPAGTAINIRRSYNYWGQGTQVTVSS

SEQ ID NO: 100 - protein, 124 aa, Artificial
  Nanobody
EVQLVESGGGLVQPGGSLRLSCAVSGGTFSSIGMGWFRQAPGKGLEFVGAISRSGDSTYYADSVKGRFTISRDGSKNSLYLQMNSLKDEDTAVYYCAGRPAGTAINIRRSYNYWGQGTQVTVSS

SEQ ID NO: 101 - protein, 124 aa, Artificial
  Nanobody
EVQLVESGGGLVQPGGSLRLSCAVSGGTFSSIGMGWFRQAPGKGLEFVGAISRSGDSTYYADSVKGRFTISRDGSKNSLYLQMNSLKTEDTAVYYCAGRPAGTAINIRRSYNYWGQGTQVTVSS

SEQ ID NO: 102 - protein, 124 aa, Artificial
  Nanobody
EVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSS

SEQ ID NO: 103 - protein, 124 aa, Artificial
  Nanobody
EVQLVESGGGLVQPGGSLRLSCAASGFTFSNYWMYWVRQAPGKGLEWVSTISPRAAVTYYADSVKGRFTISRDNSKNTLYLQMNSLEPDDTALYYCARSLKYWHRPQSSDFASWRRGTQVTVSS

SEQ ID NO: 104 - protein, 124 aa, Artificial
  Nanobody
EVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSS

SEQ ID NO: 105 - protein, 124 aa, Artificial
  Nanobody
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSS

SEQ ID NO: 106 - protein, 115 aa, Artificial
  Nanobody
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 107 - protein, 115 aa, Artificial
  Nanobody
QVQLQESGGGLVQPGNSLRLSCAASGFTFRNFGMSWVRQAPGKEPEWVSSISGSGSNTIYADSVKDRFTISRDNAKSTLYLQMNSLKPEDTAVYYCTIGGSLSRSSQGTQVTVSS

SEQ ID NO: 108 - protein, 114 aa, Artificial
  Nanobody
QVQLQESGGGLVQPGGSLRLTCTASGFTFSSFGMSWVRQAPGKGLEWVSAISSDSGTKNYADSVKGRFTISRDNAKKMLFLQMNSLRPEDTAVYYCVIGRGSPSSQGTQVTVSS

SEQ ID NO: 109 - protein, 114 aa, Artificial
  Nanobody
QVQLQESGGGLVQPGGSLRLTCTASGFTFRSFGMSWVRQAPGKGLEWVSAISADGSDKRYADSVKGRFTISRDNGKKMLTLDMNSLKPEDTAVYYCVIGRGSPASQGTQVTVSS

SEQ ID NO: 110 - protein, 121 aa, Artificial
  Nanobody
QVQLVESGGGLVQAGGSLRLSCAASGRAFIAYAMGWFRQGPGKEREFVAAISSYSGTNTNYADSVRGRFTISRDNVENMVYLQMNNLKPEDTAVYYCAADRRVLTSTSPFWGQGTQVTVSS

SEQ ID NO: 111 - protein, 112 aa, Artificial
  Nanobody
EVQLVESGGGLVQAGGSLRLSCAASGRTFSSYPMGWFRQASGKEREFVAAISRSGGSTYYEDFVKGRFTISRDNAKNTVYLQMNSLKPEDTAVYYCNVGKVWGQGTQVTVSS

SEQ ID NO: 112 - protein, 121 aa, Artificial
  Nanobody
QVKLEESGGGLVQAGGSLRLSCAASGRAFIAYAMGWFRQGPGKEREFVAAISSYSGTNTNYADSVRGRFTISRDNVENMVYLQMNNLKPEDTAVYYCAADRRVLTSTSPFWGQGTQVTVSS

SEQ ID NO: 113 - protein, 112 aa, Artificial
  Nanobody
QVKLEESGGGLVQAGDSLRLSCVASGRTFSRYAVGWFRQAPGKPREFVAAISRSGGSTYHEDSVRGRFTISRDNTGNTVYLQMNSLKPEDTAVYYCNVATYWGLGTQVTVSS

SEQ ID NO: 114 - protein, 125 aa, Artificial
  Nanobody
QVKLEESGGGLVQAGGSLRLSCAASGRTFDSYDMGWFRQAPGKERDFVAFISWTGGRTVYADSVKGRFTISRDNAKNTVYLQMNSLKPEDTALYYCAASKGAWPLYSLSSRYDYWGQGTQVTVSS

SEQ ID NO: 115 - protein, 121 aa, Artificial
  Nanobody
EVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 116 - protein, 112 aa, Artificial
  Nanobody
EVQLLESGGGLVQPGGSLRLSCAASGRTFSSYPMGWFRQAPGKGLEFVSAISRSGGSTYYEDFVKGRFTISRDNSKNTLYLQMNSLRAEDTAVYYCNVGKVWGQGTLVTVSS

SEQ ID NO: 117 - protein, 242 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 118 - protein, 242 aa, Artificial
  Nanobody construct
QVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 119 - protein, 237 aa, Artificial
  Nanobody construct
EVQLVESGGGLVQPGGSLRLSCAASGFTLSSITMTWVRQAPGKGLEWVSTINSGGDSTTYADSVKGRFTISRDNAKNTLYLQMNSLKPEDTAVYYCAKGTYYSRAYYRLRGGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 120 - protein, 242 aa, Artificial
  Nanbody construct
DVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 121 - protein, 236 aa, Artificial
  Nanbody construct
AVQLVESGGGLVQPGGSLRLSCAASGFTFSNYGMIWVRQAPGKGLERVSGISDGGRSTSYADSVKGRFTISRDNAKSTLYLRMNSLKPEDTAVYYCARAYGRGTYDYWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 122 - protein, 242 aa, Artificial
  Nanobody construct
EVQLVESGGGLVQPGGSLRLSCAASGRTFSIYNMGWFRQAPGKEREFVATITRSGGSTYYADSVKGRFTISRDNAKNAVYMQMNSLKPEDTAVYYCAAARIGAAVNIPSEYDSWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 123 - protein, 242 aa, Artificial
  Nanobody construct
EVQLVESGGGLVQPGGSLRLSCAASGRTFTSYNMGWFRQSPGKEREFVATISRSGGSTYYADSVKGRFTISRDSAKNAVYMQMNSLKPEDTAVYYCAAARIGAAVNIPSEYGSWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 124 - protein, 239 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 125 - protein, 239 aa, Artificial
  Nanobody construct
QVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 126 - protein, 234 aa, Artificial
  Nanobody construct
EVQLVESGGGLVQPGGSLRLSCAASGFTLSSITMTWVRQAPGKGLEWVSTINSGGDSTTYADSVKGRFTISRDNAKNTLYLQMNSLKPEDTAVYYCAKGTYYSRAYYRLRGGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 127 - protein, 239 aa, Artificial
  Nanobody construct
DVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 128 - protein, 233 aa, Artificial
  Nanobody construct
AVQLVESGGGLVQPGGSLRLSCAASGFTFSNYGMIWVRQAPGKGLERVSGISDGGRSTSYADSVKGRFTISRDNAKSTLYLRMNSLKPEDTAVYYCARAYGRGTYDYWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 129 - protein, 239 aa, Artificial
  Nanobody construct
EVQLVESGGGLVQPGGSLRLSCAASGRTFSIYNMGWFRQAPGKEREFVATITRSGGSTYYADSVKGRFTISRDNAKNAVYMQMNSLKPEDTAVYYCAAARIGAAVNIPSEYDSWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 130 - protein, 239 aa, Artificial
  Nanobody construct
EVQLVESGGGLVQPGGSLRLSCAASGRTFTSYNMGWFRQSPGKEREFVATISRSGGSTYYADSVKGRFTISRDSAKNAVYMQMNSLKPEDTAVYYCAAARIGAAVNIPSEYGSWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 131 - protein, 248 aa, Artificial
  Nanobody construct
AVQLVDSGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSGGGGSGGGSAVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSS

SEQ ID NO: 132 - protein, 248 aa, Artificial
  Nanobody construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSAVQLVDSGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 133 - protein, 248 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTVYLQMNSLKDEDTAVYYCAARPAGTPINIRRAYNYWGQGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 134 - protein, 248 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 135 - protein, 248 aa, Artificial
  Nanobody construct
EVQLVESGGGLVQPGGSLRLSCAVSGGTFSSVGMGWFRQAPGKGLEFVGAISRSGDSTYYAGSVKGRFTISRDGSKNSLYLQMNSLKTEDTAVYYCAARPAGTPINIRRAYNYWGQGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 136 - protein, 248 aa, Artificial
  Nanobody construct
EVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 137 - protein, 248 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNTLYLQMNSLEPDDTALYYCARSLKYWHRPQSSDFASWRRGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 138 - protein, 248 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 139 - protein, 248 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 140 - protein, 248 aa, Artificial
  Nanobody construct
EVQLVESGGGLVQPGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 141 - protein, 248 aa, Artificial
  Nanobody construct
EVQLVESGGGLVQPGGSLRLSCAVSGGTFSSIGMGWFRQAPGKGLEFVGAISRSGDSTYYADSVKGRFTISRDGSKNSLYLQMNSLKTEDTAVYYCAGRPAGTAINIRRSYNYWGQGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 142 - protein, 245 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTVYLQMNSLKDEDTAVYYCAARPAGTPINIRRAYNYWGQGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 143 - protein, 245 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 144 - protein, 245 aa, Artificial
  Nanobody construct
EVQLVESGGGLVQPGGSLRLSCAVSGGTFSSVGMGWFRQAPGKGLEFVGAISRSGDSTYYAGSVKGRFTISRDGSKNSLYLQMNSLKTEDTAVYYCAARPAGTPINIRRAYNYWGQGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 145 - protein, 245 aa, Artificial
  Nanobody construct
EVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 146 - protein, 245 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNTLYLQMNSLEPDDTALYYCARSLKYWHRPQSSDFASWRRGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 147 - protein, 245 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 148 - protein, 245 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 149 - protein, 245 aa, Artificial
  Nanobody construct
EVQLVESGGGLVQPGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 150 - protein, 245 aa, Artificial
  Nanobody construct
EVQLVESGGGLVQPGGSLRLSCAVSGGTFSSIGMGWFRQAPGKGLEFVGAISRSGDSTYYADSVKGRFTISRDGSKNSLYLQMNSLKTEDTAVYYCAGRPAGTAINIRRSYNYWGQGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 151 - protein, 251 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAQVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 152 - protein, 246 aa, Artificial
  Nanobody construct
EVQLVESGGGLVQPGGSLRLSCAASGFTLSSITMTWVRQAPGKGLEWVSTINSGGDSTTYADSVKGRFTISRDNAKNTLYLQMNSLKPEDTAVYYCAKGTYYSRAYYRLRGGTQVTVSSAAADVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSS

SEQ ID NO: 153 - protein, 245 aa, Artificial
  Nanobody construct
AVQLVESGGGLVQPGGSLRLSCAASGFTFSNYGMIWVRQAPGKGLERVSGISDGGRSTSYADSVKGRFTISRDNAKSTLYLRMNSLKPEDTAVYYCARAYGRGTYDYWGQGTQVTVSSAAAEVQLVESGGGLVQPGGSLRLSCAASGRTFTSYNMGWFRQSPGKEREFVATISRSGGSTYYADSVKGRFTISRDSAKNAVYMQMNSLKPEDTAVYYCAAARIGAAVNIPSEYGSWGQGTQVTVSS

SEQ ID NO: 154 - protein, 251 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAEVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSS

SEQ ID NO: 155 - protein, 248 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSQVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 156 - protein, 243 aa, Artificial
  Nanobody construct
EVQLVESGGGLVQPGGSLRLSCAASGFTLSSITMTWVRQAPGKGLEWVSTINSGGDSTTYADSVKGRFTISRDNAKNTLYLQMNSLKPEDTAVYYCAKGTYYSRAYYRLRGGTQVTVSSDVQLVESGGGLVQPGGSLRLSCAASGFTFSNYWMYWVRQAPGKGLEWVSTISPRAAVTYYADSVKGRFTISRDNAKNTLYLQMNSLEPDDTALYYCARSLKYWHRPQSSDFASWRRGTQVTVSS

SEQ ID NO: 157 - protein, 242 aa, Artificial
  Nanobody construct
AVQLVESGGGLVQPGGSLRLSCAASGFTFSNYGMIWVRQAPGKGLERVSGISDGGRSTSYADSVKGRFTISRDNAKSTLYLRMNSLKPEDTAVYYCARAYGRGTYDYWGQGTQVTVSSEVQLVESGGGLVQPGGSLRLSCAASGRTFTSYNMGWFRQSPGKEREFVATISRSGGSTYYADSVKGRFTISRDSAKNAVYMQMNSLKPEDTAVYYCAAARIGAAVNIPSEYGSWGQGTQVTVSS

SEQ ID NO: 158 - protein, 248 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSEVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSS

SEQ ID NO: 159 - protein, 251 aa, Artificial
  Nanobody construct
EVQLVESGGGLVQPGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAEVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 160 - protein, 251 aa, Artificial
  Nanobody construct
EVQLVESGGGLVQPGGSLRLSCAVSGGTFSSVGMGWFRQAPGKGLEFVGAISRSGDSTYYAGSVKGRFTISRDGSKNSLYLQMNSLKTEDTAVYYCAARPAGTPINIRRAYNYWGQGTQVTVSSAAAEVQLVESGGGLVQPGGSLRLSCAVSGGTFSSIGMGWFRQAPGKGLEFVGAISRSGDSTYYADSVKGRFTISRDGSKNSLYLQMNSLKTEDTAVYYCAGRPAGTAINIRRSYNYWGQGTQVTVSS

SEQ ID NO: 161 - protein, 251 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSSAAAEVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSS

SEQ ID NO: 162 - protein, 251 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSAAAEVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSS

SEQ ID NO: 163 - protein, 248 aa, Artificial
  Nanobody construct
EVQLVESGGGLVQPGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSEVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSS

SEQ ID NO: 164 - protein, 248 aa, Artificial
  Nanobody construct
EVQLVESGGGLVQPGGSLRLSCAVSGGTFSSVGMGWFRQAPGKGLEFVGAISRSGDSTYYAGSVKGRFTISRDGSKNSLYLQMNSLKTEDTAVYYCAARPAGTPINIRRAYNYWGQGTQVTVSSEVQLVESGGGLVQPGGSLRLSCAVSGGTFSSIGMGWFRQAPGKGLEFVGAISRSGDSTYYADSVKGRFTISRDGSKNSLYLQMNSLKTEDTAVYYCAGRPAGTAINIRRSYNYWGQGTQVTVSS

SEQ ID NO: 165 - protein, 248 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSSEVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSS

SEQ ID NO: 166 - protein, 248 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSEVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSS

SEQ ID NO: 167 - protein, 369 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAQVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 168 - protein, 369 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAEVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSAAAQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 169 - protein, 363 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSQVKLEESGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 170 - protein, 363 aa, Artificial
  Nanobody construct
EVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSEVQLVESGGGLVQAGGSLRLSCAVSGGTFSSVGMGWFRQAPGKEREFVGAISRSGDSTYYAGSVKGRFTISRDGAKNTVYLQMNSLKDEDTAVYYCAARPAGTPINIRRAYNYWGQGTQVTVSSQVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 171 - protein, 375 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSSAAAEVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 172 - protein, 375 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSAAAEVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSAAAEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 173 - protein, 369 aa, Artificial
  Nanobody construct
EVQLVESGGGLVQPGGSLRLSCAVSGGTFSSVGMGWFRQAPGKGLEFVGAISRSGDSTYYAGSVKGRFTISRDGAKNTLYLQMNSLKTEDTAVYYCAARPAGTPINIRRAYNYWGQGTQVTVSSEVQLVESGGGLVQPGGSLRLSCAASGFTFSNYWMYWVRQAPGKGLEWVSTISPRAAVTYYADSVKGRFTISRDNSKNSLYLQMNSLEPDDTALYYCARSLKYWHRPQSSDFASWRRGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 174 - protein, 369 aa, Artificial
  Nanobody construct
EVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSEVQLVESGGGLVQPGGSLRLSCAASGFTFSNYWMYWVRQAPGKGLEWVSTISPRAAVTYYADSVKGRFTISRDNSKNSLYLQMNSLETDDTALYYCARSLKYWHRPQSSDFASWRRGTQVTVSSEVQLLESGGGLVQPGGSLRLSCAASGRAFIAYAMGWFRQAPGKGLEFVSAISSYSGTNTNYADSVRGRFTISRDNSKNTLYLQMNSLRAEDTAVYYCAADRRVLTSTSPFWGQGTLVTVSS

SEQ ID NO: 175 - protein, 258 aa, Artificial
  Nanobody construct
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSSGGGGSGGGSQVKLEESGGGLVQPGGSLRLSCAASGFTFSNYWMYWVRQAPGKGLEWVSTISPRAGSTYYADSVKGRFTISRDNAKNTLYLQMNSLEPDDTALYYCARSLIYKARPQSSDFVSWRQGTQVTVSS

SEQ ID NO: 176 - protein, 258 aa, Artificial
  Nanobody construct
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSSGGGGSGGGSQVKLEESGGGLVQPGGSLRLSCAASGFTFSNYWMYWVRQAPGKGLEWVSTISPRAANTYYADSVKGRFTISRDNAKNTLYLQMNSLEPDDTALYYCAKSLRYRDRPQSSDFLFWRQGTQVTVSS

SEQ ID NO: 177 - protein, 258 aa, Artificial
  Nanobody construct
AVQLVDSGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSGGGGSGGGSEVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSS

SEQ ID NO: 178 - protein, 255 aa, Artificial
  Nanobody construct
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGGSGGGSQVKLEESGGGLVQPGGSLRLSCAASGFTFSNYWMYWVRQAPGKGLEWVSTISPRAANTYYADSVKGRFTISRDNAKNTLYLQMNSLEPDDTALYYCAKSLRYRDRPQSSDFLFWRQGTQVTVSS

SEQ ID NO: 179 - protein, 382 aa, Artificial
  Nanobody construct
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSSGGGGSGGGSAVQLVDSGGGLVQAGGSLRLSCAVSGGTFSSIGMGWFRQAPGKEREFVGAISRSGDSTYYADSVKGRFTISRDGAKNTVYLQMNSLKDEDTAVYYCAGRPAGTAINIRRSYNYWGQGTQVTVSSGGGGSGGGSAVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSS

SEQ ID NO: 180 - protein, 382 aa, Artificial
  Nanobody construct
AVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSSGGGGSGGGSQVKLEESGGGLVQPGGSLRLSCAASGFTFSNYWMYWVRQAPGKGLEWVSTISPRAGSTYYADSVKGRFTISRDNAKNTLYLQMNSLEPDDTALYYCARSLIYKARPQSSDFVSWRQGTQVTVSS

SEQ ID NO: 181 - protein, 382 aa, Artificial
  Nanobody construct
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVKLEESGGGLVQPGGSLRLSCAASGFTFSNYWMYWVRQAPGKGLEWVSTISPRAANTYYADSVKGRFTISRDNAKNTLYLQMNSLEPDDTALYYCAKSLRYRDRPQSSDFLFWRQGTQVTVSS

SEQ ID NO: 182 - protein, 382 aa, Artificial
  Nanobody construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVKLEESGGGLVQPGGSLRLSCAASGFTFSNYWMYWVRQAPGKGLEWVSTISPRAGSTYYADSVKGRFTISRDNAKNTLYLQMNSLEPDDTALYYCARSLIYKARPQSSDFVSWRQGTQVTVSSGGGGSGGGSEVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSS

SEQ ID NO: 183 - protein, 379 aa, Artificial
  Nanobody construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGGSGGGSQVKLEESGGGLVQPGGSLRLSCAASGFTFSNYWMYWVRQAPGKGLEWVSTISPRAANTYYADSVKGRFTISRDNAKNTLYLQMNSLEPDDTALYYCAKSLRYRDRPQSSDFLFWRQGTQVTVSS

SEQ ID NO: 184 - protein, 379 aa, Artificial; identical to SEQ ID NO: 183
  Nanobody construct

SEQ ID NO: 185 - protein, 12 aa, Artificial
  Linker
EPKTPKPQPAAA

SEQ ID NO: 186 - protein, 15 aa, Artificial
  Linker
GGGGSGGGGSGGGGS

SEQ ID NO: 187 - protein, 7 aa, Artificial
  Linker
SGGSGGS

SEQ ID NO: 188 - protein, 40 aa, Artificial
  A-BETA protein
DAEFRHDSGYEVHHQKLVFFAEDVGSNKGAIIGLMVGGVV

SEQ ID NO: 189 - protein, 42 aa, Artificial [sequence omitted]

SEQ ID NO: 190 - protein, 125 aa, Artificial
  Nanobody
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSS

SEQ ID NO: 191 - protein, 122 aa, Artificial
  Nanobody
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 192 - protein, 10 aa, Artificial
  Linker
GGGGSGAGGA

(19) United States
(12) Patent Application Publication
(10) Pub. No.: US 2008/0107601 A1
(43) Pub. Date: May 8, 2008

(54) Nanobodies Tm Against Amyloid-Beta and Polypeptides Comprising the Same for the Treatment of Degenerative Neural Diseases Such as Alzheimer's Disease

(75) Inventors: Marc Lauwereys, Haaltert (BE); Fred Van Leuven, Linden (BE); Ingrid Van Der Auwera, Langdorp (BE); Stefaan Wera, Bierbeek (BE); Pascal Merchiers, Kasterlee (BE)

Correspondence Address: WOLF GREENFIELD & SACKS, P.C., 600 ATLANTIC AVENUE, BOSTON, MA, 02210-2206, US

(73) Assignee: Ablynx N.V., Zwijnaarde (BE)

(21) Appl. No.: 11/665,356

(22) PCT Filed: Oct. 13, 2005
(86) PCT No.: PCT/EP05/11018

§ 371 Date: Oct. 25, 2007

Related U.S. Application Data
(60) Provisional application No. 60/618,148, filed on Oct. 13, 2004.
(60) Provisional application No. 60/718,617, filed on Sep. 20, 2005.

(51) Int. Cl. A61K 39/395; A61K 49/00; C07H 21/04; C07K 16/18; C07K 2/00; C12P 21/00; G01N 33/53

(52) U.S. Cl. 424/9.1; 424/130.1; 435/69.1; 435/7.1; 530/300; 530/387.1; 536/23.5

(57) ABSTRACT

The present invention relates to anti-A-beta polypeptides comprising at least one Nanobody, or a functional fragment thereof, directed against A-beta, for the treatment of diseases or disorders mediated by A-beta or dysfunction thereof, or mediated by amyloid plaque formation.

The present invention relates to Nanobodies™ against amyloid-beta (herein also referred to an “A-beta”, as “Beta-amyloid peptide/protein” or as “Beta-AP”), as well as to polypeptides that comprise or essentially consist of one or more Nanobodies against A-beta. [Note: Nanobody™, Nanobodies™ and Nanoclone™ are trademarks of Ablynx N. V]

The invention also relates to nucleic acids encoding such Nanobodies and polypeptides; to methods for preparing such Nanobodies and polypeptides; to host cells expressing or capable of expressing such Nanobodies or polypeptides; to compositions, and in particular to pharmaceutical compositions, that comprise such Nanobodies, polypeptides, nucleic acids and/or host cells; and to uses of such Nanobodies, polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

Weksler M, Immunity and Ageing, 2004, 1:2, which was published after the priority date of the present application, provides a review of the current methodology and techniques for the immunotherapy of Alzheimer's disease.

Animal models of AD and other neurodegenerative diseases are known in the art. One example is the APP transgenic mouse model described by Games et al., Nature, 1995, 373:523-527.

Several degenerative neural diseases are caused by the improper folding or processing of proteins or by prions, both of which result in invasive neural depositions known as amyloid plaques. The most widely known degenerative neural disease is probably Alzheimer's Disease (AD). Examples of other neurogenerative diseases and disorders will b clear to the skilled person.

The incidence of AD warrants an urgent and umnet medical need: between 10 and 40% of all people aged 65 to 85 develop AD. Moreover, this segment of the population continues to grow exponentially. Therefore, from a humane, as well as from a social and economical point of view, it is imperative to find ways to efficiently diagnose and treat this devastating disorder. Concerning treatment, drugs are needed not only to slow or stop the disease progression, but also to restore brain damage that has already occurred during the initial stages of AD (before diagnosis). At this moment, neither early-diagnosis nor therapy treatment are efficient.

AD is defined as a dementia that coincides with the presence in the brain of extracellular amyloid plaques, composed mainly of amyloid peptides, and by intracellular neurofibrillary tangles (NFT) composed mainly of protein tau.

A primary component of amyloid plaques is beta amyloid peptide (beta-AP), a highly insoluble peptide 39-43 amino acids in length that has a strong propensity to adopt beta sheet structures, oligomerize and form protein aggregates. Production of beta-AP occurs when amyloid polypeptide precursor is cleaved by certain proteases, a group known as secretases. Cleavage by beta-secretase at the amino terminus of beta amyloid peptide and cleavage by gamma-secretase between residues 39 and 43 (most often at residue 42) constitute the means by which this peptide is produced. Cleavage by alpha-secretase (and other metalloproteases) affords a soluble cleavage product by cleaving between residues 16 and 17 of the beta amyloid peptide. This pathway reduces the potential accumulation of beta-AP by producing a soluble product.

A-beta protein is the principal component of the senile plaques characteristic of Alzheimer's disease (AD). A-beta is produced from the A-beta precursor protein (APP) by two proteolytic events. A beta-secretase activity cleaves APP at the N terminus of A-beta (beta-site) between amino acids Met-671 and Asp-672 (using the numbering of the 770-aa isoform of APP). Cleavage at the beta-site yields a membrane-associated APP fragment of 99 aa (C99). A second site within the transmembrane domain of C99 (gamma site) can then be cleaved by a gamma-secretase to release A-beta, a peptide of 39-42 aa. APP can alternatively be cleaved within its A-beta region, predominately at the alpha-secretase cleavage site of APP, to produce a C-terminal APP fragment of 83 aa (C83), which can also be further cleaved by gamma-secretase to produce a small secreted peptide, p3. APP is closely related to APLP1 and APLP2 (termed APLP or APP-like proteins).

The intra- and extracellular A-beta adopts a P-sheet conformation and forms intermediate named ADDL (amyloid derived diffusible ligands) and protofibrils, finally precipitates in the form of amyloid fibrils which assemble into amyloid plaques. In these processes, the more hydrophobic A-beta-42 peptide is presumed to serve as a nucleating agent around which the plaques steadily grow.

A number of missense mutations in APP have been implicated in forms of early-onset familial AD. All of these are at or near one of the canonical cleavage sites of APP. Thus, the Swedish double mutation (K670N/M671L) is immediately adjacent to the beta-cleavage site and increases the efficiency of beta-secretase activity, resulting in more total A-beta. Any of three mutations at APP residue 717, near the gamma site, increases the proportion of a more amyloidogenic 42-aa form of A-beta [A-beta (1-42)] relative to the more common 40-residue form LA-beta (1-40)].

Two additional mutations of APP have been described which are close but not adjacent to the alpha-site. A mutation (A692G, A-beta residue 21) in a Flemish family and a mutation (E693Q, A-beta residue 22) in a Dutch family each have been implicated in distinct forms of familial AD. The Flemish mutation, in particular, presents as a syndrome of repetitive intracerebral hemorrhages or as an AD-type dementia. The neuropathological findings include senile plaques in the cortex and hippocampus, and usually multiple amyloid deposits in the walls of cerebral microvessels.

Recently, a membrane-associated aspartyl protease, BACE (also called beta-secretase or Asp2) has been shown to exhibit properties expected of a beta-secretase. This enzyme cleaves APP at its beta-site and between Tyr-10 and Glu-11 of the A-beta region with comparable efficiency. A-beta fragments cleaved at this latter site have been observed in amyloid plaques in AD and in media of APP-transfected HEK293 human embryonic kidney cells. Several groups also observed the presence in the database of an additional aspartyl protease, BACE2 (also called Asp1), a close homologue of BACE (hereafter referred to as BACE1).

BACE2 cleaves APP at its beta-site and more efficiently at sites within the A-beta region of APP, after Phe-19 and Phe-20 of A-beta. These internal A-beta-sites are adjacent to the Flemish APP mutation at residue 21, and this mutation markedly increases the proportion of beta-site cleavage product generated by BACE2. Conservative beta-site mutations of APP that either increase (the Swedish mutation) or inhibit (M671V) beta-secretase activity affect BACE1 and BACE2 activity similarly. BACE2, like BACE1, proteolyzes APP maximally at acidic pH. Moreover, alteration of a single Arg common to both enzymes blocks their ability to cleave at the beta-site of APP but not at their respective sites internal to A-beta. The identification of distinct BACE1 and BACE2 specificities and a key active-site residue important for beta-site cleavage may suggest strategies for selectively inhibiting beta-secretase activity. BACE2 cleavage of wild-type APP within the A-beta region can limit production of intact A-beta in BACE2-expressing tissues.

So like BACE1, BACE2 efficiently cleaves sites internal to the A-beta region of APP. Although both enzymes cleave within A-beta, the fragments of A-beta produced by these internal cleavages may have different clinical consequences. BACE1-generated A-beta fragments beginning at Glu-11 of A-beta have been observed in senile plaques, and fragments of this size have been shown to be more amyloidogenic and more neurotoxic than full-length A-beta. It may also be important that the BACE1-generated A-beta fragments, like full-length A-beta, include the HHQK sulfate-binding region of A-beta, which can associate with sulfated proteoglycans found in senile plaques. In contrast, BACE2-cleaved internal fragments (starting at A-beta Phe-19 and Phe-20) lack the HHQK domain and have not to date been observed in senile plaques. Moreover, fragments of the size of p3 (starting at A-beta Leu-17) or smaller appear to be less amyloidogenic and neurotoxic in tissue culture. BACE2 is more efficient at cleaving within A-beta than BACE1 and less efficient at generating C99. Furthermore it is demonstrated that BACE2 can efficiently degrade C99. These observations imply that BACE2 might limit the production of pathogenic forms of A-beta (i.e., fragments beginning at Asp-1 or Glu-11) in cells that express both BACE1 and BACE2.

Protein tau is a cytosolic, microtubule-binding protein whose affinity for microtubules is regulated by phosphorylation. Hyper-phosphorylated tau is found in the brain of AD patients as paired helical filaments (PHF-tau). PHF-tau forms even in vitro. PHF-tau has reduced affinity for binding to microtubules, and is thought to be the initial and major component of the NFT. Mutations in the gene encoding tau lead to another type of dementia, i.e. Frontotemporal Dementia with Parkinsonism-17 (FTDP-17), but not to AD.

Tau is a microtubule-associated protein that stabilizes the neuronal cytoskeleton and participates in vesicular transport and axonal polarity. In the brain, there are six isoforms of tau, produced by alternative mRNA splicing of a single gene located on chromosome 17. Pathological alterations in tau occur in several neurodegenerative disorders, including Alzheimer disease, supranuclear palsy, and frontotemporal dementia with parkinsonism.

In AD, insoluble neurofibrillary tangles (NFTs) composed of hyperphosphorylated forms of tau accumulate initially within the entorhinal cortex and CA1 subfield of the hippocampus. Recent studies have begun to clarify the sequence of tau alterations that lead to neurodegeneration, including conformational changes and hyperphosphorylation. An aberrant folded conformational change in tau appears to be one of the earliest tau pathological events. Such alterations in tau may reduce its binding affinity for microtubules, thereby leading to depolymerization of microtubules and contributing to the neuronal loss observed in AD.

Caspases are cysteine aspartate proteases that are critically involved in apoptosis. These enzymes can be broadly divided into initiator and executioner caspases, with the former functioning to initiate apoptosis by activating executioner caspases and the latter acting on downstream effector substrates that result in the progression of apoptosis and the appearance of hallmark morphological changes such as cell shrinkage, nuclear fragmentation, and membrane blebbing. Increasing evidence suggests that caspases are activated in the AD brain. Furthermore, components of the neuronal cytoskeleton, including tau, are targeted by caspases following apoptotic stimuli. Recent evidence now implicates the caspase-cleavage of tau in tangle pathology.

A recent study (Rissman et al., J. Clin. Invest., 114(1), 121-130, 2004) suggests that caspase-cleavage of tau is an early event in tangle formation in AD. Caspase-cleaved tau catalyzes filament formation adopts a conformation found in early-stage tangles, and can be hyperphosphorylated. Caspase-cleavage of tau also colocalizes with A-beta and developing tangles in both transgenic mice and the AD brain. In primary cortical neurons, A-beta-induced caspase activation leads to tau cleavage and generates tangle-like morphology. This suggests that caspase activation is an early event in NFT formation that can be triggered by A-beta, and that caspase activation may contribute to an important hallmark lesion of AD. Both intracellular and extracellular A-beta may induce caspase-cleavage of tau.

Hyperphosphorylation of tau is the prevailing hypothesis in the development of tangle pathology, since hyperphosphorylation can promote PHF self-assembly. It has been demonstrated that tau can be hyperphosphorylated after caspase-cleavage, therefore suggesting that production of tau does not preclude subsequent hyperphosphorylation.

Mutations in the APP gene, or in PS1 (“gamma-secretase”) cause early-onset familial AD. Examples of APP mutations are the ‘Swedish’ and ‘London’ mutations located respectively near the β- and gamma-secretase cleavage sites. These mutations increase the formation of A-beta peptides and especially of A-beta-42, and thereby increase the formation of amyloid aggregates and plaques. Whereas initially plaques were believed to be a major trigger for the development of AD, current studies emphasize the role of protofibrils and ADDL as the major toxic components (Walsh et al. (2002) Nature 416, 535-539; Lambert et al. (1998) Proc. Natl. Acad. Sci. USA 95, 6448-6453; Dewachter and Van Leuven, Lancet Neurology, 1(7), 409-416, 2002). It is even conceivable that plaques are a mechanism whereby the neurotoxic peptides are actually rendered biologically inactive.

A recent study demonstrated that the clearance of amyloid also resulted in the removal of early-stage tau pathology in mice that develop both amyloid plaques and neurofibrillary tangles (Oddo et al. (2004) Neuron 43, 321-332). Anti-tangle antibodies removed early tangles but not the plaque, and had no impact on advanced tangles.

Most current treatments of AD target the acetylcholine deficiency (reviewed by Auld et al. (2002) Progress in Neurobiology 68, 209-245) using acetylcholinesterase inhibitors (marketed as Reminyl of J&J, Exelon of Novartis, Aricept of Pfizer). The acetylcholine deficit reflects the degeneration of cholinergic neurons of the basal forebrain and appears to correlate well with the neuropsychiatric manifestations of the disease. Therefore treatment with acetylcholinesterase inhibitors has some beneficial effects but cannot cure or stop the progression of the disease, as the etiology of the neurodegeneration is left untreated.

Memantine is an NMDA receptor antagonist (Merz Pharmaceuticals) that appears to slow down cognitive deterioration and to delay progression in AD patients with moderate to severe cognitive impairment (Phase III clinical trials, Reisberg et al (2003) N Engl. J. Med. 348, 1333-1341). Although this drug represents a novel type and even promising therapy for the short-term or near future, it remains also a symptomatic therapy and neither cures nor stops the progression of the disease.

Some current experimental therapeutic strategies focus on A-beta as target. There are 3 major research lines:
a) the development of small molecules (often peptido-mimetics) named beta-sheet breakers, which are designed to interfere with the beta-sheet structure of amyloid peptide aggregates. It has been demonstrated that a stable “beta-sheet breaker”, when administered to a transgenic mouse model of AD, is able to penetrate the blood brain barrier and reduce the number of plaques (Permanne et al. (2002) FASEB J. 16, 860-862). It remains to be demonstrated whether this approach results in cognitive protection and/or restoration. Given the toxicity of soluble protofibrillar forms of AD, the efficient dissolution of amyloid plaques and the concomitant increase in soluble small aggregates might even worsen the neurodegeneration.
b) the development of small molecules which inhibit the proteolytic processing of APP into amyloid peptides. Inhibitors of the beta- or gamma-secretase should efficiently block the formation of A-beta and hence protect the brain from neurotoxic effects of amyloid. Best studied inhibitor is the gamma-secretase inhibitor DAPT whose administration reduces brain A-beta levels in young animals and CSF. It also reduces A-beta levels in plasma—but not brain—in older (plaque-containing) animals (Lanz et al. (2003) J. Pharmacol. Exp. Ther. in press; Dovey et al. (2001) J. Neurochem. 76, 173-181). A central question remains regarding the toxicity of these agents since gamma-secretase is involved in many cellular processes such as Notch-signalling (Francis et al (2002) Dev. Cell 3, 85-97). Furthermore, a knock-out of the PS1 gene, encoding the essential subunit of gamma-secretase, is lethal. On the other hand, mice with a “neuron specific knock-out” of PS1 are viable and have markedly reduced A-beta levels that prevents plaque formation. Nevertheless, this did not prevent cognitive defects, and even aggravated them; an explanation for this may be the accumulation of neurotoxic C-terminal fiagments of APP (β-CTF or C99) which are the immediate precursor of A-beta, and contain the entire amyloid sequence (Dewachter et al, J. Neurosci., 22(9), 3445-53, 2002).
c) Passive and active vaccination against A-beta. This research line started with the observation (Schenk et al. (1999) Nature 400, 173-177) that vaccination of transgenic AD mice with A-beta-42 prevented the formation of amyloid plaques. In a first experiment, monthly vaccination of young adult mice (age 6 weeks) essentially prevented plaque formation and the concomitant inflammatory reaction in the brain, i.e. absence of amyloid plaques, of astrocytosis and microgliosis. Vaccination starting at a later age, when amyloid plaques were already established, resulted in a partial clearance. Subsequently, other groups independently demonstrated that vaccination with A-beta improved the behavioral and memory deficits as measured in the water maze memory tests (Janus et al. (2000) Nature 408, 979-982; Morgan et al (2000) Nature 408, 982-985).

Given the side-effects of vaccination with the entire A-beta, alternative shorter peptides have been designed and successfully used to vaccinate transgenic mice, i.e. K6-A-beta-1-30 (Sigurdsson et al. (2001) Am. J. Pathol. 159, 439-447) and A-beta-4-10 (McLaurin et al., Nat. Med., 8(11), 1263-69, (2002)) and even bacteriophages expressing the A-beta-3-6 sequence as the EFRH epitope (Frenkel et al. (2003) Proc. Natl. Acad. Sci. USA 97, 11455-11459).

Following the promising pre-clinical data, clinical trials were initiated (Elan) to assess safety and toxicity and to test the efficacy of vaccination with the entire A-beta-42 peptide. Vaccination was performed with pre-aggregated synthetic A-beta-42, injected i.m. (intramuscularly) along with the surface-active saponin QS-21 adjuvant (Hock et al. (2002) Nature Med. 8, 1270-1275; Nicoll et al. (2003) Nature Med. in press) Whereas phase I toxicity trials did not reveal any problems, the subsequent phase II trials were prematurely halted because of serious complications. An inflammatory meningo-encephalitic reaction developed in 16 of 306 vaccinated patients. This adverse reaction was attributed to an auto-immune reaction given the fact that the A-beta-42 peptide moiety is naturally present in the body.

This adverse auto-immune reaction can evidently be avoided by passive immunization, i.e. administration of antibodies directed against A-beta. This approach was shown to be successful in reducing brain A-beta burden in transgenic AD mice (DeMattos et al. (2001) Proc. Natl. Acad. Sci. USA 98, 8850-8855). The underlying mechanisms remain open for speculation since it was thought unlikely that antibodies could cross the blood-brain barrier and target the plaques present in brain. The authors therefore suggested that the antibody created an ‘A-beta sink’ in the plasma which titrated A-beta out of the brain. Subsequently, using gelsolin and GM1, it was demonstrated that any A-beta-binding ligand has the potential to reduce amyloid burden in transgenic AD mice without crossing the blood-brain barrier (Matsuoka et al. (2003) J. Neuroscience 23, 29-33).

Short-term (24 hours) passive immunization appeared to restore cognitive deficits of transgenic AD mice even without affecting the total brain amyloid load (Dodart et al. (2002) Nature Neuroscience 5, 452-457). The result would suggest that smaller, still soluble aggregates of A-beta are targeted first by some antibodies, and also that these are the most toxic forms of A-beta. Hence, clearance of proto-fibrillar A-beta could restore memory, at least in transgenic APP-mice. Concomitant with memory restoration, increased plasma and CSF A-beta levels were observed, supporting the “sink” hypothesis.

Passive rather than active immunization appears to be the most attractive because of the evident absence of auto-immune reaction, the rapid positive effect on memory and the possibility to use any suitable type of antibody with a pre-defined affinity for A-beta. The polypeptides of the present invention are very well suited for this task given their ease of production, high specificity and affinity, high stability combined with low antigenicity and low molecular weight.

Definitive diagnosis of AD still requires post-mortem pathological examination of the brain to demonstrate the presence of amyloid plaques, neurofibrillary tangles, synaptic loss and neuronal degeneration. This is essentially the same procedure as defined by Dr. A. Alzheimer in 1906.

In 1984 the National Institute of Neurological and Communicative Disorders and Stroke and the Alzheimer's Disease and Related Disorders Association (NINCDS-ADRDA) established formal criteria for the diagnosis of AD (reviewed in Petrella et al. (2003) Radiology 226, 315-336). Patients meeting all the following criteria are diagnosed probable AD:
dementia evidenced by examination and testing (e.g. Mini-Mental Test, Blessed Dementia Scale, or similar tests)
impairment of memory and at least one other cognitive function
normal consciousness
onset between 40 and 90 years of age
absence of signs of other diseases that cause dementia (exclusion criterion)

A gradual progressive, cognitive impairment without an identifiable cause will be diagnosed as possible AD. Probable AD is further defined as mild (early), moderate (middle) or severe (late) dementia.

Laboratory analysis is used to objectively define or exclude alternative causes of dementia. ELISA assays of A-beta-42 and phospho-tau in cerebrospinal fluid (CSF), combined with genotyping for ApoE4 (a predisposing genetic factor) appear to be sensitive and specific. The methods are, however, not widely applicable because of the invasive CSF puncture, preventing this to become routine screening.

ELISA for the neural thread protein (AD7C-NTP) (developed by Nymox) demonstrated higher levels in urine from AD patients than from non-AD dementia patients or healthy controls (Munzar et al. (2002) Neurol. Clin. Neurophysiol. 1, 2-8). However, the mean levels were significantly lower in early AD cases, suggesting the test is not reliable for testing for early onset of AD.

No biochemical method is as yet suited for the firm diagnosis of early stages of AD, rather they merely help to confirm the clinical diagnosis of advanced cases. Clearly more advanced techniques are needed to allow early diagnosis before onset of clinical symptoms that signal irreversible brain damage. This is one of the aims of the present invention.

For more information on neurodegenerative diseases and on the role of A-beta therein, reference is inter alia made to Anguiano et al. (2001) Neurobiol Aging 22, 335, Benveniste et al. (1999). Proc. Natl. Acad. Sci. USA 96, 14079-14084, DeMattos et al. (2002) Science 295, 2264-2267, Herms et al. (2002) J. Biol. Chem. 278, 2484-2489, Muruganandam et al (2002) FASEB J. 16, 240-242, Poduslo et al. (2002) J. Neurochem. 81, 61, Small et al. (2001) Alzheimer's disease. Neurobiol Aging 22, 335, Vanhoutte, Dewachter, Borghgraef, Van Leuven, Van der Linden (2003) (Submitted).

It is another aim of the present invention to provide anti-A-beta polypeptides comprising one or more Nanobodies directed towards human A-beta, homologues of said polypeptides, and/or functional portions of said polypeptides, as well as pharmaceutical compositions comprising the same, for diagnosis and therapy of Alzheimer's disease and which overcome the problems of the prior art. Said polypeptides can be used to protect against disorders mediated by A-beta of dysfunction thereof, for example, Alzheimer's disease, by slowing or stopping the disease progression and/or by restoring brain damage, memory and cognition. The polypeptides of the present invention can be used for diagnostic purposes.

It is further an aim to provide methods of production of said anti-A-beta polypeptides, methods and kits for screening and kits for the diagnosis and research of diseases and disorders mediated by A-beta or dysfunction thereof.

Generally, it is an object of the invention to provide pharmacologically active agents, as well as compositions comprising the same, that can be used in the diagnosis, prevention and/or treatment of neurodegenerative diseases such as AD and the further diseases and disorders mentioned herein, and to provide methods for the diagnosis, prevention and/or treatment of such diseases and disorders involving the use and/or administration of such agents and compositions.

In particular, it is an object of the invention to provide such pharmacologically active agents, compositions and/or methods that provide certain advantages compared to the agents, compositions and/or methods currently used and/or known in the art. These advantages will become clear from the further description below.

More in particular, it is an object of the invention to provide therapeutic proteins that can be used as pharmacologically active agents, as well as compositions comprising the same, for the diagnosis, prevention and/or treatment of neurodegenerative diseases such as AD and the further diseases and disorders mentioned herein, and to provide methods for the diagnosis, prevention and/or treatment of such diseases and disorders involving the use and/or administration of such agents and compositions. In the present invention, these therapeutic proteins are (single) domain antibodies and in particular Nanobodies™, and/or are proteins based thereon or comprising the same, as further described below.

In the invention, generally, these objects are achieved by the use of the Nanobodies and polypeptides provided herein.

Thus, it is one object of the present invention to provide Nanobodies against A-beta, in particular against A-beta from a warm-blooded animal, more in particular against A-beta from a mammal, and especially against human A-beta; and to provide proteins and polypeptides comprising or essentially consisting of at least one such Nanobody.

In particular, it is an object of the present invention to provide such Nanobodies and such proteins and/or polypeptides that are suitable for prophylactic, therapeutic and/or diagnostic use in a warm-blooded animal, and in particular in a mammal, and more in particular in a human being.

More in particular, it is an object of the present invention to provide such Nanobodies and such proteins and/or polypeptides that can be used for the prevention, treatment, alleviation and/or diagnosis of one or more diseases, disorders or conditions associated with A-beta and/or mediated by A-beta (such as the diseases, disorders and conditions mentioned herein) in a warm-blooded animal, in particular in a mammal, and more in particular in a human being.

It is also an object of the invention to provide such Nanobodies and such proteins and/or polypeptides that can be used in the preparation of a pharmaceutical or veterinary composition for the prevention and/or treatment of one or more diseases, disorders or conditions associated with and/or mediated by A-beta (such as the diseases, disorders and conditions mentioned herein) in a warm-blooded animal, in particular in a mammal, and more in particular in a human being.

One specific but non-limiting object of the invention is to provide Nanobodies, proteins and/or polypeptides against A-beta that have improved therapeutic and/or pharmacological properties and/or other advantageous properties (such as, for example, improved ease of preparation and/or reduced costs of goods), compared to conventional antibodies against A-beta or fragments thereof, such as Fab′ fragments, F(ab′)₂ fragments, ScFv constructs, “diabodies” and/or other classes of (single) domain antibodies, such as the “dAb's described by Ward et al (supra). These improved and advantageous properties will become clear from the further description herein.

These objects are achieved by the Nanobodies, proteins and polypeptides described herein. These Nanobodies are also referred to herein as “Nanobodies of the invention”; and these proteins and polypeptides are also collectively referred to herein “polypeptides of the invention”.

Thus, in a first aspect, the invention relates to a Nanobody against A-beta, and in particular to a Nanobody against A-beta from a warm-blooded animal, and more in particular to a Nanobody against A-beta from a mammal, and especially to a Nanobody against human A-beta.

In another aspect, the invention relates to a protein or polypeptide that comprises or essentially consists of at least one such Nanobody against A-beta.

It will be clear to the skilled person that for pharmaceutical use, the Nanobodies and polypeptides of the invention are preferably directed against human A-beta; whereas for veterinary purposes, the Nanobodies and polypeptides of the invention are preferably directed against A-beta from the species to be treated.

The efficacy of the Nanobodies and polypeptides of the invention, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person, and for example include the assays and animal models used in the Examples below. It will also be clear to the skilled person that the influence of the Nanobodies and polypeptides of the invention on the formation of amyloid plaques may be determined visually on samples of brain tissue using a microscope, optionally after suitable staining.

Also, according to the invention, Nanobodies and polypeptides that are directed against A-beta from a first species of warm-blooded animal may or may not show cross-reactivity with A-beta from one or more other species of warm-blooded animals. For example, Nanobodies and polypeptides directed against human A-beta may or may not show cross reactivity with A-beta from one or more other species of primates and/or with A-beta from one or more species of animals that are often used in animal models for diseases (for example mouse, rat, rabbit, pig or dog), and in particular in animal models for diseases and disorders associated with A-beta (such as the species and animal models mentioned herein). In this respect, it will be clear to the skilled person that such cross-reactivity, when present, may have advantages from a drug development point of view, since it allows the Nanobodies and polypeptides against human A-beta to be tested in such disease models.

More generally, it is also encompassed within the scope of the invention that Nanobodies and polypeptides directed against A-beta from one species of animal (such as Nanobodies and polypeptides against human A-beta) are used in the treatment of another species of animal, as long as the use of the Nanobodies and/or polypeptides provide the desired effects in the species to be treated.

The present invention is in its broadest sense also not particularly limited to or defined by a specific antigenic determinant, epitope, part, domain, subunit or confirmation (where applicable) of A-beta against which the Nanobodies and polypeptides of the invention are directed. Some of the preferred epitopes and antigenic determinants of A-beta against which the Nanobodies and polypeptides of the present invention may be directed are the epitopes used for immunotherapy, and in particular for passive immunotherapy of AD. For example, as mentioned in the review of Weksler, supra, and in the prior art referred to therein, it is known that there are three major epitopes on A-Beta, i.e. an N-terminal epitope (amino acids 1-6), a central epitope (amino acids 15-25) and a C-terminal region. The Nanobodies of the invention may be directed against either of these epitopes. However, it has been observed that, in the passive immunotherapy of AD with conventional antibodies, antibodies directed against the N-terminal epitope may cause cerebral hemorrhage in APP transgenic mice, whereas conventional antibodies against the C-terminal region have been reported to lack therapeutic effect in APP transgenic mice (see also the references cited in the Weksler review). In this respect, however, it should be noted that generally, due to the differences between Nanobodies and conventional antibodies (as further mentioned herein), Nanobodies may show (increased) efficacy in situations where conventional antibodies do not show efficacy or show insufficient efficacy, and/or Nanobodies may lead to less complications and side-effects than conventional antibodies (for example because of their smaller size and/or because nanobodies and polypeptides comprising Nanobodies can be designed without an Fc-portion and/or an effector function). Therefore, although in selecting the Nanobodies and polypeptide to be used in the present invention, the skilled person should take account of the disadvantages mentioned in the art for conventional antibodies against the N-terminal epitope and the C-terminal region of A-beta, respectively, it is possible and included within the scope of the invention that Nanobodies against the N-terminal epitope and the C-terminal region of A-beta, respectively, do not have the disadvantages described in the art for the corresponding conventional antibodies (or have these disadvantages to a lesser extent), so that they can be used for the purposes mentioned herein.

According to a preferred, but non-limiting embodiment of the invention, the Nanobodies and polypeptides of the invention are directed against the N-terminal epitope of A-beta.

It should also be noted that, as A-beta is formed in vivo by cleavage of APP, the Nanobodies of and polypeptides of the invention may also bind to APP or to specific parts or epitopes thereof. For example, it has been reported in the art that conventional antibodies against the N-terminal epitope or the central region of A-beta also bind to APP (see again the review by Weksler and the references cited therein). Furthermore, although it has been reported that conventional antibodies against the C-terminal region of A-beta are not capable of binding to APP, it should not be excluded that the Nanobodies and polypeptides of the invention against the C-terminal epitope, due to their smaller size and their “cavity binding” properties, are capable of binding to APP as well).

Thus, in its broadest sense, the invention is not limited to any specific mechanism of action or target of the Nanobodies and polypeptides of the invention; in particular, it is included within the scope of the invention that the Nanobodies and polypeptides of the invention provide their desired prophylactic and/or therapeutic action by binding to A-beta, to APP or to both. For example, it is not excluded from the scope of the present invention that the Nanobodies and polypeptides of the invention (also or further) reduce the formation A-beta by reducing the amount and/or the rate of the cleavage of APP.

It is also within the scope of the invention that, where applicable, a Nanobody of the invention can bind to two or more antigenic determinants, epitopes, parts, domains, subunits or confirmations of A-beta. In such a case, the antigenic determinants, epitopes, parts, domains or subunits of A-beta to which the Nanobodies and/or polypeptides of the invention bind may be the essentially same (for example, if A-beta contains repeated structural motifs or is present as a multimer) or may be different (and in the latter case, the Nanobodies and polypeptides of the invention may bind to such different antigenic determinants, epitopes, parts, domains, subunits of A-beta with an affinity and/or specificity which may be the same or different). Also, for example, when A-beta exists in an activated conformation and in an inactive conformation, the Nanobodies and polypeptides of the invention may bind to either one of these confirmation, or may bind to both these confirmations (i.e. with an affinity and/or specificity which may be the same or different). Also, for example, the Nanobodies and polypeptides of the invention may bind to a conformation of A-beta in which it is bound to a pertinent ligand, may bind to a conformation of A-beta in which it not bound to a pertinent ligand, or may bind to both such conformations (again with an affinity and/or specificity which may be the same or different).

It is also expected that the Nanobodies and polypeptides of the invention will generally bind to all naturally occurring or synthetic analogs, variants, mutants, alleles, parts and fragments of A-beta, or at least to those analogs, variants, mutants, alleles, parts and fragments of A-beta that contain one or more antigenic determinants or epitopes that are essentially the same as the antigenic determinant(s) or epitope(s) to which the Nanobodies and polypeptides of the invention bind in A-beta (e.g. in wild-type A-beta). Again, in such a case, the Nanobodies and polypeptides of the invention may bind to such analogs, variants, mutants, alleles, parts and fragments with an affinity and/or specificity that are the same as, or that different from (i.e. higher than or lower than), the affinity and specificity with which the Nanobodies of the invention bind to (wild-type) A-beta. It is also included within the scope of the invention that the Nanobodies and polypeptides of the invention bind to some analogs, variants, mutants, alleles, parts and fragments of A-beta, but not to others.

When A-beta exists in a monomeric form and in one or more multimeric forms, it is within the scope of the invention that the Nanobodies and polypeptides of the invention only bind to A-beta in monomeric form, or that the Nanobodies and polypeptides of the invention in addition also bind to one or more of such multimeric forms. Also, when A-beta can associate with other proteins or polypeptides to form protein complexes, it is within the scope of the invention that the Nanobodies and polypeptides of the invention bind to A-beta in its non-associated state, bind to A-beta in its associated state, or bind to both. In all these cases, the Nanobodies and polypeptides of the invention may bind to such multimers or associated protein complexes with an affinity and/or specificity that may be the same as or different from (i.e. higher than or lower than) the affinity and/or specificity with which the Nanobodies and polypeptides of the invention bind to A-beta in its monomeric and non-associated state.

Generally, the Nanobodies and polypeptides of the invention will at least bind to those forms (including monomeric, multimeric and associated forms) that are the most relevant from a biological and/or therapeutic point of view, as will be clear to the skilled person.

It is also within the scope of the invention to use parts, fragments, analogs, mutants, variants, alleles and/or derivatives of the Nanobodies and polypeptides of the invention, and/or to use proteins or polypeptides comprising or essentially consisting of the same, as long as these are suitable for the uses envisaged herein. Such parts, fragments, analogs, mutants, variants, alleles, derivatives, proteins and/or polypeptides will be described in the further description herein.

As discussed in more detail herein, the Nanobodies of the invention generally comprise a single amino acid chain, that can be considered to comprise “framework sequences” or “FR” (which are generally as described herein) and “complementarity determining regions” of CDR's. Some preferred CDR's present in the Nanobodies of the invention are as described herein. More generally, and with reference to the further definitions given herein, the CDR sequences present in the Nanobodies of the invention are obtainable/can be obtained by a method comprising the steps of:
a) providing at least one V_{HH} domain directed against A-beta, by a method generally comprising the steps of (i) immunizing a mammal belonging to the Camelidae with A-beta or a part or fragment thereof, so as to raise an immune response and/or antibodies (and in particular heavy chain antibodies) against A-beta; (ii) obtaining a biological sample from the mammal thus immunized, wherein said sample comprises heavy chain antibody sequences and/or V_{HH} sequences that are directed against A-beta; and (iii) obtaining (e.g isolating) heavy chain antibody sequences and/or V_{HH} sequences that are directed against A-beta from said biological sample; and/or by a method generally comprising the steps of (i) screening a library comprising heavy chain antibody sequences and/or V_{HH} sequences for heavy chain antibody sequences and/or V_{HH} sequences that are directed against A-beta or against at least one part or fragment thereof; and (ii) obtaining (e.g. isolating) heavy chain antibody sequences and/or V_{HH} sequences that are directed against A-beta from said library;
b) optionally subjecting the heavy chain antibody sequences and/or V_{HH} sequences against A-beta thus obtained to affinity maturation, to mutagenesis (e.g. random mutagenesis or site-directed mutagenesis) and/or any other technique(s) for increasing the affinity and/or specificity of the heavy chain antibody sequences and/or V_{HH} sequences for A-beta;
c) determining the sequences of the CDR's of the heavy chain antibody sequences and/or V_{HH} sequences against A-beta thus obtained; and optionally
d) providing a Nanobody in which at least one, preferably at least two, and more preferably all three of the CDR's (i.e. CDR1, CDR2 and CDR3, and in particular at least CDR3) has a sequence that has been determined in step c).

Usually, in step d), all CDR sequences present in a Nanobody of the invention will be derived from the same heavy chain antibody or V_{HH} sequence. However, the invention in its broadest sense is not limited thereto. It is for example also possible (although often less preferred) to suitably combine, in a Nanobody of the invention, CDR's from two or three different heavy chain antibodies or V_{HH} sequences against A-beta and/or to suitably combine, in a Nanobody of the invention, one or more CDR's derived from heavy chain antibodies or V_{HH} sequences (an in particular at least CDR3) with one or more CDR's derived from a different source (for example synthetic CDR's or CDR's derived from a human antibody or V_{H} domain).

According to a non-limiting but preferred embodiment of the invention, the CDR sequences in the Nanobodies of the invention are such that the Nanobody of the invention binds to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the Nanobody of the invention against A-beta can be determined in a manner known per se, for example using the assay described herein.

In a preferred but non-limiting aspect, the invention relates to a Nanobody (as defined herein) against A-beta, which consist of 4 framework regions (FR1 to FR4 respectively) and 3 complementarity determining regions (CDR1 to CDR3 respectively), in which:
i) CDR1 is an amino acid sequence chosen from the group consisting of:

| | |
|---|---|
| GGTFSSVGMG | [SEQ ID NO:37] |
| GFTFSNYGMI | [SEQ ID NO:38] |
| GGTFSSIGMG | [SEQ ID NO:39] |
| GFTFSNYWMY | [SEQ ID NO:40] |
| GFTLSSITMT | [SEQ ID NO:41] |
| GRTFSIYNMG | [SEQ ID NO:42] |
| GRTFTSYNMG | [SEQ ID NO:43] |
| GFTFSNYWMY | [SEQ ID NO:44] |
| GGTFSSIGMG | [SEQ ID NO:45] |
| GGIYRVNTVN | [SEQ ID NO:46] |
| GFTFSNYWMY | [SEQ ID NO:47] |
| GFTLSSITMT | [SEQ ID NO:48] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
ii) CDR2 is an amino acid sequence chosen from the group consisting of:

| | |
|---|---|
| AISRSGDSTYYAGSVKG | [SEQ ID NO:49] |
| GISDGGRSTSYADSVKG | [SEQ ID NO:50] |
| AISRSGDSTYYADSVKG | [SEQ ID NO:51] |
| TISPRAAVTYYADSVKG | [SEQ ID NO:52] |
| TINSGGDSTTYADSVKG | [SEQ ID NO:53] |
| TITRSGGSTYYADSVKG | [SEQ ID NO:54] |
| TISRSGGSTYYADSVKG | [SEQ ID NO:55] |
| TISPRAGSTYYADSVKG | [SEQ ID NO:56] |
| AISRSGDSTYYADSVKG | [SEQ ID NO:57] |
| TITRAGSTNYVESVKG | [SEQ ID NO:58] |
| TISPRAANTYYADSVKG | [SEQ ID NO:59] |
| TINSGGDSTTYADSVKG | [SEQ ID NO:60] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
iii) CDR3 is an amino acid sequence chosen from the group consisting of:

| | |
|---|---|
| RPAGTPINIRRAYNY | [SEQ ID NO:61] |
| AYGRGTYDY | [SEQ ID NO:62] |
| RPAGTAINIRRSYNY | [SEQ ID NO:63] |
| SLKYWHRPQSSDFAS | [SEQ ID NO:64] |
| GTYYSRAYYR | [SEQ ID NO:65] |
| ARIGAAVNIPSEYDS | [SEQ ID NO:66] |
| RPAGTPINIRRAYNY | [SEQ ID NO:67] |
| SLIYKARPQSSDFVS | [SEQ ID NO:68] |
| RPAGTAINIRRSYNY | [SEQ ID NO:69] |
| NGRWRSWSSQRDY | [SEQ ID NO:70] |
| SLRYRDRPQSSDFLF | [SEQ ID NO:71] |
| GTYYSRAYYR | [SEQ ID NO:72] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s).

Thus, some particularly preferred, but non-limiting CDR sequences and combinations of CDR sequences that are present in the Nanobodies of the invention are as listed in Table A-1 below.

TABLE A-1
preferred CDR sequences and combinations of CDR sequence

| | | |
|---|---|---|
| CDR1 | CDR2 | CDR3 |

| Clone | | SEQ ID | | SEQ ID | | SEQ ID |
|---|---|---|---|---|---|---|
| designation | Sequence | NO | Sequence | NO | Sequence | NO |
| MP1 Aβ D7 | GGTFSSVGMG | 37 | AISRSGDSTYYAGSVKG | 49 | RPAGTPINIRRAYNY | 61 |
| MP1 Aβ C2 | GFTFSNYGMI | 38 | GISDGGRSTSYADSVKG | 50 | AYGRGTYDY | 62 |
| MP1 Aβ H3 | GGTFSSIGMG | 39 | AISRSGDSTYYADSVKG | 51 | RPAGTAINIRRSYNY | 63 |
| MP1 Aβ H6 | GFTFSNYWMY | 40 | TISPRAAVTYYADSVKG | 52 | SLKYWHRPQSSDFAS | 64 |
| MP1 Aβ B12 | GFTLSSITMT | 41 | TINSGGDSTTYADSVKG | 53 | GTYYSRAYYR | 65 |
| MP2 Aβ C2 | GRTFSIYNMG | 42 | TITRSGGSTYYADSVKG | 54 | ARIGAAVNIPSEYDS | 66 |
| MP4 Aβ F12 | GRTFTSYNMG | 43 | TISRSGGSTYYADSVKG | 55 | RPAGTPINIRRAYNY | 67 |
| BA PMP2 C7 | GFTFSNYWMY | 44 | TISPRAGSTYYADSVKG | 56 | SLIYKARPQSSDFVS | 68 |
| BA PMP2 D2 | GGTFSSIGMG | 45 | AISRSGDSTYYADSVKG | 57 | RPAGTAINIRRSYNY | 69 |
| BA PMP2 E10 | GGIYRVNTVN | 46 | TITRAGSTNYVESVKG | 58 | NGRWRSWSSQRDY | 70 |
| BA PMP2 G6 | GFTFSNYWMY | 47 | TISPRAANTYYADSVKG | 59 | SLRYRDRPQSSDFLF | 71 |
| BA PMP2 D6 | GFTLSSITMT | 48 | TINSGGDSTTYADSVKG | 60 | GTYYSRAYYR | 72 |

Thus, in the Nanobodies of the invention, at least one of the CDR1, CDR2 and CDR3 sequences present is chosen from the group consisting of the CDR1, CDR2 and CDR3 sequences, respectively, listed in Table A-1; or from the group of CDR1, CDR2 and CDR3 sequences, respectively, that have at least 80%, preferably at least 90%, more preferably at least 95%, even more preferably at least 99% “sequence identity” (as defined herein) with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” (as defined herein) with at least one of the CDR1, CDR2 and CDR3 sequences, respectively, listed in Table A-1.

In particular, in the Nanobodies of the invention, at least the CDR3 sequence present is chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1; and/or from the group consisting of the CDR3 sequences that have 3, 2 or only 1 amino acid difference(s) with at least one of the CDR3 sequences listed in Table A-1.

Preferably, in the Nanobodies of the invention, at least two of the CDR1, CDR2 and CDR3 sequences present are chosen from the group consisting of the CDR1, CDR2 and CDR3 sequences, respectively, listed in Table A-1 or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” with at least one of the CDR1, CDR2 and CDR3 sequences, respectively, listed in Table A-1.

In particular, in the Nanobodies of the invention, at least the CDR3 sequence present is chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1, respectively; and at least one of the CDR1 and CDR2 sequences present is chosen from the group consisting of the CDR1 and CDR2 sequences, respectively, listed in Table A-1 or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Most preferably, in the Nanobodies of the invention, all three CDR1, CDR2 and CDR3 sequences present are chosen from the group consisting of the CDR1, CDR2 and CDR3 sequences, respectively, listed in Table A-1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-1.

Even more preferably, in the Nanobodies of the invention, at least one of the CDR1, CDR2 and CDR3 sequences present is chosen from the group consisting of the CDR1, CDR2 and CDR3 sequences, respectively, listed in Table A-1. Preferably, in this embodiment, at least one or preferably both of the other two CDR sequences present are chosen from CDR sequences that that have at least 80%, preferably at least 90%, more preferably at least 95%, even more preferably at least 99% sequence identity with at least one of the corresponding CDR sequences, respectively, listed in Table A-1; and/or from the group consisting of the CDR sequences that have 3, 2 or only 1 amino acid difference(s) with at least one of the corresponding sequences, respectively, listed in Table A-1.

In particular, in the Nanobodies of the invention, at least the CDR3 sequence present is chosen from the group consisting of the CDR3 listed in Table A-1. Preferably, in this embodiment, at least one and preferably both of the CDR1 and CDR2 sequences present are chosen from the groups of CDR1 and CDR2 sequences, respectively, that that have at least 80%, preferably at least 90%, more preferably at least 95%, even more preferably at least 99% sequence identity with the CDR1 and CDR2 sequences, respectively, listed in listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Even more preferably, in the Nanobodies of the invention, at least two of the CDR1, CDR2 and CDR3 sequences present are chosen from the group consisting of the CDR1, CDR2 and CDR3 sequences, respectively, listed in Table A-1. Preferably, in this embodiment, the remaining CDR sequence present are chosen from the group of CDR sequences that that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with at least one of the corresponding sequences listed in Table A-1.

In particular, in the Nanobodies of the invention, at least the CDR3 sequence is chosen from the group consisting of the CDR3 sequences listed in Table A-1, and either the CDR1 sequence or the CDR2 sequence is chosen from the group consisting of the CDR1 and CDR2 sequences, respectively, listed in Table A-1. Preferably, in this embodiment, the remaining CDR sequence present are chosen from the group of CDR sequences that that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with the corresponding CDR sequences listed in Table A-1.

Even more preferably, in the Nanobodies of the invention, all three CDR1, CDR2 and CDR3 sequences present are chosen from the group consisting of the CDR1, CDR2 and CDR3 sequences, respectively, listed in Table A-1.

Also, generally, the combinations of CDR's listed in Table A-1 (i.e. those mentioned on the same line in Table A-1) are preferred. Thus, it is generally preferred that, when a CDR in a Nanobody of the invention is a CDR sequence mentioned in Table A-1 or is chosen from the group of CDR sequences that have at least 80%, preferably at least 90%, more preferably at least 95%, even more preferably at least 99% sequence identity with a CDR sequence listed in Table A-1; and/or from the group consisting of CDR sequences that have 3, 2 or only 1 amino acid difference(s) with a CDR sequence listed in Table A-1, that at least one and preferably both of the other CDR's are chosen from the CDR sequences that belong to the same combination in Table A-1 (i.e. mentioned on the same line in Table A-1) or are chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR's mentioned in Table A-1.

Thus, by means of non-limiting examples, a Nanobody of the invention can for example comprise a CDR1 sequence that has more than 80% sequence identity with one of the CDR1 sequences mentioned in Table A-1, a CDR2 sequence that has 3, 2 or 1 amino acid difference with one of the CDR2 sequences mentioned in Table A-1 (but belonging to a different combination), and a CDR3 sequence.

Some preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with one of the CDR2 sequences mentioned in Table A-1 (but belonging to a different combination); and a CDR3 sequence that has more than 80% sequence identity with one of the CDR3 sequences mentioned in Table A-1 (but belonging to a different combination); or (2) a CDR1 sequence that has more than 80% sequence identity with one of the CDR1 sequences mentioned in Table A-1; a CDR2 sequence, and one of the CDR3 sequences listed in Table A-1; or (3) a CDR1 sequence; a CDR2 sequence that has more than 80% sequence identity with one of the CDR2 sequence listed in Table A-1; and a CDR3 sequence that has 3, 2 or 1 amino acid differences with the CDR3 sequence mentioned in Table A-1 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with the CDR2 sequence mentioned in Table A-1 that belongs to the same combination; and a CDR3 sequence that has more than 80% sequence identity with the CDR3 sequence mentioned in Table A-1 that belongs to the same combination; (2) a CDR1 sequence; a CDR 2 listed in Table A-1 and a CDR3 sequence listed in Table A-1 (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-1; the CDR2 sequence listed in Table A-1 that belongs to the same combination; and a CDR3 sequence mentioned in Table A-1 that belongs to a different combination; or (2) a CDR1 sequence mentioned in Table A-1; a CDR2 sequence that has 3, 2 or 1 amino acid differences with the CDR2 sequence mentioned in Table A-1 that belongs to the same combination; and more than 80% sequence identity with the CDR3 sequence listed in Table A-1 that belongs to same different combination.

Particularly preferred Nanobodies of the invention may for example comprise a CDR1 sequence mentioned in Table A-1, a CDR2 sequence that has more than 80% sequence identity with the CDR2 sequence mentioned in Table A-1 that belongs to the same combination; and the CDR3 sequence mentioned in Table A-1 that belongs to the same.

In the most preferred in the Nanobodies of the invention, the CDR1, CDR2 and CDR3 sequences present are chosen from the one of the combinations of CDR1, CDR2 and CDR3 sequences, respectively, listed in Table A-1.

Preferably, when a CDR sequence is chosen from the group of CDR sequences that have at least 80%, preferably at least 90%, more preferably at least 95%, even more preferably at least 99% sequence identity (as defined herein) with one of the CDR sequences listed in Table A-1; and/or when a CDR sequence is chosen from the group consisting of CDR sequences that have 3, 2 or only 1 amino acid difference(s) with one of the CDR sequences listed in Table A-1:
i) any amino acid substitution is preferably a conservative amino acid substitution (as defined herein); and/or
ii) said amino acid sequence preferably only contains amino acid substitutions, and no amino acid deletions or insertions, compared to the CDR sequence listed in Table A-1.

According to a non-limiting but preferred embodiment of the invention, the CDR sequences in the Nanobodies of the invention are as defined above and are also such that the Nanobody of the invention binds to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the Nanobody of the invention against A-beta can be determined in a manner known per se, for example using the assay described herein.

According to another preferred, but non-limiting embodiment of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 and 17 amino acid residues; and/or (c) CDR3 has a length of between 2 and 35 amino acid residues, and usually between 3 and 30 amino acid residues, such as between 6 and 23 amino acid residues.

Nanobodies with the above CDR sequences preferably have framework sequences that are as further defined herein.

In another aspect, the invention relates to a Nanobody with an amino acid sequence that is chosen from the group consisting of SEQ ID NO's: 73 to 105 or from the group consisting of from amino acid sequences that have more than 80%, preferably more than 90%, more preferably more than 95%, such as 99% or more sequence identity (as defined herein) with one or more of the amino acid sequences of SEQ ID NO's: 73 to 105.

According to a specific, but non-limiting embodiment, the latter amino acid sequences have been “humanized”, as further described herein. Some preferred, but non-limiting examples of such humanized Nanobodies are given in SEQ ID NO's: 85 to 105.

In the invention, the Nanobodies of SEQ ID NO's: 80 to 84 and humanized variants thereof are particularly preferred.

The polypeptides of the invention comprise or essentially consist of at least one Nanobody of the invention.

Generally, proteins or polypeptides that comprise or essentially consist of a single Nanobody (such as a single Nanobody of the invention) will be referred to herein as “monovalent” proteins or polypeptides or as “monovalent constructs”. Proteins and polypeptides that comprise or essentially consist of two or more Nanobodies (such as at least two Nanobodies of the invention or at least one Nanobody of the Invention and at least one other Nanobody) will be referred to herein as “multivalent” proteins or polypeptides or as “multivalent constructs”, and these may provide certain advantages compared to the corresponding monovalent Nanobodies of the invention. Some non-limiting examples of such multivalent constructs will become clear from the further description herein.

According to another specific, but non-limiting embodiment, a polypeptide of the invention comprises or essentially consists of at least one Nanobody of the invention and at least one other Nanobody (i.e. directed against another epitope, antigen, target, protein or polypeptide). Such proteins or polypeptides are also referred to herein as “multispecific” proteins or polypeptides or as “multispecific constructs”, and these may provide certain advantages compared to the corresponding monovalent Nanobodies of the invention. Again, some non-limiting examples of such multispecific constructs will become clear from the further description herein.

According to yet another specific, but non-limiting embodiment, a polypeptide of the invention comprises or essentially consists of at least one Nanobody of the invention, optionally one or more further Nanobodies, and at least one other amino acid sequence (such as a protein or polypeptide) that confers at least one desired property to the Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

It is also possible to combine two or more of the above embodiments, for example to provide a trivalent bispecific construct comprising two Nanobodies of the invention and one other Nanobody, and optionally one or more other amino acid sequences. Further non-limiting examples of such constructs, as well as some constructs that are particularly preferred within the context of the present invention, will become clear from the further description herein.

In the above constructs, the one or more Nanobodies and/or other amino acid sequences may be directly linked or linked via one or more linker sequences. Some suitable but non-limiting examples of such linkers will become clear from the further description herein.

In one preferred embodiment of the invention, a polypeptide of the invention comprises one or more (such as two or preferably one) Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 189) and FC5 (SEQ ID NO: 190) are some preferred non-limiting examples.

In another preferred embodiment of the invention, a polypeptide of the invention comprises one or more (such as two or preferably one) Nanobodies of the invention linked (optionally via one or more suitable linker sequences) to one or more (such as two and preferably one) amino acid sequences that confer an increased half-life in vivo to the resulting polypeptide of the invention. In particular, said amino acid sequences that confer an increased half-life in vivo to the resulting polypeptide of the invention may be one or more (such as two and preferably one) Nanobodies, and in particular Nanobodies directed against a human serum protein such as human serum albumin, of which SEQ ID NO's 110 to 116 are some non-limiting examples, and PMP6A6 (“ALB-1”, SEQ ID NO: 34), ALB-8 (a humanized version of A1B-1, SEQ ID NO:35) and PMP6A8 (“ALB-2”, SEQ ID NO:36) are some preferred non-limiting examples

In yet another preferred embodiment of the invention, a polypeptide of the invention comprises one or more (such as two or preferably one) Nanobodies of the invention, one or more (such as two and preferably one) amino acid sequences that allow the resulting polypeptide of the invention to cross the blood brain barrier, and one or more (such as two and preferably one) amino acid sequences that confer an increased half-life in vivo to the resulting polypeptide of the invention (optionally linked via one or more suitable linker sequences). Again, said one or more amino acid sequences that allow the resulting polypeptides of the invention to cross the blood brain barrier may be one or more (such as two and preferably one) Nanobodies (as mentioned herein), and said amino acid sequences that confer an increased half-life in vivo to the resulting polypeptide of the invention may be one or more (such as two and preferably one) Nanobodies (also as mentioned herein).

According to a non-limiting but preferred embodiment of the invention, the polypeptides of the invention are preferably such that they bind to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹) preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 μM. The affinity of the polypeptide of the invention against A-beta can be determined in a manner known per se, for example using the assay described herein.

Some preferred, but non-limiting examples of polypeptides of the invention are the polypeptides of SEQ ID NO's: 117 to 183, in which:
SEQ ID NO's: 150 to 165 are some examples of multivalent (and in particular bivalent) polypeptides of the invention;
SEQ ID NO's: 117 to 149 and SEQ ID NO's: 166 to 173 are some examples of bispecific polypeptides of the invention, comprising one or two Nanobodies of the invention and a Nanobody directed against (human or mouse, respectively) serum albumin;
SEQ ID NO's: 174 to 177 are some examples of bispecific polypeptides of the invention, comprising one or two Nanobodies of the invention and a Nanobody that allows the polypeptide of the invention to cross the blood brain barrier; and
SEQ ID NO's: 178 to 183 are some examples of trispecific polypeptides of the invention, comprising one or two Nanobodies of the invention, a Nanobody directed against human serum albumin, and a Nanobody that allows the polypeptide of the invention to cross the blood brain barrier.

Other polypeptides of the invention may for example be chosen from the group consisting of amino acid sequences that have more than 80%, preferably more than 90%, more preferably more than 95%, such as 99% or more “sequence identity” (as defined herein) with one or more of the amino acid sequences of SEQ ID NO's: 117 to 183, in which the Nanobodies comprised within said amino acid sequences are preferably as defined herein.

In another aspect, the invention relates to a nucleic acid that encodes a Nanobody of the invention and/or a polypeptide of the invention. Such a nucleic acid will also be referred to herein as a “nucleic acid of the invention” and may for example be in the form of a genetic construct, as defined herein.

In another aspect, the invention relates to host or host cell that expresses or that is capable of expressing a Nanobody of the invention and/or a polypeptide of the invention; and/or that contains a nucleic acid of the invention. Some preferred but non-limiting examples of such hosts or host cells will become clear from the further description herein.

The invention further relates to a product or composition containing or comprising at least one Nanobody of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention further relates to methods for preparing or generating the Nanobodies, polypeptides, nucleic acids, host cells, products and compositions described herein. Some preferred but non-limiting examples of such methods will become clear from the further description herein.

The invention further relates to applications and uses of the Nanobodies, polypeptides, nucleic acids, host cells, products and compositions described herein, as well as to methods for the prevention and/or treatment for diseases and disorders associated with A-beta. Some preferred but non-limiting applications and uses will become clear from the further description herein.

Other aspects, embodiments, advantages and applications of the invention will also become clear from the further description hereinbelow.

### DETAILED DESCRIPTION OF THE INVENTION

The above and other aspects, embodiments and advantages of the invention will become clear from the further description hereinbelow, in which:
a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd.Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005), as well as to the general background art cited herein;
b) Unless indicated otherwise, the term “immunoglobulin sequence”—whether it used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation;
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein;
d) Amino acid residues will be indicated according to the standard three-letter or one-letter amino acid code, as mentioned in Table A-2;

TABLE A-2
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residu can generally be considered essentially uncharged at a pH of about 6.5.

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).

Alternatively, the degree of sequence identity between two or more nucleotide sequences may be calculated using a known computer algorithm for sequence alignment such as NCBI Blast v2.0, using standard settings.

Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
f) For the purposes of comparing two or more amino acid sequences, the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of nucleotides in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.

Alternatively, the degree of sequence identity between two amino acid sequences may be calculated using a known computer algorithm, such as those mentioned above for determining the degree of sequence identity for nucleotide sequences, again using standard settings.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: H is, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into H is; Asp into Glu; Cys into Ser; Gln into Asn; Glu into Asp; Gly into Ala or into Pro; H is into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

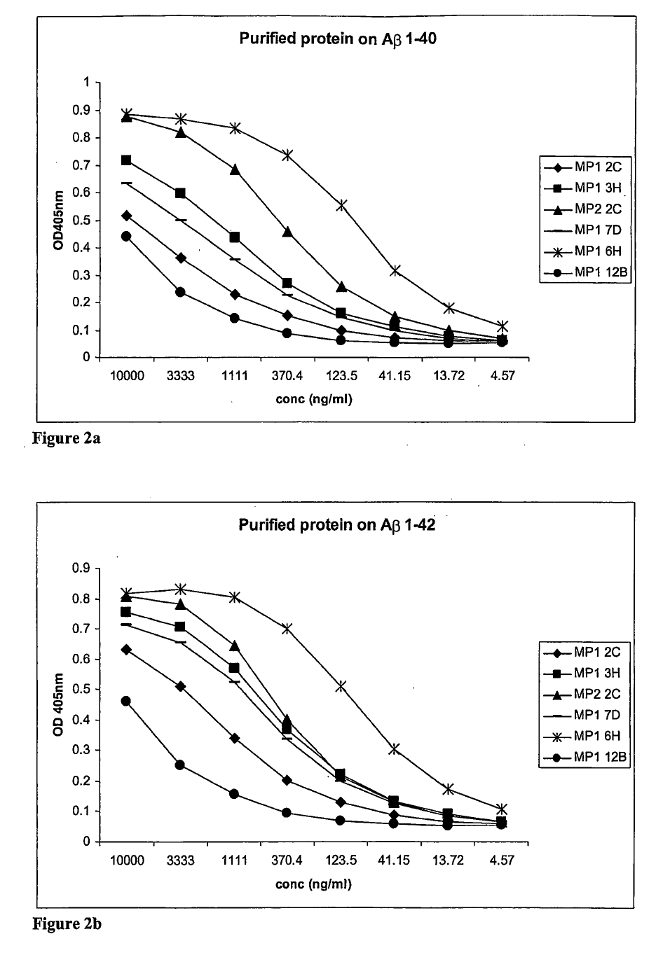

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J. Molec. Biol. 157: 105-132, 1981, and Goldman et al., Ann. Rev. Biophys. Chem. 15: 321-353, 1986, all incorporated herein in their entirety by reference. Information on the primary, secondary and tertiary structure of Nanobodies given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions;
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gelelectrophoresis;
j) The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds.
k) The term ‘antigenic determinant’ refers to the epitope on the antigen recognized by the antigen-binding molecule (such as a Nanobody or a polypeptide of the invention) and more in particular by the antigen-binding site of said molecule. The terms “antigenic determinant” and “epitope” may also be used interchangeably herein.
l) An amino acid sequence (such as a Nanobody, an antibody, a polypeptide of the invention, or generally an antigen binding protein or polypeptide or a fragment thereof) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
m) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the Nanobodies and/or polypeptides of the invention) will bind with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ liters/mol is generally considered to indicate non-specific binding. Preferably, a Nanobody or polypeptide of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.
n) As further described herein, the amino acid sequence and structure of a Nanobody can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively;
o) As also further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
p) The amino acid residues of a Nanobody are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to above (see for example FIG. 2 of said reference). According to this numbering, FR1 of a Nanobody comprises the amino acid residues at positions 1-30, CDR1 of a Nanobody comprises the amino acid residues at positions 31-36, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].

Alternative methods for numbering the amino acid residues of V_{H} domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise; and
q) The Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter alia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and applicant; WO 01/90190 by the National Research Council of Canada; WO 03/025020 (=EP 1 433 793) by the Institute of Antibodies; as well as WO 04/041867, WO 04/041862, WO04/041865, WO 04/041863, WO 04/062551 by applicant and the further published patent applications by applicant; Hamers-Casterman et al., Nature 1993 Jun. 3; 363 (6428): 446-8; Davies and Riechmami, FEBS Lett. 1994 Feb. 21; 339(3): 285-90; Muyldermans et al., Protein Eng. 1994 September; 7(9): 1129-3; Davies and Riechmann, Biotechnology (NY) 1995 May; 13(5): 475-9; Gharoudi et al., 9th Forum of Applied Biotechnology, Med. Fac. Landbouw Univ. Gent. 1995; 60/4a part I: 2097-2100; Davies and Riechmann, Protein Eng. 1996 June; 9(6): 531-7; Desmyter et al., Nat Struct Biol. 1996 September; 3(9): 803-11; Sheriff et al., Nat Struct Biol. 1996 September; 3(9): 733-6; Spinelli et al., Nat Struct Biol. 1996 September; 3(9): 752-7; Arbabi Ghahroudi et al., FEBS Lett. 1997 Sep. 15; 414(3): 521-6; Vu et al., Mol Immol. 1997 November-December; 34(16-17): 1121-31; Atarhouch et al., Journal of Camel Practice and Research 1997; 4: 177-182; Nguyen et al., J. Mol. Biol. 1998 Jan. 23; 275(3): 413-8; Lauwereys et al., EMBO J. 1998 Jul. 1; 17(13): 3512-20; Frenken et al., Res Immunol. 1998 July-August; 149(6):589-99; Transue et al., Proteins 1998 Sep. 1; 32(4): 515-22; Muyldernans and Lauwereys, J. Mol. Recognit. 1999 March-April; 12 (2): 131-40; van der Linden et al., Biochim. Biophys. Acta 1999 Apr. 12; 1431(1): 37-46; Decaimiere et al., Structure Fold. Des. 1999 Apr. 15; 7(4): 361-70; Ngyuen et al., Mol. Immunol. 1999 June; 36(8): 515-24; Woolven et al., Immunogenetics 1999 October; 50 (1-2): 98-101; Riechmann and Muyldermans, J. Immunol. Methods 1999 Dec. 10; 231 (1-2): 25-38; Spinelli et al., Biochemistry 2000 Feb. 15; 39(6): 1217-22; Frenken et al., J. Biotechnol. 2000 Feb. 28; 78(1): 11-21; Nguyen et al., EMBO J. 2000 Mar. 1; 19(5): 921-30; van der Linden et al., J. Immunol. Methods 2000 Jun. 23; 240 (1-2): 185-95; Decanniere et al., J. Mol. Biol. 2000 Jun. 30; 300 (1): 83-91; van der Linden et al., J. Biotechnol. 2000 Jul. 14; 80(3): 261-70; Harmsen et al., Mol. ImmLmol. 2000 August; 37(10): 579-90; Perez et al., Biochemistry 2001 Jan. 9; 40(1): 74-83; Conrath et al., J. Biol. Chem. 2001 Mar. 9; 276 (10): 7346-50; Muyldermans et al., Trends Biochem Sci. 2001 April; 26(4):230-5; Muyldermans S., J. Biotechnol. 2001 June; 74 (4): 277-302; Desmyter et al., J. Biol. Chem. 2001 Jul. 13; 276 (28): 26285-90; Spinelli et al., J. Mol. Biol. 2001 Aug. 3; 311 (1): 123-9; Conrath et al., Antimicrob Agents Chemother. 2001 October; 45 (10): 2807-12; Decanniere et al., J. Mol. Biol. 2001 Oct. 26; 313(3): 473-8; Nguyen et al., Adv Immunol. 2001; 79: 261-96; Muruganandam et al., FASEB J. 2002 February; 16 (2): 240-2; Ewert et al., Biochemistry 2002 Mar. 19; 41 (11): 3628-36; Dumoulin et al., Protein Sci. 2002 March; 11 (3): 500-15; Cortez-Retamozo et al., Int. J. Cancer. 2002 Mar. 20; 98 (3): 456-62; Su et al., Mol. Biol. Evol. 2002 March; 19 (3): 205-15; van der Vaalt J M., Methods Mol. Biol. 2002; 178: 359-66; Vranken et al., Biochemistry 2002 Jul. 9; 41 (27): 8570-9; Nguyen et al., Immunogenetics 2002 April; 54 (1): 39-47; Renisio et al., Proteins 2002 Jun. 1; 47 (4): 546-55; Desmyter et al., J. Biol. Chem. 2002 Jun. 28; 277 (26): 23645-50; Ledeboer et al., J. Dairy Sci. 2002 June; 85 (6): 1376-82; De Genst et al., J. Biol. Chem. 2002 Aug. 16; 277 (33): 29897-907; Ferrat et al., Biochem. J. 2002 Sep. 1; 366 (Pt 2): 415-22; Thomassen et al., Enzyme and Microbial Technol. 2002; 30: 273-8; Harnsen et al., Appl. Microbiol. Biotechnol. 2002 December; 60 (4): 449-54; Jobling et al., Nat. Biotechnol. 2003 January; 21 (1): 77-80; Conrath et al., Dev. Comp. Immunol. 2003 February; 27 (2): 87-103; Pleschberger et al., Bioconjug. Chem. 2003 March-April; 14 (2): 440-8; Lah et al., J. Biol. Chem. 2003 Apr. 18; 278 (16): 14101-11; Nguyen et al., Immunology. 2003 May; 109 (1): 93-101; Joosten et al., Microb. Cell Fact. 2003 Jan. 30; 2 (1): 1; Li et al., Proteins 2003 Jul. 1; 52 (1): 47-50; Loris et al., Biol. Chem. 2003 Jul. 25; 278 (30): 28252-7; van Koningsbniggen et al., J. Immunol. Methods. 2003 August; 279 (1-2): 149-61; Dunoulin et al., Nature. 2003 Aug. 14; 424 (6950): 783-8; Bond et al., J. Mol. Biol. 2003 Sep. 19; 332 (3): 643-55; Yau et al., J. Immunol. Methods. 2003 Oct. 1; 281 (1-2): 161-75; Dekker et al., J. Virol. 2003 November; 77 (22): 12132-9; Meddeb-Moue111 et al., Toxicon. 2003 December; 42 (7): 785-91; Verheesen et al., Biochlm. Biophys. Acta 2003 Dec. 5; 1624 (1-3): 21-8; Zhang et al., J Mol. Biol. 2004 Jan. 2; 335 (1): 49-56; Stijlemans et al., J Biol. Chem. 2004 Jan. 9; 279 (2): 1256-61; Cortez-Retamozo et al., Cancer Res. 2004 Apr. 15; 64 (8): 2853-7; Spinelli et al., FEBS Lett. 2004 Apr. 23; 564 (1-2): 35-40; Pleschberger et al., Bioconjug. Chem. 2004 May-June; 15 (3): 664-71; Nicaise et al., Protein Sci. 2004 July; 13 (7): 1882-91; Omidfar et al., TLunour Biol. 2004 July-August; 25 (4): 179-87; Omidfar et al., Tumour Biol. 2004 September-December; 25(5-6): 296-305; Szynol et al., Antimicrob Agents Chemother. 2004 September; 48(9):3390-5; Saerens et al., J. Biol. Chem. 2004 Dec. 10; 279 (50): 51965-72; De Genst et al., J. Biol. Chem. 2004 Dec. 17; 279 (51): 53593-601; Dolls et al., Appl. Environ. Microbiol. 2005 January; 71(1): 442-50; Joosten et al., Appl Microbiol Biotechnol. 2005 January; 66(4): 384-92; Dumoulin et al., J. Mol. Biol. 2005 Feb. 25; 346 (3): 773-88; Yau et al., J Immunol Methods. 2005 February; 297 (1-2): 213-24; De Genst et al., J. Biol. Chem. 2005 Apr. 8; 280 (14): 14114-21; Huang et al., Eur. J. Hun. Genet. 2005 Apr. 13; Dolk et al., Proteins. 2005 May 15; 59 (3): 555-64; Bond et al., J. Mol. Biol. 2005 May 6; 348(3):699-709; Zarebski et al., J. Mol. Biol. 2005 Apr. 21; [E-publication ahead of print].

In accordance with the terminology used in the above references, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFv's or conventional antibody fragments (such as Fab- or F(ab′)₂-fragments):
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
V_{HH} domains and Nanobodies can be expressed from a single gene and require no post-translational folding or modifications;
V_{HH} domains and Nanobodies can easily be engineered into multivalent and multispecific formats (as further discussed herein);
V_{HH} domains and Nanobodies are highly soluble and do not have a tendency to aggregate (as with the mouse-derived antigen-binding domains” described by Ward et al., Nature, Vol. 341, 1989, p. 544);
V_{HH} domains and Nanobodies are highly stable to heat, pH, proteases and other denaturing agents or conditions (see for example Ewert et al, supra);
V_{HH} domains and Nanobodies are easy and relatively cheap to prepare, even on a scale required for production. For example, V_{HH} domains, Nanobodies and proteins/polypeptides containing the same can be produced using microbial fermentation (e.g. as further described below) and do not require the use of mammalian expression systems, as with for example conventional antibody fragments;
V_{HH} domains and Nanobodies are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof;
V_{HH} domains and Nanobodies can show so-called cavity-binding properties (inter alia due to their extended CDR3 loop, compared to conventional V_{H} domains) and can therefore also access targets and epitopes not accessable to conventional 4-chain antibodies and antigen-binding fragments thereof. For example, it has been shown that V_{HH} domains and Nanobodies can inhibit enzymes (see for example WO 97/49805; Transue et al., (1998), supra; and Lauwereys et al., (1998), supra).

As mentioned above, the invention generally relates to Nanobodies directed against A-beta, as well as to polypeptides comprising or essentially consisting of one or more of such Nanobodies, that can be used for the prophylactic, therapeutic and/or diagnostic purposes described herein.

As also further described herein, the invention further relates to nucleic acids encoding such Nanobodies and polypeptides, to methods for preparing such Nanobodies and polypeptides, to host cells expressing or capable of expressing such Nanobodies or polypeptides, to compositions comprising such Nanobodies, polypeptides, nucleic acids or host cells, and to uses of such Nanobodies, polypeptides, nucleic acids, host cells or compositions.

Generally, it should be noted that the term Nanobody as used herein in its broadest sense is not limited to a specific biological source or to a specific method of preparation. For example, as will be discussed in more detail below, the Nanobodies of the invention can generally be obtained: (1) by isolating the V_{HH} domain of a naturally occurring heavy chain antibody; (2) by expression of a nucleotide sequence encoding a naturally occurring V_{HH} domain; (3) by “humanization” (as described herein) of a naturally occurring V_{HH} domain or by expression of a nucleic acid encoding a such humanized V_{HH} domain; (4) by “camelization” (as described herein) of a naturally occurring V_{H} domain from any animal species, and in particular a from species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. Suitable methods and techniques for performing the foregoing will be clear to the skilled person based on the disclosure herein and for example include the methods and techniques described in more detail herein.

One preferred class of Nanobodies corresponds to the V_{HH} domains of naturally occurring heavy chain antibodies directed against A-beta. As further described herein, such V_{HH} sequences can generally be generated or obtained by suitably immunizing a species of Camelid with A-beta (i.e. so as to raise an immune response and/or heavy chain antibodies directed against A-beta), by obtaining a suitable biological sample from said Camelid (such as a blood sample, serum sample or sample of B-cells), and by generating V_{HH} sequences directed against A-beta starting from said sample, using any suitable technique known per se. Such techniques will be clear to the skilled person and/or are further described herein.

Alternatively, such naturally occurring V_{HH} domains against A-beta can be obtained from naïve libraries of Camelid V_{HH} sequences, for example by screening such a library using A-beta or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve V_{HH} libraries by techniques such as random mutagenesis and/or CDR shuffling, as for example described in WO 00/43507.

Yet another technique for obtaining V_{HH} sequences directed against A-beta involves suitably immunizing a transgenic mammal that is capable of expressing heavy chain antibodies (i.e. so as to raise an immune response and/or heavy chain antibodies directed against A-beta), obtaining a suitable biological sample from said transgenic mammal (such as a blood sample, serum sample or sample of B-cells), and then generating V_{HH} sequences directed against A-beta starting from said sample, using any suitable technique known per se. For example, for this purpose, the heavy chain antibody-expressing mice and the further methods and techniques described in WO 02/085945 and in WO 04/049794 can be used.

A particularly preferred class of Nanobodies of the invention comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized Nanobodies of the invention can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another particularly preferred class of Nanobodies of the invention comprises Nanobodies with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized Nanobody is preferably a V_{H} sequence from a mammal, more preferably the V_{H} sequence of a human being, such as a V_{H}3 sequence. However, it should be noted that such camelized Nanobodies of the invention can be obtained in any suitable manner known per se (i.e. as indicated under points (1)-(8) above) and thus are not strictly limited to polypeptides that have been obtained using a polypeptide that comprises a naturally occurring V_{H} domain as a starting material.

For example, again as further described herein, both “humanization” and “camelization” can be performed by providing a nucleotide sequence that encodes a naturally occurring V_{HH} domain or V_{H} domain, respectively, and then changing, in a manner known per se, one or more codons in said nucleotide sequence in such a way that the new nucleotide sequence encodes a “humanized” or “camelized” Nanobody of the invention, respectively. This nucleic acid can then be expressed in a manner known per se, so as to provide the desired Nanobody of the invention. Alternatively, based on the amino acid sequence of a naturally occurring V_{HH} domain or V_{H} domain, respectively, the amino acid sequence of the desired humanized or camelized Nanobody of the invention, respectively, can be designed and then synthesized de novo using techniques for peptide synthesis known per se. Also, based on the amino acid sequence or nucleotide sequence of a naturally occurring V_{HH} domain or V_{H} domain, respectively, a nucleotide sequence encoding the desired humanized or camelized Nanobody of the invention, respectively, can be designed and then synthesized de novo using techniques for nucleic acid synthesis known per se, after which the nucleic acid thus obtained can be expressed in a manner known per se, so as to provide the desired Nanobody of the invention.

Other suitable methods and techniques for obtaining the Nanobodies of the invention and/or nucleic acids encoding the same, starting from naturally occurring V_{H} sequences or preferably V_{HH} sequences, will be clear from the skilled person, and may for example comprise combining one or more parts of one or more naturally occurring V_{H} sequences (such as one or more FR sequences and/or CDR sequences), one or more parts of one or more naturally occurring V_{HH} sequences (such as one or more FR sequences or CDR sequences), and/or one or more synthetic or semi-synthetic sequences, in a suitable manner, so as to provide a Nanobody of the invention or a nucleotide sequence or nucleic acid encoding the same.

According to one preferred, but non-limiting aspect of the aspect of the invention, a Nanobody in its broadest sense can be generally defined as a polypeptide comprising:
(a) an amino acid sequence that is comprised of four framework regions/sequences interrupted by three complementarity determining regions/sequences, in which the amino acid residue at position 108 according to the Kabat numbering is Q; and/or:
(b) an amino acid sequence that is comprised of four framework regions/sequences interrupted by three complementarity determining regions/sequences, in which the amino acid residue at position 45 according to the Kabat numbering is a charged amino acid (as defined herein) or a cysteine residue, and position 44 is preferably an E;
   and/or:
(c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, in a first preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q; and/or in which:
(b) the amino acid residue at position 45 according to the Kabat numbering is a charged amino acid or a cysteine and the amino acid residue at position 44 according to the Kabat numbering is preferably E;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, a Nanobody in its broadest sense can be generally defined as a polypeptide comprising:
(a) an amino acid sequence that is comprised of four framework regions/sequences interrupted by three complementarity determining regions/sequences, in which the amino acid residue at position 108 according to the Kabat numbering is Q; and/or:
(b) an amino acid sequence that is comprised of four framework regions/sequences interrupted by three complementarity determining regions/sequences, in which the amino acid residue at position 44 according to the Kabat numbering is E and in which the amino acid residue at position 45 according to the Kabat numbering is an R; and/or:
(c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, according to a preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(e) the amino acid residue at position 108 according to the Kabat numbering is Q;
   and/or in which:
(f) the amino acid residue at position 44 according to the Kabat numbering is E and in which the amino acid residue at position 45 according to the Kabat numbering is an R; and/or in which:
(g) the amino acid residue at position 103 according to the Kabat numbering is chosen from the group consisting of P, R and S, and is in particular chosen from the group consisting of R and S; and in which:
(h) CDR1, CDR2 and CDR3 are as defined herein, and are preferably as defined according to one of the preferred embodiments herein, and are more preferably as defined according to one of the more preferred embodiments herein.

In particular, a Nanobody against A-beta according to the invention may have the structure:

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q;
   and/or in which:
(b) the amino acid residue at position 44 according to the Kabat numbering is E and in which the amino acid residue at position 45 according to the Kabat numbering is an R;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, according to one preferred, but non-limiting aspect of the aspect of the invention, a Nanobody can generally be defined as a polypeptide comprising an amino acid sequence that is comprised of four framework regions/sequences interrupted by three complementarity determining regions/sequences, in which;
(a-1) the amino acid residue at position 44 according to the Kabat numbering is chosen from the group consisting of A, G, E, D, G, Q, R, S, L; and is preferably chosen from the group consisting of G, E or Q; and
(a-2) the amino acid residue at position 45 according to the Kabat numbering is chosen from the group consisting of L, R or C; and is preferably chosen from the group consisting of L or R; and
(a-3) the amino acid residue at position 103 according to the Kabat numbering is chosen from the group consisting of W, R or S; and is preferably W or R, and is most preferably W;
(a-4) the amino acid residue at position 108 according to the Kabat numbering is Q;
   or in which:
(b-1) the amino acid residue at position 44 according to the Kabat numbering is chosen from the group consisting of E and Q; and
(b-2) the amino acid residue at position 45 according to the Kabat numbering is R; and
(b-3) the amino acid residue at position 103 according to the Kabat numbering is chosen from the group consisting of W, R and S; and is preferably W;
(b-4) the amino acid residue at position 108 according to the Kabat numbering is chosen from the group consisting of Q and L; and is preferably Q;
   or in which:
(c-1) the amino acid residue at position 44 according to the Kabat numbering is chosen from the group consisting of A, G, E, D, Q, R, S and L; and is preferably chosen from the group consisting of G, E and Q; and
(c-2) the amino acid residue at position 45 according to the Kabat numbering is chosen from the group consisting of L, R and C; and is preferably chosen from the group consisting of L and R; and
(c-3) the amino acid residue at position 103 according to the Kabat numbering is chosen from the group consisting of P, R and S; and is in particular chosen from the group consisting of R and S; and
(c-4) the amino acid residue at position 108 according to the Kabat numbering is chosen from the group consisting of Q and L; is preferably Q;
   and in which
(d) CDR1, CDR2 and CDR3 are as defined herein, and are preferably as defined according to one of the preferred embodiments herein, and are more preferably as defined according to one of the more preferred embodiments herein.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of A, G, E, D, G, Q, R, S, L; and is preferably chosen from the group consisting of G, E or Q;
   and in which:
(b) the amino acid residue at position 45 according to the Kabat numbering is chosen from the group consisting of L, R or C; and is preferably chosen from the group consisting of L or R;
   and in which:
(c) the amino acid residue at position 103 according to the Kabat numbering is chosen from the group consisting of W, R or S; and is preferably W or R, and is most preferably W;
   and in which
(d) the amino acid residue at position 108 according to the Kabat numbering is Q;
   and in which:
(e) CDR1, CDR and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of E and Q;
   and in which:
(b) the amino acid residue at position 45 according to the Kabat numbering is R;
   and in which:
(c) the amino acid residue at position 103 according to the Kabat numbering is chosen from the group consisting of W, R and S; and is preferably W;
   and in which:
(d) the amino acid residue at position 108 according to the Kabat numbering is Q;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of A, G, E, D, Q, R, S and L; and is preferably chosen from the group consisting of G, E and Q;
   and in which:
(b) the amino acid residue at position 45 according to the Kabat numbering is chosen from the group consisting of L, R and C; and is preferably chosen from the group consisting of L and R;
   and in which:
(c) the amino acid residue at position 103 according to the Kabat numbering is chosen from the group consisting of P, R and S; and is in particular chosen from the group consisting of R and S;
   and in which:
(d) the amino acid residue at position 108 according to the Kabat numbering is chosen from the group consisting of Q and L; is preferably Q;
   and in which:
(e) CDR1, CDR2 and CDR3 are as defined herein, and are preferably as defined according to one of the preferred embodiments herein, and are more preferably as defined according to one of the more preferred embodiments herein.

Two particularly preferred, but non-limiting groups of the Nanobodies of the invention are those according to a) above; according to (a-1) to (a-4) above; according to b) above; according to (b-1) to (b-4) above; according to (c) above; and/or according to (c-1) to (c-4) above, in which;
a) the amino acid residues at positions 44-47 according to the Kabat numbering form the sequence GLEW (or a GLEW-like sequence as defined herein) and the amino acid residue at position 108 is Q;
   or in which:
b) the amino acid residues at positions 43-46 according to the Kabat numbering form the sequence KERE or KQRE (or a KERE-like sequence) and the amino acid residue at position 108 is Q or L, and is preferably Q.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residues at positions 44-47 according to the Kabat numbering form the sequence GLEW (or a GLEW-like sequence as defined herein) and the amino acid residue at position 108 is Q;
   and in which:
(b) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residues at positions 43-46 according to the Kabat numbering form the sequence KERE or KQRE (or a KERE-like sequence) and the amino acid residue at position 108 is Q or L, and is preferably Q;
   and in which:
(b) CDR1, CDR2 and CDR3 are as defined herein, and are preferably as defined according to one of the preferred embodiments herein, and are more preferably as defined according to one of the more preferred embodiments herein.

In the Nanobodies of the invention in which the amino acid residues at positions 43-46 according to the Kabat numbering form the sequence KERE or KQRE, the amino acid residue at position 37 is most preferably F. In the Nanobodies of the invention in which the amino acid residues at positions 44-47 according to the Kabat numbering form the sequence GLEW, the amino acid residue at position 37 is chosen from the group consisting of Y, H, I, L, V or F, and is most preferably F.

Thus, without being limited hereto in any way, on the basis of the amino acid residues present on the positions mentioned above, the Nanobodies of the invention can generally be classified is on the basis of the following three groups:
a) The “GLEW-group”: Nanobodies with the amino acid sequence GLEW at positions 44-47 according to the Kabat numbering and Q at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table A-3 below;
b) The “KERE-group”: Nanobodies with the amino acid sequence KERE or KQRE or at positions 43-46 according to the Kabat numbering and Q or L at position 108 according to the Kabat numbering. As further described herein, Nanobodies within this group usually have a F at position 37, an L or F at position 47; and can have a W, P, R or S at position 103, and preferably have a W at position 103;
c) The “103 P, R, S-group”: Nanobodies with a P, R or S at position 103. These Nanobodies can have either the amino acid sequence GLEW at positions 44-47 of the Kabat numbering or the amino acid sequence KERE or KQRE at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Thus, in another preferred, but non-limiting aspect, a Nanobody of the invention may be a Nanobody belonging to the GLEW-group (as defined herein), and in which CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may be a Nanobody belonging to the KERE-group (as defined herein), and CDR1, CDR2 and CDR3 are as defined herein, and are preferably as defined according to one of the preferred embodiments herein, and are more preferably as defined according to one of the more preferred embodiments herein.

Thus, in another preferred, but non-limiting aspect, a Nanobody of the invention may be a Nanobody belonging to the 103 P, R, S-group (as defined herein), and in which CDR1, CDR2 and CDR3 are as defined herein, and are preferably as defined according to one of the preferred embodiments herein, and are more preferably as defined according to one of the more preferred embodiments herein.

Also, more generally and in addition to the 108Q, 43E/44R and 103P,R,S residues mentioned above, the Nanobodies of the invention can contain, at one or more positions that in a conventional V_{H} domain would form (part of) the V_{H}/V_{L} interface, one or more amino acid residues that are more highly charged than the amino acid residues that naturally occur at the same position(s) in the corresponding naturally occurring V_{H} sequence, and in particular one or more charged amino acid residues (as mentioned in Table A-2). Such substitutions include, but are not limited to, the GLEW-like sequences mentioned in Table A-3 below; as well as the substitutions that are described in the International Application WO 00/29004 for so-called “microbodies”, e.g. so as to obtain a Nanobody with Q at position 108 in combination with KLEW at positions 44-47. Other possible substitutions at these positions will be clear to the skilled person based upon the disclosure herein.

In one embodiment of the Nanobodies of the invention, the amino acid residue at position 83 is chosen from the group consisting of L, M, S, V and W; and is preferably L.

Also, in one embodiment of the Nanobodies of the invention, the amino acid residue at position 83 is chosen from the group consisting of R, K, N, E, G, I, T and Q; and is most preferably either K or E (for Nanobodies corresponding to naturally occurring V_{HH} domains) or R (for “humanized” Nanobodies, as described herein). The amino acid residue at position 84 is chosen from the group consisting of P, A, R, S, D T, and V in one embodiment, and is most preferably P (for Nanobodies corresponding to naturally occurring V_{HH} domains) or R (for “humanized” Nanobodies, as described herein).

Furthermore, in one embodiment of the Nanobodies of the invention, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the “Hallmark Residues”. The Hallmark Residues and the amino acid residues at the corresponding positions of the most closely related human V_{H} domain, V_{H}3, are summarized in Table A-3.

Some especially preferred but non-limiting combinations of these Hallmark Residues as occur in naturally occurring V_{HH} domains are mentioned in Table A-4. For comparison, the corresponding amino acid residues of the human V_{H}3 called DP-47 have been indicated in italics.

TABLE A-2
Hallmark Residues in Nanobodies

| Position | Human V_{H}3 | Hallmark Residues |
|---|---|---|
| 11 | L, V; | L, M, S, V, W; preferably L |
| | predominantly L | |
| 37 | V, I, F; usually V | F⁽¹⁾, Y, H, I, L or V, preferably F⁽¹⁾ or Y |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁸⁾ | L | L⁽²⁾, R⁽³⁾, C, I, L, P, Q, V; preferably L⁽²⁾ |
| | | or R⁽³⁾ |
| 47⁽⁸⁾ | W, Y | W⁽²⁾, L⁽¹⁾ or F⁽¹⁾, A, G, I, M, R, S, V or |
| | | Y; preferably W⁽²⁾, L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; | P⁽⁵⁾, A, L, R, S, T, D, V; preferably P |
| | predominantly A | |
| 103 | W | W⁽⁴⁾, P⁽⁶⁾, R⁽⁶⁾, S; preferably W |
| 104 | G | G or D; preferably G |
| 108 | L, M or T; | Q, L⁽⁷⁾ or R; preferably Q or L⁽⁷⁾ |
| | predominantly L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE A-3
Some preferred but non-limiting combinations of Hallmark Residues in
naturally occurring Nanobodies.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |

For humanization of these combinations, reference is made to the specification.

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables −A4 to A7 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering Chothia (supra) that the residues at these positions already form part of CDR1.)

In Tables A4 to A7, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

For reference only, Table A-5 also contains data on the V_{HH} entropy (“V_{HH} Ent.”) and V_{HH} variability (“V_{HH} Var.”) at each amino acid position for a representative sample of 1118 V_{HH} sequences (data kindly provided by David Lutje Hulsing and Prof. Theo Verrips of Utrecht University). The values for the V_{HH} entropy and the V_{HH} variability provide a measure for the variability and degree of conservation of amino acid residues between the 1118 V_{HH} sequences analyzed: low values (i.e. <1, such as <0.5) indicate that an amino acid residue is highly conserved between the V_{HH} sequences (i.e. little variability). For example, the G at position 8 and the G at position 9 have values for the V_{HH} entropy of 0.1 and 0 respectively, indicating that these residues are highly conserved and have vary little variability (and in case of position 9 is G in all 1118 sequences analysed), whereas for residues that form part of the CDR's generally values of 1.5 or more are found (data not shown). Note that (1) the amino acid residues listed in the second column of Table A-5 are based on a bigger sample than the 1118 V_{HH} sequences that were analysed for determining the V_{HH} entropy and V_{HH} variability referred to in the last two columns; and (2) the data represented below supports the hypothesis that the amino acid residues at positions 27-30 and maybe even also at positions 93 and 94 already form part of the CDR's (although the invention is not limited to any specific hypothesis or explanation, and as mentioned above, herein the numbering according to Kabat is used). For a general explanation of sequence entropy, sequence variability and the methodology for determining the same, see Oliveira et al., PROTEINS: Structure, Function and Genetics, 52: 544-552 (2003).

TABLE A-4
Non-limiting examples of amino acid residues in FR1 (for the
footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, | | 0.8 | 2 |
| | V, W; preferably L | | | |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, Y | 1.7 | 11 |
| 29 | F, V | F, L, D, S, I, G, V, A | 1.9 | 11 |
| 30 | S, D, G | N, S, E, G, A, D, M, T | 1.8 | 11 |

TABLE A-5
Non-limiting examples of amino acid residues in FR2
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or | | 1.1 | 6 |
| | V, preferably F⁽¹⁾ or Y | | | |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, | | 1.3 | 5 |
| | R, S, L; preferably G⁽²⁾, E⁽³⁾ or | | | |
| | Q; most preferably G⁽²⁾ or E⁽³⁾. | | | |
| 45 | Hallmark residue: L⁽²⁾, R⁽³⁾, C, I, L, | | 0.6 | 4 |
| | P, Q, V; preferably L⁽²⁾ or R⁽³⁾ | | | |
| 46 | E, V | E, D, K, Q, V | 0.4 | 2 |
| 47 | Hallmark residue: W⁽²⁾, L⁽¹⁾ or F⁽¹⁾, | | 1.9 | 9 |
| | A, G, I, M, R, S, V or Y; preferably W⁽²⁾, | | | |
| | L⁽¹⁾, F⁽¹⁾ or R | | | |
| 48 | V | V, I, L | 0.4 | 3 |
| 49 | S, A, G | A, S, G, T, V | 0.8 | 3 |

TABLE A-6
Non-limiting examples of amino acid residues in FR3
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, | | 0.9 | 7 |
| | Q or T; preferably K or R; most preferably K | | | |
| 84 | Hallmark residue: P⁽⁵⁾, A, D, L, | | 0.7 | 6 |
| | R, S, T, V; preferably P | | | |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE A-7
Non-limiting examples of amino acid residues in FR4
(for the footnotes, see the footnotes to Table A-3)

| | | |
|---|---|---|
| Amino acid residue(s): | V_{HH} | V_{HH} |

| | | | | |
|---|---|---|---|---|
| Pos. | Human V_{H}3 | Camelid V_{HH}'s | Ent. | Var. |

| | | | |
|---|---|---|---|
| 103 | Hallmark residue: W⁽⁴⁾, P⁽⁶⁾, | 0.4 | 2 |
| | R⁽⁶⁾, S; preferably W | | |
| 104 | Hallmark residue: G or D; | 0.1 | 1 |
| | preferably G | | |

| | | | | |
|---|---|---|---|---|
| 105 | Q, R | Q, E, K, P, R | 0.6 | 4 |
| 106 | G | G | 0.1 | 1 |
| 107 | T | T, A, I | 0.3 | 2 |

| | | | |
|---|---|---|---|
| 108 | Hallmark residue: Q, | 0.4 | 3 |
| | L⁽⁷⁾ or R; preferably Q or L⁽⁷⁾ | | |

| | | | | |
|---|---|---|---|---|
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

Thus, in another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the Hallmark residues are as defined herein;
   and in which:
(b) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: and in which
(a) FR1 is chosen from the group consisting of the amino acid sequence:

| | |
|---|---|
| [1] QVQLQESGGGXYQAGGSLRLSCAASG [26] | [SEQ ID NO: 1] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s);
   and in which:
(b) FR2 is chosen from the group consisting of the amino acid sequence:

| | |
|---|---|
| [36] WXRQAPGKXXEXVA [49] | [SEQ ID NO: 2] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s);
   and in which:
(c) FR3 is chosen from the group consisting of the amino acid sequence:

[SEQ ID NO: 3]

[66] RFTISRDNAKNTVYLQMNSLXXEDTAVYYCAA [94]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s);
   and in which:
(d) FR4 is chosen from the group consisting of the amino acid sequence:

| | |
|---|---|
| [103] XXQGTXVTVSS [113] | [SEQ ID NO: 4] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s);
   and in which:
(e) CDR1, CDR2 and CDR3 are as defined herein, and are preferably as defined according to one of the preferred embodiments herein, and are more preferably as defined according to one of the more preferred embodiments herein;
   in which the Hallmark Residues are indicated by “X” and are as defined hereinabove and in which the numbers between brackets refer to the amino acid positions according to the Kabat numbering.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

| | |
|---|---|
| [1] QVQLQESGGGLVQAGGSLRLSCAASG [26] | [SEQ ID NO: 5] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and
no amino acid deletions or insertions, compared to the above amino acid sequence(s); and
iii) the Hallmark residue at position is as indicated in the sequence above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above;
   and in which:
(b) FR2 is chosen from the group consisting of the amino acid sequences:

| | |
|---|---|
| [36] WFRQAPGKERELVA [49] | [SEQ ID NO: 6] |
| [36] WFRQAPGKEREFVA [49] | [SEQ ID NO: 7] |
| [36] WFRQAPGKEREGA [49] | [SEQ ID NO: 8] |
| [36] WFRQAPGKQRELVA [49] | [SEQ ID NO: 9] |
| [36] WFRQAPGKQREFVA [49] | [SEQ ID NO: 10] |
| [36] WYRQAPGKGLEWA [49] | [SEQ ID NO: 11] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 12]

[66] RFTISRDNAKNTVYLQMNSLKPEDTAVYYCAA [94]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequences:

| | |
|---|---|
| [103] WGQGTQVTVSS [113] | [SEQ ID NO: 13] |
| [103] WGQGTLVTVSS [113] | [SEQ ID NO: 14] |

or from the group consisting of amino acid sequences that have at least 80%, preferably at least 90%, more preferably at least 95%, even more preferably at least 99% sequence identity (as defined herein) with one of the above amino acid sequence; in which
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

| | |
|---|---|
| [1] QVQLQESGGGLVQAGGSLRLSCAASG [26] | [SEQ ID NO: 5] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above;
   and in which:
(b) FR2 is chosen from the group consisting of the amino acid sequences:

| | |
|---|---|
| [36] WFRQAPGKERELVA [49] | [SEQ ID NO: 6] |
| [36] WFRQAPGKEREFVA [49] | [SEQ ID NO: 7] |
| [36] WFRQAPGKEREGA [49] | [SEQ ID NO: 8] |
| [36] WFRQAPGKQRELVA [49] | [SEQ ID NO: 9] |
| [36] WFRQAPGKQREFVA [49] | [SEQ ID NO: 10] |

and/or from the group consisting of amino acid sequences that have 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 12]

[66] RFTISRDNAKNTVYLQMNSLKPEDTAVYYCAA [94]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequences:

| | |
|---|---|
| [103] WGQGTQVTVSS [113] | [SEQ ID NO: 13] |
| [103] WGQGTLVTVSS [113] | [SEQ ID NO: 14] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the amino acid sequence:

| | |
|---|---|
| [1] QVQLQESGGGLVQAGGSLRLSCAASG [26] | [SEQ ID NO: 5] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position is as indicated in the sequence above;
   and in which:
(b) FR2 is chosen from the group consisting of the amino acid sequence:

| | |
|---|---|
| [36] WYRQAPGKGLEWA [49] | [SEQ ID NO: 11] |

and/or from the group consisting of amino acid sequences that have 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 12]

[66] RFTISRDNAKNTVYLQMNSLKPEDTAVYYCAA [94]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequence:

| | |
|---|---|
| [103] WGQGTQVTVSS [113] | [SEQ ID NO: 13] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

Some other framework sequences that can be present in the Nanobodies of the invention can be found in the European patent EP 656 946 mentioned above (see for example also the granted US equivalent U.S. Pat. No. 5,759,808),

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
and in which
(a) FR1 is chosen from the group consisting of the FR1 sequences present in the Nanobodies of SEQ ID NO's: 73-105, and in particular from the group consisting of the FR1 sequences present in the humanized Nanobodies of SEQ ID NO's: 85-105,
(b) or from the group consisting of amino acid sequences that have at least 80%, preferably at least 90%, more preferably at least 95%, even more preferably at least 99% sequence identity (as defined herein) with one of said FR1 sequences; in which
   i) any amino acid substitution at any position other than a Hallmark position is preferably either a conservative amino acid substitution (as defined herein) and/or an amino acid substitution as defined in Table A-5; and/or
   ii) said amino acid sequence preferably only contains amino acid substitutions, and no amino acid deletions or insertions, compared to said FR1 sequence; and
   iii) the Hallmark residue at position is as indicated in said FR1 sequence;
   and/or from the group consisting of amino acid sequences that have 3, 2 or only 1 “amino acid difference(s)” (as defined herein) with one of said FR1 sequences, in which:
   i) any amino acid substitution at any position other than a Hallmark position is preferably either a conservative amino acid substitution (as defined herein) and/or an amino acid substitution as defined in Table A-5; and/or
   ii) said amino acid sequence preferably only contains amino acid substitutions, and no amino acid deletions or insertions, compared to said FR1 sequence; and
   iii) the Hallmark residue at position is as indicated in said FR1 sequence;
      and in which:
(c) FR2 is chosen from the group consisting of the FR2 sequences present in the Nanobodies of SEQ ID NO's: 73-105, and in particular from the group consisting of the FR2 sequences present in the humanized Nanobodies of SEQ ID NO's: 85-105, or from the group consisting of amino acid sequences that have at least 80%, preferably at least 90%, more preferably at least 95%, even more preferably at least 99% sequence identity (as defined herein) with one of said FR2 sequences; in which
   i) any amino acid substitution at any position other than a Hallmark position is preferably either a conservative amino acid substitution (as defined herein) and/or an amino acid substitution as defined in Table A-6; and/or
   ii) said amino acid sequence preferably only contains amino acid substitutions, and no amino acid deletions or insertions, compared to said FR2 sequence; and
   iii) the Hallmark residues at positions 37, 44, 45 and 47 are as indicated in said FR2 sequence;
   and/or from the group consisting of amino acid sequences that have 3, 2 or only 1 “amino acid difference(s)” (as defined herein) with one of said FR2 sequences, in which:
   i) any amino acid substitution at any position other than a Hallmark position is preferably either a conservative amino acid substitution (as defined herein) and/or an amino acid substitution as defined in Table A-6; and/or
   ii) said amino acid sequence preferably only contains amino acid substitutions, and no amino acid deletions or insertions, compared to said FR2 sequence; and
   iii) the Hallmark residues at positions 37, 44, 45 and 47 are as indicated in said FR2 sequence;
      and in which:
(d) FR3 is chosen from the group consisting of the FR3 sequences present in the Nanobodies of SEQ ID NO's: 73-105, and in particular from the group consisting of the FR3 sequences present in the humanized Nanobodies of SEQ ID NO's: 85-105, or from the group consisting of amino acid sequences that have at least 80%, preferably at least 90%, more preferably at least 95%, even more preferably at least 99% sequence identity (as defined herein) with one of said FR3 sequences; in which
   i) any amino acid substitution at any position other than a Hallmark position is preferably either a conservative amino acid substitution (as defined herein) and/or an amino acid substitution as defined in Table A-7; and/or
   ii) said amino acid sequence preferably only contains amino acid substitutions, and no amino acid deletions or insertions, compared to said FR3 sequence; and
   iii) the Hallmark residues at positions 83 and 84 are as indicated in said FR3 sequence;
   and/or from the group consisting of amino acid sequences that have 3, 2 or only 1 “amino acid difference(s)” (as defined herein) with one of said FR3 sequences, in which:
   i) any amino acid substitution at any position other than a Hallmark position is preferably either a conservative amino acid substitution (as defined herein) and/or an amino acid substitution as defined in Table A-7; and/or
   ii) said amino acid sequence preferably only contains amino acid substitutions, and no amino acid deletions or insertions, compared to said FR3 sequence; and
   iii) the Hallmark residues at positions 83 and 84 are as indicated in said FR3 sequence;
      and in which:
(e) FR4 is chosen from the group consisting of the FR4 sequences present in the Nanobodies of SEQ ID NO's: 73-105, and in particular from the group consisting of the FR4 sequences present in the humanized Nanobodies of SEQ ID NO's: 85-105, or from the group consisting of amino acid sequences that have at least 80%, preferably at least 90%, more preferably at least 95%, even more preferably at least 99% sequence identity (as defined herein) with one of said FR4 sequences; in which
   i) any amino acid substitution at any position other than a Hallmark position is preferably either a conservative amino acid substitution (as defined herein) and/or an amino acid substitution as defined in Table A-8; and/or
   ii) said amino acid sequence preferably only contains amino acid substitutions, and no amino acid deletions or insertions, compared to said FR4 sequence; and
   iii) the Hallmark residues at positions 103, 104 and 108 are as indicated in said FR3 sequence;
   and/or from the group consisting of amino acid sequences that have 3, 2 or only 1 “amino acid difference(s)” (as defined herein) with one of said FR4 sequences, in which:
   i) any amino acid substitution at any position other than a Hallmark position is preferably either a conservative amino acid substitution (as defined herein) and/or an amino acid substitution as defined in Table A-8; and/or
   ii) said amino acid sequence preferably only contains amino acid substitutions, and no amino acid deletions or insertions, compared to said FR4 sequence; and
   iii) the Hallmark residues at positions 103, 104 and 108 are as indicated in said FR4 sequence;
      and in which:
(f) CDR1, CDR2 and CDR3 are as defined herein, and are preferably as defined according to one of the preferred embodiments herein, and are more preferably as defined according to one of the more preferred embodiments herein.

Some particularly preferred Nanobodies of the invention can be chosen from the group consisting of the amino acid sequences of SEQ ID NO's 73-105, and in particular in the humanized Nanobodies of SEQ ID NO's 85-105 or from the group consisting of amino acid sequences that have at least 80%, preferably at least 90%, more preferably at least 95%, even more preferably at least 99% sequence identity (as defined herein) with one of the amino acid sequences of SEQ ID NO's 73-105 (and preferably of SEQ ID NO's 85 to 105); in which
i) the Hallmark residues can be as indicated in Table A-3 above;
ii) any amino acid substitution at any position other than a Hallmark position is preferably either a conservative amino acid substitution (as defined herein) and/or an amino acid substitution as defined in Tables 5-8; and/or
iii) said amino acid sequence preferably only contains amino acid substitutions, and no amino acid deletions or insertions, compared to the above amino acid sequence(s).

Some even more particularly preferred Nanobodies of the invention can be chosen from the group consisting of the amino acid sequences of SEQ ID NO's 73-105, and in particular in the humanized Nanobodies of SEQ ID NO's 85-105 or from the group consisting of amino acid sequences that have at least 80%, preferably at least 90%, more preferably at least 95%, even more preferably at least 99% sequence identity (as defined herein) with one of the amino acid sequences of SEQ ID NO's 73-105 (and preferably of SEQ ID NO's 85-105); in which
(1) the Hallmark residues are as indicated in the pertinent sequence chosen from SEQ ID NO's 73-105 (and preferably from SEQ ID NO's 85-105);
(2) any amino acid substitution at any position other than a Hallmark position is preferably either a conservative amino acid substitution (as defined herein) and/or an amino acid substitution as defined in Tables 5-8; and/or
(3) said amino acid sequence preferably only contains amino acid substitutions, and no amino acid deletions or insertions, compared to the pertinent sequence chosen from SEQ ID NO's 73-105 (and preferably from SEQ ID NO's 85-105).

Some of the most preferred Nanobodies of the invention can be chosen from the group consisting of the amino acid sequences of SEQ ID NO's 73-105 and SEQ ID NO's 85-105, and in particular from the humanized Nanobodies of SEQ ID NO's 85-105.

Preferably, the CDR sequences and FR sequences in the Nanobodies of the invention are such that the Nanobody of the invention binds to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the Nanobody of the invention against A-beta can be determined in a manner known per se, for example using the assay described herein.

According to one non-limiting aspect of the invention, a Nanobody may be as defined herein, but with the proviso that it has at least “one amino acid difference” (as defined herein) in at least one of the framework regions compared to the corresponding framework region of a naturally occurring human V_{H} domain, and in particular compared to the corresponding framework region of DP-47. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring human V_{H} domain, and in particular compared to the corresponding framework region of DP-47. Usually, a Nanobody will have at least one such amino acid difference with a naturally occurring V_{H} domain in at least one of FR2 and/or FR4, and in particular at least one of the Hallmark residues in FR2 and/or FR4 (again, (including those at positions 108, 103 and/or 45).

Also, a humanized Nanobody of the invention may be as defined herein, but with the proviso that it has at least “one amino acid difference” (as defined herein) in at least one of the framework regions compared to the corresponding framework region of a naturally occurring V_{HH} domain. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring V_{HH} domain. Usually, a Nanobody will have at least one such amino acid difference with a naturally occurring V_{HH} domain in at least one of FR2 and/or FR4, and in particular at least one of the Hallmark residues in FR2 and/or FR4 (again, (including those at positions 108, 103 and/or 45).

One embodiment of the present invention is a polypeptide comprising at least one heavy chain antibody, or a functional fragment thereof (including humanized functional fragments thereof), directed against A-beta.

Another embodiment of the present invention is a polypeptide as defined above, wherein at least one heavy chain antibody, or a functional fragment thereof, directed against A-beta is a Nanobody™, or a functional fragment thereof.

Another embodiment of the present invention is a polypeptide as defined above, wherein at least one heavy chain antibody, or a functional fragment thereof, corresponds to a sequence represented by any of SEQ ID NOs: 73-105, preferably 85-105.

Another embodiment of the present invention is a polypeptide as defined above wherein the number of Nanobodies, or functional fragments thereof, directed against A-beta is at least two.

Another embodiment of the present invention is a polypeptide as defined above, further comprising at least one heavy chain antibody, or a functional fragment thereof, directed to improving the half-life of the polypeptide in vivo.

Another embodiment of the present invention is a polypeptide as defined above wherein said heavy chain antibody, or a functional fragment thereof, directed to improving the half-life is a heavy chain antibody, or a functional fragment thereof, directed against a serum protein.

Another embodiment of the present invention is a polypeptide as defined above wherein at least one heavy chain antibody, or a functional fragment thereof, is capable of clearance of amyloid plaque from the brain or other parts in the body.

Another embodiment of the present invention is a polypeptide as defined above wherein at least one heavy chain antibody, or a functional fragment thereof, is capable of inhibiting the interaction between A-beta and another A-beta.

Another embodiment of the present invention is a polypeptide as defined above wherein one or more amino acids of at least one heavy chain antibody, or a functional fragment thereof, have been substituted without substantially altering the antigen binding capacity.

Another embodiment of the present invention is a polypeptide as defined above, wherein at least one heavy chain antibody or nanobody is a homologous sequence, a functional portion, or a functional portion of a homologous sequence of the full length heavy chain antibody or nanobody.

Another embodiment of the present invention is a polypeptide as defined above wherein at least one heavy chain antibody, or a functional fragment thereof, is capable of binding to a neo-epitope created or exposed following a secretase mediated cleavage of APP and APLP, or any other cleavage resulting in an A-beta cleavage product.

Another embodiment of the present invention is a polypeptide as defined above corresponding to a sequence represented by any of SEQ ID NOs: 117-183.

Another embodiment of the present invention is a polypeptide as defined above wherein at least one heavy chain antibody, or a functional fragment thereof, directed to improving the half-life is modified by pegylation.

Another embodiment of the present invention is a polypeptide as defined above wherein said heavy chain antibody, or a functional fragment thereof, directed against a serum protein is a Nanobody, or a functional fragment thereof.

Another embodiment of the present invention is a polypeptide as defined above wherein said serum protein is any of serum albumin, serum immunoglobulins, thyroxine-binding protein, transferrin or fibrinogen.

Another embodiment of the present invention is a polypeptide as defined above wherein said heavy chain antibody, or a functional fragment thereof, directed against a serum protein or nanobody, or a functional fragment thereof, is humanized.

Another embodiment of the present invention is a polypeptide as defined above wherein a serum protein is a fragment of a serum protein.

Another embodiment of the present invention is a polypeptide as defined above further comprising a heavy chain antibody, or a functional fragment thereof, directed against protein tau.

Another embodiment of the present invention is a polypeptide as defined above wherein said heavy chain antibody, or a functional fragment thereof, directed against protein tau is a Nanobody.

Another embodiment of the present invention is a polypeptide as defined above wherein said heavy chain antibody or nanobody, or a functional fragment thereof, directed against protein tau humanized.

Another embodiment of the present invention is a polypeptide as defined above wherein protein tau is a fragment of protein tau.

Another embodiment of the present invention is a polypeptide as defined above, further comprising one or more linker sequences.

Another embodiment of the present invention is a polypeptide as defined above wherein said A-beta is a fragment of A-beta.

Another embodiment of the present invention is a polypeptide as defined above wherein at least one heavy chain antibody, or a functional fragment thereof, is a V_{H} wherein one or more amino acid residues have been substituted without substantially altering the antigen binding capacity.

Another embodiment of the present invention is a polypeptide as defined above wherein at least one heavy chain antibody, or a functional fragment thereof, is a V_{H} in which one or more amino acid residues have been substituted by specific nanobody sequences or amino acid residues.

Another embodiment of the present invention is a polypeptide as defined above, wherein at least one heavy chain antibody, or a functional fragment thereof, is humanized.

Another embodiment of the present invention is a polypeptide as defined above, wherein at least one heavy chain antibody, or a functional fragment thereof, comprises a human framework sequence.

Another embodiment of the present invention is a polypeptide as defined above, wherein said human framework sequence comprises amino acid sequences corresponding to framework regions encoded by human germline antibody gene segments.

Another embodiment of the present invention is a polypeptide as defined above wherein said human framework sequence is comprised in any of the framework regions of any of DP-29, DP-47 and DP-51.

Another embodiment of the present invention is a polypeptide as defined above, wherein said human framework sequence is one or more of FR1, FR2 or FR3, the remaining framework regions being selected from the equivalent FR1, FR2 and FR3 frameworks of the heavy chain antibody.

Another embodiment of the present invention is a nucleic acid capable of encoding a polypeptide as defined above.

Another embodiment of the present invention is a composition comprising a polypeptide and/or nucleic as defined above.

Another embodiment of the present invention is a composition comprising a polypeptide and/or nucleic as defined above and at least one anti-tangle agent, for simultaneous, separate or sequential administration to a subject.

Another embodiment of the present invention is a composition as defined above wherein said anti-tangle agent is covalently or non-covalently associated to said polypeptide.

Another embodiment of the present invention is a composition as defined above further comprising a pharmaceutically acceptable vehicle.

Another embodiment of the present invention is as defined above, or a nucleic acid as defined above, or a composition as defined above for use as a medicament.

Another embodiment of the present invention is a polypeptide as defined above, or a nucleic acid as defined above, or a composition as defined above for use in the treatment, prevention and/or alleviation of disorders mediated by amyloid plaque formation.

Another embodiment of the present invention is a use of a polypeptide as defined above, or a nucleic acid as defined above, or a composition as defined above for the preparation of a medicament for the treatment, prevention and/or alleviation of disorders mediated by amyloid plaque formation.

Another embodiment of the present invention is a polypeptide, nucleic acid or composition or use thereof as defined above wherein said disorder is Alzheimer's disease.

Another embodiment of the present invention is a polypeptide, nucleic acid or composition as defined above or a use of a polypeptide as defined above wherein said polypeptide is administered intravenously, subcutaneously, orally, sublingually, nasally or by inhalation.

Another embodiment of the present invention is a method of prophylactically or therapeutically treating Alzheimer's disease, comprising administering to the patient an effective dosage of a composition as defined above.

Another embodiment of the present invention is a method of producing a polypeptide as defined above comprising:
a) culturing host cells comprising nucleic acid capable of encoding a polypeptide as defined above under conditions allowing the expression of the polypeptide, and,
b) recovering the produced polypeptide from the culture.

Another embodiment of the present invention is a method as defined above, wherein said host cells are bacterial or yeast. Another embodiment of the present invention is a method of diagnosing a disease or disorder mediated by amyloid plaque formation comprising the steps of:
a) contacting a sample with a polypeptide as defined above, and
b) detecting binding of said polypeptide to said sample, and
c) comparing the binding detected in step (b) with a standard, wherein a difference in binding relative to said sample is diagnostic of a disease or disorder characterised by amyloid plaque formation.

Another embodiment of the present invention is a method of diagnosing a disease or disorder mediated by amyloid plaque formation comprising the steps of:
a) contacting a sample with a polypeptide as defined above, and
b) determining the amount of A-beta in the sample
c) comparing the amount determined in step (b) with a standard, wherein a difference in amount relative to said sample is diagnostic of a disease or disorder characterised by amyloid plaque formation.

Another embodiment of the present invention is a kit for diagnosing a disease or disorder mediated by amyloid plaque formation for use in a method as defined above.

Another embodiment of the present invention is a kit for diagnosing a disease or disorder mediated by amyloid plaque formation comprising a polypeptide as defined above.

Another embodiment of the present invention is a polypeptide as defined above further comprising one or more in vivo imaging agents.

The present invention relates to an anti-A-beta polypeptide comprising one or more Nanobodies directed against amyloid-beta (A-beta) or fragment thereof. The inventors have found that such polypeptide has an effect on the clearance of amyloid plaques and/or neurofibrillary tangles in the brain of neurodegenerative disease patients, e.g. AD subjects.

The present inventors clearly show that the anti-A-beta polypeptides of the present invention have a beneficial effect in APP transgenic mice.

A-beta related diseases for which the polypeptides of the present invention may have an effect are degenerative neural diseases related to invasive neural depositions.

One embodiment of the present invention relates to a polypeptide comprising at least one Nanobody capable of clearance of amyloid plaque from the brain or other parts in the body.

Another embodiment of the present invention relates to a polypeptide comprising at least one Nanobody capable of inhibiting the interaction between A-beta and another A-beta or fragments of A-beta.

According to one aspect of the invention, a polypeptide of the invention may be used to treat or alleviate the symptoms of degenerative neural diseases related to invasive neural depositions.

According to one aspect of the invention, a polypeptide of the invention may be used to prevent degenerative neural diseases related to invasive neural depositions i.e. prophylactic use. Such use is applicable in cases where patients have high risk to, for example, the early-onset familial AD.

These neural and other related non-neural diseases include, but are not limited to Adult Down Syndrome, Alzheimer's Disease, Amyotrophic Lateral Sclerosis/Parkinsonism Dementia Complex, Amyloid Polyneuropathy, Amyloid Cardiomyopathy, Amyloid in dialysis patients, Beta2-Microglobulin, Beta2-Amyloid deposits in muscle wasting disease, Corticobasal Degeneration, Creutzfeldt-Jacob Disease, Dementia Pugilistica, Fatal Familial Insomnia, Gerstamnn-Straussler-Scheinker Syndrome, Guam-Parkinsonism dementia complex, Hallervorden-Spatz Disease, Hereditary Cerebral Hemorrhage with Amyloidosis, Idiopathetic Myeloma, Inclusion Body Myositis, Islets of Langerhans Diabetes Type2 Insulinoma, Kura, Medullary Carcinoma of the Thyroid, Mediterranean Fever, Muckle-Wells Syndrome, Neurovisceral Lipid Storage Disease, Parkinson's Disease, Pick's Disease, Polyglutamine diseases including Huntington's Disease, Kennedy's Disease and all forms of Spinocerebellar Ataxia involving extended polyglutamine tracts, Progressive Supranuclear Palsy, Subacute Sclerosing Panencephalitis, Systemic Senile Amyloidosis, Scrapie.

One embodiment of the present invention relates to a pharmaceutical composition comprising at least one polypeptide of the invention and at least a pharmaceutical acceptable carrier, diluent or excipiens.

According to one preferred, but non-limiting embodiment, said pharmaceutical composition is suitable for oral administration.

The anti-A-beta polypeptides of the present invention bind to A-beta. According to one aspect of the invention, the anti-A-beta polypeptide binds to a target A-beta, and inhibits its interaction with one or more other A-betas. The target A-beta may be as part of a plaque, in suspension or solution or one or more of these. The other A-betas may also be as part of a plaque, in suspension or solution or one or more of these.

An ELISA assay to measure the binding of an anti-A-beta polypeptide is well known.

An assay to measure the extent of inhibitory action of anti-A-beta polypeptide is for example a depolymerization assay to measure the release of biotinylated A-beta from aggregated A-beta.

According to one aspect of the invention, an anti-A-beta polypeptide exhibits inhibitory action when its presence reduces the binding between A-beta and another A-beta, compared with A-beta-A-beta binding in the absence of a polypeptide. According to one aspect of the invention, the binding in the presence of an anti-beta polypeptide is reduced by more than 1, 2, 3, 4, 5, 6, 7, 8, 9, 10, 15, 20, 25, 30, 35, 40, 45, 50, 55, 60, 65, 70 or 75% compared with the binding in the absence of said polypeptide.

According to one aspect of the invention, Nanobodies are derived from heavy chain antibodies whose framework regions and complementary determining regions are part of a single domain polypeptide. Examples of such heavy chain antibodies include, but are not limited to, naturally occurring immunoglobulins devoid of light chains. Such immunoglobulins are disclosed in WO 94/04678 for example.

The antigen-binding site of this unusual class of heavy chain antibodies has a unique structure that comprises a single variable domain. For clarity reasons, the variable domain derived from a heavy chain antibody naturally devoid of light chain is known herein as a V_{HH} or V_{HH} domain or nanobody. Such a V_{HH} domain peptide can be derived from antibodies raised in Camelidae species, for example in camel, dromedary, llama, alpaca and guanaco.

Other species besides Camelidae (e.g. shark, pufferfish) may produce functional antigen-binding heavy chain antibodies naturally devoid of light chain. Such V_{HH} domains are within the scope of the invention.

Camelidae antibodies express a unique, extensive repertoire of functional heavy chain antibodies that lack light chains. The V_{HH} molecules derived from Camelidae antibodies are the smallest intact antigen-binding domains known (approximately 15 kDa, or 10 times smaller than conventional IgG) and hence are well suited towards delivery to dense tissues and for accessing the limited space between macromolecules.

Other examples of heavy chain antibodies include heavy chain antibodies derived from conventional four chain antibodies which have been modified by substituting one or more amino acid residues with Camelidae-specific residues (so-called camelisation, WO 94/04678). Such positions may preferentially occur at the V_{H}-V_{L} interface and at the so-called Camelidae hallmark residues (WO 94/04678), comprising positions 37, 44, 45, 47, 103 and 108.

The V_{HH} fragments of such heavy chain antibodies correspond to small, robust and efficient recognition units formed by a single immunoglobulin (Ig) domain.

The anti-A-beta polypeptides as disclosed herein and their derivatives not only possess the advantageous characteristics of conventional antibodies, such as low toxicity and high selectivity, but they also exhibit additional properties. They are more soluble; as such they may be stored and/or administered in higher concentrations compared with conventional antibodies.

Conventional antibodies are not stable at room temperature, and have to be refrigerated for preparation and storage, requiring necessary refrigerated laboratory equipment, storage and transport, which contribute towards time and expense. The anti-A-beta polypeptides of the present invention are stable at room temperature; as such they may be prepared, stored and/or transported without the use of refrigeration equipment, conveying a cost, time and environmental savings. Furthermore, conventional antibodies are unsuitable for use in assays or kits performed at temperatures outside biologically active-temperature ranges (e.g. 37±20° C.).

Other advantageous characteristics of the anti-A-beta polypeptides as disclosed herein as compared to conventional antibodies include modulation of half-life in the circulation which may be modulated according to the invention by, for example, albumin-coupling, or by coupling to one or more Nanobodies directed against a serum protein such as, for example, serum albumin. One aspect of the invention is a bispecific anti-A-beta polypeptide, with one specificity against a serum protein such as serum albumin and the other against the target as disclosed in WO04/041865 and incorporated herein by reference. Other means to enhance half life include coupling a polypeptide of the present invention to Fc, or to other Nanobodies directed against A-beta (i.e. creating multivalent Nanobodies—bivalent, trivalent, etc.) or coupling to polyethylene glycol. A controllable half-life is desirable for modulating dosage with immediate effect.

Conventional antibodies are unsuitable for use in environments outside the usual physiological pH range. They are unstable at low or high pH and hence are not suitable for oral administration. Camelidae antibodies resist harsh conditions, such as extreme pH, denaturing reagents and high temperatures, so making the anti-A-beta polypeptides as disclosed herein suitable for delivery by oral administration. Camelidae antibodies are resistant to the action of proteases which is less the case for conventional antibodies.

The yields of expression of conventional antibodies are very low and the method of production is very labor intensive. Furthermore, the manufacture or small-scale production of said antibodies is expensive because the mammalian cellular systems necessary for the expression of intact and active antibodies require high levels of support in terms of time and equipment, and yields are very low. The anti-A-beta polypeptides of the present invention may be cost-effectively produced through fermentation in convenient recombinant host organisms such as Escherichia coli and yeast; unlike conventional antibodies which also require expensive mammalian cell culture facilities, achievable levels of expression are high. Examples of yields of the polypeptides of the present invention are 1 to 10 mg/ml (E. coli) and up to 1 g/l (yeast).

The anti-A-beta polypeptides of the present invention exhibit high binding affinity for a broad range of different antigen types, and ability to bind to epitopes not recognised by conventional antibodies; for example they display long CDR3 loops with the potential to penetrate into cavities.

The anti-A-beta polypeptides of the present invention exhibit a straightforward generation of bi- or multi-functional molecules by (head-to-tail) fusion as disclosed in WO96/34103 (incorporated herein by reference).

Through their small size, the anti-A-beta polypeptides of the present invention allow better tissue penetration and ability to reach all parts of the body than conventional antibodies.

Llama single-domain antibodies can transmigrate across human blood-brain barrier. In one embodiment of the invention the anti-A-beta polypeptides can penetrate the blood-brain-barrier. In another embodiment of the invention the anti-A-beta polypeptides may not penetrate the blood-brain barrier.

The anti-A-beta polypeptides as disclosed herein are less immunogenic than conventional antibodies. A subclass of Camelidae antibodies has been discovered which displays 95% amino acid sequence homology to human V_{H} framework regions. This suggests that immunogenicity upon administration in human patients can be anticipated to be minor or even non-existent. Alternatively, if so required, humanization of nanobodies surprisingly requires only a few residues that need to be substituted.

One aspect of the invention is an anti-A-beta polypeptide comprising at least one anti-A-beta heavy chain antibody, and in particular a Nanobody derived therefrom. It is an aspect of the invention that such a polypeptide may comprise additional components. Such components may be polypeptide sequences, for example, one or more anti-A-beta Nanobodies, one or more anti-serum albumin Nanobodies, one more more anti-tau Nanobodies. Other fusion proteins are within the scope of the invention, and may include, for example, fusions with carrier polypeptides, signaling molecules, tags, and enzymes. Other components may include, for example, radiolabels, organic dyes, fluorescent compounds, Examples of an anti-A-beta polypeptide of the invention comprising one anti-A-beta nanobody are the polypeptides corresponding to a sequence represented by any of SEQ ID NOs: 117-183.

According to one preferred, but non-limiting embodiment, a polypeptide of the invention has an iso-electrical point between 4 and 11.

Preferably, a polypeptide of the invention has an iso-electrical point between 5 and 10.

According to one preferred, but non-limiting embodiment, the polypeptides of the invention comprise two amino acid chains (herein called “heavy chains”) which are covalently linked.

The heavy chains of the invention are preferably linked via a disulfide bond.

More preferably, the heavy chains of the invention are linked via cysteine residues forming a disulfide bond.

According to one preferred, but non-limiting embodiment, the heavy chains of the invention have an approximate molecular weight of between 35 kdal and 50 kdal. The molecular weight is determined as described in Hamers-Casterman et al. (Nature 1993).

Preferably, the heavy chains of the invention have a molecular weight of between 40 kdal and 50 kdal.

More preferably, the heavy chains of the invention have a molecular weight of between 41 kdal and 49 kdal, 42 kdal and 48 kdal, 43 kdal and 47 kdal, or 44 kdal and 46 kdal.

Most preferably, the heavy chains of the invention have a molecular weight of between 43 kdal and 46 kdal.

According to one preferred, but non-limiting embodiment, the heavy chains of the invention have a molecular weight of 43 kdal.

According to another preferred, but non-limiting embodiment, the heavy chains of the invention have a molecular weight of 46 kdal.

According to another aspect of the invention, an anti-A-beta polypeptide may comprise at least two anti-A-beta Nanobodies. It is an aspect of the invention that such a polypeptide may comprise additional components as described above.

According to a further aspect of the invention, an anti-A-beta polypeptide of the invention may comprise 1, 2, 3, 4, 5, 6, 7, 8, 9, 10, 11, 12, 13, 14, 15 or more than 15 Nanobodies directed against A-beta.

According to an aspect of the invention, an anti-A-beta polypeptide of the invention may comprise at least two identical or non identical anti-A-beta Nanobody sequences. It may be an aspect of the invention that at least two of the aforementioned sequences do not have equal affinity for A-beta, so forming an anti-A-beta polypeptide combining weak and high affinity binding sequences.

Methods of constructing bivalent polypeptides are known in the art (e.g. US 2003/0088074), and are also described below.

It may be desirable to modify the anti-A-beta polypeptide of the invention with respect to effector function so as to enhance its therapeutic efficacy. For example, nanobody-fusions with certain Fc domains may be advantageous, especially with Fc domains of human origin.

The present invention also relates to the finding that an anti-A-beta polypeptide as disclosed herein further comprising one or more Nanobodies each directed against a serum protein of a subject, surprisingly has significantly prolonged half-life in the circulation of said subject compared with the half-life of the anti-A-beta Nanobody(ies) when not part of said polypeptide. Furthermore, said anti-A-beta polypeptides were found to exhibit the same favourable properties of nanobodies as described above, such as, for example, high stability remaining intact in mice, extreme pH resistance, high temperature stability and high target specificity and affinity.

Thus, an anti-A-beta polypeptide as disclosed herein comprising one or more Nanobodies directed against A-beta and one or more Nanobodies with specificity to a serum protein is much more efficient than a polypeptide only targeting A-beta.

The serum protein may be any suitable protein found in the serum of a subject, or fragment thereof. In one aspect of the invention, the serum protein is any of serum albumin, serum immunoglobulins, thyroxine-binding protein, transferrin or fibrinogen. The subject may be, for example, rabbit, goat, mice, rat, cow, calve, camel, llama, monkey, donkey, guinea pig, chicken, sheep, dog, cat, horse, and preferably human. Depending on the intended use such as the required half-life for effective treatment and/or compartmentalization of the target antigen, the Nanobody partner can be directed to one of the above serum proteins.

According to one aspect of the invention, the number of Nanobodies directed against a serum protein in an anti-A-beta polypeptide of the invention is 1, 2, 3, 4, 5, 6, 7, 8, 9, 10, 11, 12, 13, 14, 15 or more than 15.

Another aspect of the invention is an anti-A-beta polypeptide further comprising at least one substance, covalently (joined) or non-covalently bound, directed to improving the half-life of the polypeptide in vivo. Examples of substances which improve the half-lives are known in the art and include, for example, polyethylene glycol and serum albumin.

Methods for joining Nanobodies and other substances to form bi and multi-specific polypeptides are known to the skilled person, and described below.

Polypeptides of the invention not modified according to the present invention to increase-half life, have the characteristic of rapid clearance from the body. Conversely, bispecific polypeptides comprising one or more Nanobodies directed against A-beta and one or more anti-serum protein Nanobodies are able to circulate in the subject's serum for several days, reducing the frequency of treatment, increasing the persistence times of the functional activity in the body, reducing the inconvenience to the subject and resulting in a decreased cost of treatment. The same advantageous characteristics are observable for polypeptides of the present invention comprising other substances aimed at improving the half life. Furthermore, it is an aspect of the invention that the half-life of the anti-A-beta polypeptides disclosed herein may be controlled by the number of anti-serum protein Nanobodies present in the polypeptide. A controllable half-life is desirable in several circumstances, for example, in the application of a timed dose of a therapeutic anti-A-beta polypeptide.

Methods for pharmacokinetic analysis and determination of half-life are familiar to those skilled in the art. Details may be found in Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2 nd Rev. ex edition (1982).

According to one aspect of the invention the polypeptides are capable of binding to one or more molecules which can increase the half-life of the polypeptide in vivo.

Half-life is the time taken for the serum concentration of the polypeptide to reduce by 50%, in vivo, for example due to degradation of the ligand and/or clearance or sequestration of the ligand by natural mechanisms. The polypeptides of the invention are stabilised in vivo and their half-life increased by binding to molecules which resist degradation and/or clearance or sequestration. Typically, such molecules are naturally occurring proteins which themselves have a long half-life in vivo.

The half-life of a polypeptide of the invention is increased if its functional activity persists, in vivo, for a longer period than a similar polypeptide which is not specific for the half-life increasing molecule. Thus, a polypeptide of the invention specific for HSA and a target molecule is compared with the same polypeptide wherein the specificity for HSA is not present, that it does not bind HSA but binds another molecule. For example, it may bind a second epitope on the target molecule. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible.

Typically, molecules which can increase the half-life of the polypeptide in vivo are polypeptides which occur naturally in vivo and which resist degradation or removal by endogenous mechanisms which remove unwanted material from the organism. For example, the molecule which increases the half-life of the organism may be selected from the following: (i) proteins from the extracellular matrix; for example collagen, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, invertebral disc, notochord, vitreous humour of the eye; (ii) proteins found in blood, including: plasma proteins such as fibrin, alpha-2 macroglobulin, serum albumin, fibrinogen A, fibrinogen B, serum amyloid protein A, heptaglobin, profilin, ubiquitin, uteroglobulin and beta-2-microglobulin; (iii) enzymes and inhibitors such as plasminogen, lysozyme, cystatin C, alpha-1-antitrypsin and pancreatic trypsin inhibitor. Plasminogen is the inactive precursor of the trypsin-like serine protease plasmin. It is normally found circulating through the blood stream. When plasminogen becomes activated and is converted to plasmin, it unfolds a potent enzymatic domain that dissolves the fibrinogen fibers that entangle the blood cells in a blood clot. This is called fibrinolysis; (iv) immune system proteins, such as IgE, IgG, IgM; (v) transport proteins such as retinol binding protein, alpha-1 microglobulin; defensins such as beta-defensin 1, Neutrophil defensins 1, 2 and 3; (vi) proteins found at the blood brain barrier or in neural tissues, such as melanocortin receptor, myelin, ascorbate transporter; (vii) transferrin receptor specific ligand-neuropharmaceutical agent fusion proteins (see U.S. Pat. No. 5,977,307); (viii) brain capillary endothelial cell receptor, transferrin, transferrin receptor, insulin, insulin like growth factor 1 (IGF 1) receptor, insulin-like growth factor 2 (IGF 2) receptor, insulin receptor; (ix) proteins localised to the kidney, such as polycystin, type IV collagen, organic anion transporter KI, Heymann's antigen; (x) proteins localised to the liver, for example alcohol dehydrogenase, G250; (xi) blood coagulation factor X, Alpha1 antitrypsin, HNF 1alpha; (xii)

Proteins localised to the lung, such as secretory component (binds IgA); (xiii) Proteins localised to the heart, for example HSP 27. This is associated with dilated cardiomyopathy; (xiv) proteins localised to the skin, for example keratin; (xv) bone specific proteins, such as bone morphogenic proteins (BMPs), which are a subset of the transforming growth factor beta superfamily that demonstrate osteogenic activity. Examples include BMP-2, -4, -5, -6, -7 (also referred to as osteogenic protein (OP-1) and -8 (OP-2)); (xvi) tumour specific proteins, including human trophoblast antigen, herceptin receptor, oestrogen receptor, cathepsins eg cathepsin B (found in liver and spleen); (xvii) disease-specific proteins, such as antigens expressed only on activated T-cells: including LAG-3 (lymphocyte activation gene), osteoprotegerin ligand (OPGL), OX40 (a member of the TNF receptor family, expressed on activated T cells and the only costimulatory T cell molecule known to be specifically up-regulated in human T cell leukaemia virus type-I (HTLV-I)-producing cells); Metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-a (TGF a), tumor necrosis factor-alpha (TNF-alpha), angiogenin, interleukin-3 (IL-3), interleukin-8 (IL-8), plateletderived endothelial growth factor (PD-ECGF), placental growth factor (P1GF), midkine platelet-derived growth factor-BB (PDGF), fractalkine; (xix) stress proteins (heat shock proteins); (xx) HSPs are normally found intracellularly. When they are found extracellularly, it is an indicator that a cell has died and spilled out its contents. This unprogrammed cell death (necrosis) only occurs when as a result of trauma, disease or injury and therefore in vivo, extracellular HSPs trigger a response from the immune system that will fight infection and disease. A dual specific which binds to extracellular HSP can be localised to a disease site; (xxi) proteins involved in Fc transport: Brambell receptor (also known as FcRB). This Fc receptor has two functions, both of which are potentially useful for delivery. The functions are: the transport of IgG from mother to child across the placenta, and the protection of IgG from degradation thereby prolonging its serum half life of IgG. It is thought that the receptor recycles IgG from endosome (see Holliger et al, Nat Biotechnol 1997 July; 15(7):632-6).

Polypeptides according to the invention may be designed to be specific for the above targets without requiring any increase in or increasing half life in vivo. For example, polypeptides according to the invention can be specific for targets selected from the foregoing which are tissue-specific, thereby enabling tissue-specific targeting of the polypeptide, irrespective of any increase in half-life, although this may result. Moreover, where the polypeptide targets kidney or liver, this may redirect the polypeptide to an alternative clearance pathway in vivo (for example, the polypeptide may be directed away from liver clearance to kidney clearance).

Another embodiment of the present invention is an anti-A-beta polypeptide as disclosed herein, further comprising one or more anti-tangle agents. Such anti-tangle agents may be covalently or non-covalently attached.

Another embodiment of the present invention is an anti-A-beta polypeptide as disclosed herein, further comprising one or more anti-tangle agents, said agent being an anti-tau Nanobody.

Examples of anti-tangle agents may comprise anti-tau, anti-phosphorylation and/or anti-caspase agents or antibodies or fragments thereof.

While the anti-A-beta Nanobody may remove the plaque and early-stage tangles, the anti-tangle agents may remove the advanced tangles.

Such an anti-A-beta/anti-tangle agents combination targets both plaques and fibrillar tangles, and leads to a synergetic action i.e. an increased therapeutic effect compared to separate treatment regimens. Such combined therapy may be particular effective in late stage AD.

One aspect of the invention is an anti-A-beta polypeptide as disclosed herein further comprising one or more Nanobodies directed against tau.

Another aspect of the invention is an anti-A-beta polypeptide comprising one or more Nanobodies directed against A-beta and one or more Nanobodies directed against tau. The Nanobodies can be joined with or without a linker.

According to one aspect of the invention, the number of Nanobodies directed against protein tau in an anti-A-beta polypeptide of the invention is 1, 2, 3, 4, 5, 6, 7, 8, 9, 10, 11, 12, 13, 14, 15 or more than 15. Depending on the progression or stage of the disease, more anti-tau Nanobodies can be added to remove advanced or late-stage tangles or to keep up a maintenance dosage to prevent reformation of tangles.

Another aspect of the invention is an anti-A-beta polypeptide comprising one or more Nanobodies directed against A-beta and one or more Nanobodies directed against tau further comprising one or more Nanobodies directed against a serum protein for extending the half-life.

A further aspect of the invention is a composition comprising at least one anti-A-beta polypeptide as disclosed herein and at least one anti-tangle agent, for simultaneous, separate or sequential administration to a subject.

Yet a further aspect of the invention is a method for treating AD comprising administering to an individual an effective amount of at least one anti-A-beta polypeptide of the invention and at least one anti-tangle agent, simultaneously, separately or sequentially.

By simultaneous administration means the anti-A-beta polypeptide and the anti-tangle agent are administered to a subject at the same time. For example, as a mixture of the polypeptide and agent, or a composition comprising said polypeptide and agent. Examples include, but are not limited to a solution administered intraveneously, a tablet, liquid, topical cream, etc., wherein each preparation comprises the polypeptide and agent of interest.

By separate administration means the anti-A-beta polypeptide and the anti-tangle agent are administered to a subject at the same time or substantially the same time. The polypeptide and agent are administered as separate, unmixed preparations. For example, the polypeptide and agent may be present in the kit as individual tablets. The tablets may be administered to the subject by swallowing both tablets at the same time, or one tablet directly following the other.

By sequential administration means the anti-A-beta polypeptide and the anti-tangle agent are administered to a subject sequentially. The polypeptide and agent are present in the kit as separate, unmixed preparations. There is a time interval between doses. For example, the polypeptide might be administered up to 336, 312, 288, 264, 240, 216, 192, 168, 144, 120, 96, 72, 48, 24, 20, 16, 12, 8, 4, 2, 1, or 0.5 hours after the agent, or vice versa.

In sequential administration, a polypeptide may be administered once, or any number of times and in various doses before and/or after administration of the agent. Sequential administration may be combined with simultaneous or sequential administration.

Another embodiment of the present invention is an anti-A-beta polypeptide as described herein in which one or more Nanobodies is humanized. The humanized Nanobody may be an anti-A-beta Nanobody, an anti-serum albumin, anti-protein tau, other Nanobody useful according to the invention, or a combination of these.

One embodiment of the invention, is an anti-A-beta polypeptide Nanobody comprising one or more humanized anti-A-beta Nanobodies and one or more humanized anti-human serum albumin Nanobodies.

By humanized is meant mutated so that potential immunogenicity upon administration in human patients is minor or nonexistent. Humanizing a polypeptide, according to the present invention, may comprise a step of replacing one or more of the non-human immunoglobulin amino acids by their human counterpart as found in a human consensus sequence or human germline gene sequence, without that polypeptide losing its typical character, i.e. the humanization does not significantly affect the antigen binding capacity of the resulting polypeptide.

According to one aspect of the invention, a humanized Nanobody is defined as a Nanobody having at least 50% homology (e.g. 55%, 60%, 65%, 70%, 75%, 80%, 85%, 90%, 95%, 98%, 100%) to the human framework region.

The inventors have determined the amino acid residues of a Nanobody which may be modified without diminishing the native affinity, in order to reduce its immunogenicity with respect to a heterologous species.

The inventors have also found that humanization of Nanobody polypeptides requires the introduction and mutagenesis of only a limited number of amino acids in a single polypeptide chain without dramatic loss of binding and/or inhibition activity. This is in contrast to humanization of scFv, Fab, (Fab)₂ and IgG, which requires the introduction of assembly of both chains.

The inventors have surprisingly found that Nanobodies of the invention comprising framework sequences highly homologous to human germline sequences such as DP29, DP47 and DP51 are highly effective. They occur naturally in some species such as those of the Camelidae. Such nanobodies are characterised in that they carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45. In addition, they may carry the human germline ‘J’ tryptophan at position 103, according to the Kabat numbering. The new class of nanobodies described in this invention is represented by SEQ ID NOs: 3, 4 and 5. Camelidae antibodies of this class, or other mutated Nanobodies which carry one or more framework sequences of this class are within the scope of the present invention.

As such, Nanobodies belonging to the class mentioned above, or Nanobodies carrying mutations of this class show a high amino acid sequence homology to human V_{H} framework regions and polypeptides of the invention comprising these might be administered to a human directly without expectation of an unwanted immune response therefrom, and without the burden of further humanization. The invention also relates to nucleic acids capable of encoding said polypeptides.

A humanization technique may be performed by a method comprising the replacement of any of the Nanobody residues with the corresponding framework 1, 2 and 3 (FR1, FR2 and FR3) residues of germline V_{H} genes (such as DP 47, DP 29 and DP 51) either alone or in combination.

According to one aspect of the present invention, humanization of nanobodies is performed by substituting in said nanobodies one or more of the amino acids at the positions described below, with the corresponding amino acids from the framework of germline V_{H} genes, the numbering in accordance with the Kabat numbering:

FR1 amino acid residues 1, 3, 5, 14 and 24,

FR2 amino acids residues 44, 45 and 49,

FR3 amino acid residues 74, 77, 78, 83 and 84

FR4 (derived from the germline J segments) amino acid positions 104 and 105.

According to an aspect of the invention, a framework region of the nanobody which is unsubstituted remains as the original nanobody framework.

According to one aspect of the invention, the residues of one or more of FR1, FR2 and FR3 are substituted according to the above scheme.

According to one aspect of the invention, at least 1, 2, 3 or all the residues of FR1 are substituted according to the above scheme.

According to one aspect of the invention, at least 1, 2, 3 or all the residues of FR2 are substituted according to the above scheme.

According to one aspect of the invention, at least 1, 2, 3, 4, 5, 6 or all the residues of FR3 are substituted according to the above scheme.

According to one aspect of the invention, at least 1, 2, or 3 all the residues of FR4 are substituted according to the above scheme.

In another embodiment of the invention, a humanized Nanobody is obtained by grafting all or part of the nanobody CDR regions onto the germline human V_{H} framework scaffold.

According to one aspect of the present invention, humanization of a Nanobody is performed by substituting one or more of CDR1, CDR2 and CDR3 of said Nanobody onto the germline human V_{H} framework scaffold. Examples of suitable framework scaffold include those of DP47, DP29 and DP51.

The Nanobodies of the invention are obtained according to the above mentioned humanization methods are part of the present invention.

Conventional four chain antibodies directed against A-beta or protein tau may be camelized, i.e. mutated such that the light chains are removed and one or more amino acid residues are substituted with Camelidae-specific residues (see for example, WO 94/04678 which is incorporated herein by reference) Such positions may preferentially occur at the VH-VL interface and at the so-called Camelidae hallmark residues, comprising positions 37, 44, 45, 47, 103 and 108. Such camelized antibodies are Nanobodies according to the invention. Polypeptides wherein at least one Nanobody is a VH wherein one or more amino acid residues have been partially substituted by specific sequences or amino acid residues of nanobodies are Nanobodies according to the invention.

The Nanobodies as described above may be joined to form any of the anti-A-beta polypeptides disclosed herein comprising more than one Nanobody using methods known in the art. For example, they may be fused by chemical cross-linking by reacting amino acid residues with an organic derivatising agent such as described by Blattler et al (Biochemistry 24, 1517-1524; EP294703). Alternatively, the Nanobodies may be fused genetically at the DNA level i.e. a polynucleotide formed which encodes the complete anti-A-beta polypeptide comprising one or more anti-A-beta Nanobodies and optionally one or more anti-serum protein Nanobodies, and optionally one or more anti-protein tau Nanobodies. A method for producing bivalent or multivalent nanobodies is disclosed in PCT patent application WO 96/34103.

According to another aspect of the invention, Nanobodies can be linked to each other either directly or via a linker sequence. Such constructs are difficult to produce with conventional antibodies where due to steric hindrance of the bulky subunits, functionality will be lost or greatly diminished. As seen with the Nanobodies of the invention functionality is increased considerably when they are joined together, compared to the monovalent anti-A-beta polypeptide.

According to one aspect of the present invention, the Nanobodies are linked to each other directly, without use of a linker. Contrary to joining bulky conventional antibodies where a linker sequence is needed to retain binding activity in the two subunits, polypeptides of the invention can be linked directly thereby avoiding potential problems of the linker sequence, such as antigenicity when administered to a human subject, or instability of the linker sequence leading to dissociation of the subunits.

According to another aspect of the present invention, the Nanobodies are linked to each other via a peptide linker sequence. Such a linker sequence may be a naturally occurring sequence or a non-naturally occurring sequence. The linker sequence is expected to be non-immunogenic in the subject to which the anti-A-beta polypeptide is administered. The linker sequence may provide sufficient flexibility to the multivalent anti-A-beta polypeptide, at the same time being resistant to proteolytic degradation. A non-limiting example of a linker sequence is one that can be derived from the hinge region of nanobodies as described in WO 96/34103. Another example is the linker sequence 3a (Ala-Ala-Ala).

Alternative linker sequences constructed by the inventors for fusion of bispecific and bivalent anti-A-beta polypeptides are listed in pending international application PCT/EP2004/004928. One linker sequence is the llama upper long hinge region. The other linkers are Gly/Ser linkers of different length. It is obvious to the person skilled in the art that said sequence linkers can be used to fuse any two monovalent sequences of this invention.

According to an aspect of the invention an anti-A-beta polypeptide may be a homologous sequence of a full-length anti-A-beta polypeptide. According to another aspect of the invention, an anti-A-beta polypeptide may be a functional portion of a full-length anti-A-beta polypeptide. According to another aspect of the invention, an anti-A-beta polypeptide may be a functional portion of a homologous sequence of a full-length anti-A-beta polypeptide. According to an aspect of the invention an anti-A-beta polypeptide may comprise a sequence of an anti-A-beta polypeptide.

According to an aspect of the invention a Nanobody used to form an anti-A-beta polypeptide may be a complete Nanobody (e.g. a nanobodies) or a homologous sequence thereof. According to another aspect of the invention, a Nanobody used to form an anti-A-beta polypeptide may be a functional portion of a complete Nanobody. According to another aspect of the invention, a Nanobody used to form an anti-A-beta polypeptide may be a homologous sequence of a complete Nanobody. According to another aspect of the invention, a Nanobody used to form an anti-A-beta polypeptide may be a functional portion of a homologous sequence of a complete Nanobody. As stated elsewhere, a heavy chain antibody may be a nanobody.

As used herein, a homologous sequence of the present invention may comprise additions, deletions or substitutions of one or more amino acids, which do not substantially alter the functional characteristics of the polypeptides of the invention. The number of amino acid deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

A homologous sequence according to the present invention may be an anti-A-beta polypeptide modified by the addition, deletion or substitution of amino acids, said modification not substantially altering the functional characteristics compared with the unmodified polypeptide.

A homologous sequence according to the present invention may be a sequence which exists in other Camelidae species such as, for example, camel, dromedary, llama, alpaca, guanaco etc.

Where homologous sequence indicates sequence identity, it means a sequence which presents a high sequence identity (more than 70%, 75%, 80%, 85%, 90%, 95% or 98% sequence identity) with the parent sequence and is preferably characterised by similar properties of the parent sequence, namely binding to the same target.

A homologous nucleotide sequence according to the present invention may refer to nucleotide sequences of more than 50, 100, 200, 300, 400, 500, 600, 800 or 1000 nucleotides able to hybridize to the reverse-complement of the nucleotide sequence capable of encoding the parent sequence, under stringent hybridisation conditions (such as the ones described by Sambrook et al., Molecular Cloning, Laboratory Manuel, Cold Spring, Harbor Laboratory press, New York).

As used herein, a functional portion refers to a sequence of a heavy chain antibody or Nanobody that is of sufficient size such that the interaction of interest is maintained with affinity of 1×10⁻⁶ M or better.

Alternatively, a functional portion comprises a partial deletion of the complete amino acid sequence and still maintains the binding site(s) and protein domain(s) necessary for the binding of and interaction with the target.

Alternatively a functional portion of a heavy chain antibody or Nanobody of the invention comprises a partial deletion of the complete amino acid sequence and still maintains the binding site(s) and protein domain(s) necessary for the binding of and interaction with the target.

Alternatively a functional portion of any of the sequences represented by SEQ ID NOs: 73-105 or 117-183 is a polypeptide which comprises a partial deletion of the complete amino acid sequence and which still maintains the binding site(s) and protein domain(s) necessary for the inhibition of binding of A-beta to another A-beta.

Alternatively a functional portion of any of the sequences represented by SEQ ID NOs: 73-105 or 117-183 is a polypeptide which comprises a partial deletion of the complete amino acid sequence and which still maintains the binding site(s) and protein domain(s) necessary for the binding of and interaction with A-beta.

Alternatively a functional portion comprises a partial deletion of the complete amino acid sequence of a polypeptide and which still maintains the binding site(s) and protein domain(s) necessary for the binding of and interaction with the antigen against which it was raised. It includes, but is not limited to nanobodies.

As used herein, a functional portion refers to less than 100% of the complete sequence (e.g., 99%, 90%, 80%, 70%, 60%, 50%, 40%, 30%, 20%, 10%, 5%, 1% etc.), but comprises 5 or more amino acids or 15 or more nucleotides.

A homologous sequence of the present invention may include an anti-A-beta polypeptide which has been humanized. A homologous sequence of the present invention may further include an anti-tau polypeptide which has been humanized. The humanization of antibodies of the new class of nanobodies would further reduce the possibility of unwanted immunological reaction in a human individual upon administration.

Yet other examples of heavy chain antibodies or Nanobodies include “functional fragments”, meaning fragments that are functional in antigen binding (as described in WO03/035694). Such fragments comprise active antigen binding regions. Such fragments may be fragments of functional heavy chain antibodies or Nanobodies as described above, fragments of molecules that behave like functional heavy chain antibodies or Nanobodies, fragments of functionalized antibodies, or fragments of heavy chain antibodies derived from conventional four chain antibodies which have been modified by substituting one or more amino acid residues with Camelidae-specific residues.

“Functional” in reference to a heavy chain antibody, a Nanobody, a V_{H} domain or fragments thereof means that the same retains a significant binding (dissociation constant in the micromolar range or better) to its epitope, compared with its binding in vivo, and that it shows no or limited aggregation (soluble and non-aggregated above 1 mg/ml), so allowing the use of the antibody as a binder.

“Functionalized” in reference to a heavy chain antibody, a Nanobody or fragments thereof means to render said heavy chain antibody, Nanobody or fragments thereof functional.

By “fragments thereof” as used in the sense of functional fragments, is meant a portion corresponding to more than 95% of the sequence, more than 90% of the sequence, more than 85% of the sequence, more than 80% of the sequence, more than 75% of the sequence, more than 70% of the sequence, more than 65% of the sequence, more than 60% of the sequence, more than 55% of the sequence, or more than 50% of the sequence.

According to the invention, a target is any of A-beta, tau or serum protein. Said targets are mammalian, and are derived from species such as rabbits, goats, mice, rats, cows, calves, camels, llamas, monkeys, donkeys, guinea pigs, chickens, sheep, dogs, cats, horses, and preferably humans.

Targets as mentioned herein such as A-beta, tau and serum proteins (e.g. serum albumin, serum immunoglobulins, thyroxine-binding protein, transferrin, fibrinogen) may be fragments of said targets. Thus a target is also a fragment of said target, capable of eliciting an immune response. A target is also a fragment of said target, capable of binding to a heavy chain antibody or Nanobody raised against the full length target.

A heavy chain antibody or Nanobody directed against a target means a heavy chain antibody or Nanobody that it is capable of binding to its target with an affinity of better than 10⁻⁶ M.

A-beta is to be understood as full-length A-beta or any fragment of A-beta. A-beta fragments are any A-beta created following a secretase mediated cleavage of APP and APLP or any other A-beta created directly or intermediately by any other process. Examples of A-beta fragments comprise but are not limited to the fragments obtained after cleavage as described in the background section above. Examples of fragments include A-beta (1-42) and A-beta (1-40).

A fragment as used herein refers to less than 100% of the sequence (e.g., 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is of sufficient length such that the interaction of interest is maintained with affinity of 1×10⁻⁶ M or better.

A fragment as used herein also refers to optional insertions, deletions and substitutions of one or more amino acids which do not substantially alter the ability of the target to bind to a Nanobody raised against the wild-type target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

One embodiment of the present invention relates to a polypeptide comprising at least one Nanobody wherein one or more amino acid residues have been substituted without substantially altering the antigen binding capacity.

Another embodiment of the present invention relates to a polypeptide comprising at least one Nanobody capable of binding to an A-beta neo-epitope created or exposed following a secretase mediated cleavage of APP and APLP or any other cleavage resulting in an A-beta cleavage product, such as, for example, cleavage by BACE1 or BACE2.

Targets as mentioned herein such as A-beta, tau and serum proteins may be a sequence which exists in any species including, but not limited to mouse, human, camel, llama, shark, pufferfish, goat, rabbit, bovine.

A target may be a homologous sequence of a complete target. A target may be a fragment of a homologous sequence of a complete target.

The skilled person will recognise that the anti-A-beta and anti-tau polypeptides of the present invention may be modified, and such modifications are within the scope of the invention. For example, the polypeptides may be used as drug carriers, in which case they may be fused to a therapeutically active agent, or they their solubility properties may be altered by fusion to ionic/bipolar groups, or they may be used in imaging by fusion to an appropriate imaging marker, or they may comprise modified amino acids etc. They may be also be prepared as salts. Such modifications which retain essentially the binding to A-beta and/or protein tau are within the scope of the invention.

As will be clear from the disclosure herein, it is also within the scope of the invention to use natural or synthetic analogs, mutants, variants, alleles, homologs and orthologs (herein collectively referred to as “analogs”) of the Nanobodies of the invention as defined herein, and in particular analogs of the Nanobodies of SEQ ID NO's 73-105. Thus, according to one embodiment of the invention, the term “Nanobody of the invention” in its broadest sense also covers such analogs.

Generally, in such analogs, one or more amino acid residues may have been replaced, deleted and/or added, compared to the Nanobodies of the invention as defined herein. Such substitutions, insertions or deletions may be made in one or more of the framework regions and/or in one or more of the CDR's. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

By means of non-limiting examples, a substitution may for example be a conservative substitution (as described herein) and/or an amino acid residue may be replaced by another amino acid residue that naturally occurs at the same position in another V_{HH} domain (see Tables 4-7 for some non-limiting examples of such substitutions), although the invention is generally not limited thereto. Thus, any one or more substitutions, deletions or insertions, or any combination thereof, that either improve the properties of the Nanobody of the invention or that at least do not detract too much from the desired properties or from the balance or combination of desired properties of the Nanobody of the invention (i.e. to the extent that the Nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the Nanobodies thus obtained.

For example, and depending on the host organism used to express the Nanobody or polypeptide of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation (again as described herein).

As can be seen from the data on the V_{HH} entropy and V_{HH} variability given in Tables 4 to 7 above, some amino acid residues in the framework regions are more conserved than others. Generally, although the invention in its broadest sense is not limited thereto, any substitutions, deletions or insertions are preferably made at positions that are less conserved. Also, generally, amino acid substitutions are preferred over amino acid deletions or insertions.

The analogs are preferably such that they can bind to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the analog against A-beta can be determined in a manner known per se, for example using the assay described herein.

The analogs are preferably also such that they retain the favourable properties the Nanobodies, as described herein.

Also, according to one preferred embodiment, the analogs have a degree of sequence identity of at least 70%, preferably at least 80%, more preferably at least 90%, such as at least 95% or 99% or more; and/or preferably have at most 20, preferably at most 10, even more preferably at most 5, such as 4, 3, 2 or only 1 amino acid difference (as defined herein), with one of the Nanobodies of SEQ ID NOs 73-105.

Also, the framework sequences and CDR's of the analogs are preferably such that they are in accordance with the preferred embodiments defined herein. More generally, as described herein, the analogs will have (a) a Q at position 108; and/or (b) a charged amino acid or a cysteine residue at position 45 and preferably an E at position, and more preferably E at position 44 and R at position 45; and/or (c) P, R or S at position 103.

One preferred class of analogs of the Nanobodies of the invention comprise Nanobodies that have been humanized (i.e. compared to the sequence of a naturally occurring Nanobody of the invention). As mentioned in the background art cited herein, such humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from the Tables herein, from the possible humanizing substitutions mentioned in the background art cited herein, and/or from a comparison between the sequence of a Nanobody and the sequence of a naturally occurring human V_{H} domain.

The humanizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Some preferred, but non-limiting examples of humanized Nanobodies of the invention are given in SEQ ID NO's 85-105.

Generally, as a result of humanization, the Nanobodies of the invention may become more “human-like”, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The humanized and other analogs, and nucleic acid sequences encoding the same, can be provided in any manner known per se. For example, the analogs can be obtained by providing a nucleic acid that encodes a naturally occurring V_{HH} domain, changing the codons for the one or more amino acid residues that are to be substituted into the codons for the corresponding desired amino acid residues (e.g. by site-directed mutagenesis or by PCR using suitable mismatch primers), expressing the nucleic acid/nucleotide sequence thus obtained in a suitable host or expression system; and optionally isolating and/or purifying the analog thus obtained to provide said analog in essentially isolated form (e.g. as further described herein). This can generally be performed using methods and techniques known per se, which will be clear to the skilled person, for example from the handbooks and references cited herein, the background art cited herein and/or from the further description herein. Alternatively, a nucleic acid encoding the desired analog can be synthesized in a manner known per se (for example using an automated apparatus for synthesizing nucleic acid sequences with a predefined amino acid sequence) and can then be expressed as described herein. Yet another technique may involve combining one or more naturally occurring and/or synthetic nucleic acid sequences each encoding a part of the desired analog, and then expressing the combined nucleic acid sequence as described herein. Also, the analogs can be provided using chemical synthesis of the pertinent amino acid sequence using techniques for peptide synthesis known per se, such as those mentioned herein.

In this respect, it will be also be clear to the skilled person that the Nanobodies of the invention (including their analogs) can be designed and/or prepared starting from human V_{H} sequences (i.e. amino acid sequences or the corresponding nucleotide sequences), such as for example from human V_{H}3 sequences such as DP-47, DP-51 or DP-29, i.e. by introducing one or more camelizing substitutions (i.e. changing one or more amino acid residues in the amino acid sequence of said human V_{H} domain into the amino acid residues that occur at the corresponding position in a V_{HH} domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human V_{H} domain as a starting point.

Some preferred, but non-limiting camelizing substitutions can be derived from Tables 4 to 7. It will also be clear that camelizing substitutions are one or more of the Hallmark residues will generally have a greater influence on the desired properties than substitutions at one or more of the other amino acid positions, although both and any suitable combination thereof are included within the scope of the invention. For example, it is possible to introduce one or more camelizing substitutions that already confer at least some the desired properties, and then to introduce further camelizing substitutions that either further improve said properties and/or confer additional favourable properties. Again, the skilled person will generally be able to determine and select suitable camelizing substitutions or suitable combinations of camelizing substitutions, based on the disclosure herein and optionally after a limited degree of routine experimentation, which may for example involve introducing a limited number of possible camelizing substitutions and determining whether the favourable properties of Nanobodies are obtained or improved (i.e. compared to the original V_{H} domain).

Generally, however, such camelizing substitutions are preferably such that the resulting an amino acid sequence at least contains (a) a Q at position 108; and/or (b) a charged amino acid or a cysteine residue at position 45 and preferably also an E at position, and more preferably E at position 44 and R at position 45; and/or (c) P, R or S at position 103; and optionally one or more further camelizing substitutions. More preferably, the camelizing substitutions are such that they result in a Nanobody of the invention and/or in an analog thereof (as defined herein), such as in a humanized analog and/or preferably in an analog that is as defined in the preceding paragraphs.

As will also be clear from the disclosure herein, it is also within the scope of the invention to use parts or fragments, or combinations of two or more parts or fragments, of the Nanobodies of the invention as defined herein, and in particular parts or fragments of the Nanobodies of SEQ ID NO's 73-105. Thus, according to one embodiment of the invention, the term “Nanobody of the invention” in its broadest sense also covers such parts or fragments.

Generally, such parts or fragments of the Nanobodies of the invention (including analogs thereof) have amino acid sequences in which, compared to the amino acid sequence of the corresponding full length Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

The parts or fragments are preferably such that they can bind to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the analog against A-beta can be determined in a manner known per se, for example using the assay described herein.

Any part or fragment is preferably such that it comprises at least 10 contiguous amino acid residues, preferably at least 20 contiguous amino acid residues, more preferably at least contiguous amino acid residues, such as at least 40 contiguous amino acid residues, of the amino acid sequence of the corresponding full length Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR's (and preferably at least CDR3 or part thereof) and at least one other CDR (i.e. CDR1 or CDR2) or at least part thereof, preferably connected by suitable framework sequence(s) or at least part thereof. More preferably, any part or fragment is such that it comprises at least one of the CDR's (and preferably at least CDR3 or part thereof) and at least part of the two remaining CDR's, again preferably connected by suitable framework sequence(s) or at least part thereof.

According to another particularly preferred, but non-limiting embodiment, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different Nanobodies of the invention), i.e. to provide an analog (as defined herein) and/or to provide further parts or fragments (as defined herein) of a Nanobody of the invention. It is for example also possible to combine one or more parts or fragments of a Nanobody of the invention with one or more parts or fragments of a human V_{H} domain.

According to one preferred embodiment, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NOs 73-105.

The parts and fragments, and nucleic acid sequences encoding the same, can be provided and optionally combined in any manner known per se. For example, such parts or fragments can be obtained by inserting a stop codon in a nucleic acid that encodes a full-sized Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody of the invention or by synthesizing such a nucleic acid in a manner known per se. Parts or fragments may also be provided using techniques for peptide synthesis known per se.

The invention in its broadest sense also comprises derivatives of the Nanobodies of the invention. Such derivatives can generally be obtained by modification, and in particular by chemical and/or biological (e.g enzymatical) modification, of the Nanobodies of the Invention and/or of one or more of the amino acid residues that form the Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody of the invention, and in particular of one or more functional groups, residues or moieties that confer one or more desired properties or functionalities to the Nanobody of the invention. Example of such functional groups will be clear to the skilled person.

For example, such modification may comprise the introduction (e.g. by covalent binding or in any other suitable manner) of one or more functional groups that that increase the half-life, the solubility and/or the absorption of the Nanobody of the invention, that reduce the immunogenicity and/or the toxicity of the Nanobody of the invention, that eliminate or attenuate any undesirable side effects of the Nanobody of the invention, and/or that confer other advantageous properties to and/or reduce the undesired properties of the Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv's and single domain antibodies), for which reference is for example made to Remington's Pharmaceutical Sciences, 16th ed., Mack Publishing Co., Easton, Pa. (1980). Such functional groups may for example be linked directly (for example covalently) to a Nanobody of the invention, or optionally via a suitable linker or spacer, as will again be clear to the skilled person.

One of the most widely used techniques for increasing the half-life and/or the reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or MnPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cystine-residue (see for example Yang et al., Protein Engineering, 16, 10, 761-770 (2003). For example, for this purpose, PEG may be attached to a cystine residue that naturally occurs in a Nanobody of the invention, a Nanobody of the invention may be modified so as to suitably introduce one or more cystine residues for attachment of PEG, or an amino acid sequence comprising one or more cystine residues for attachment of PEG may be fused to the N- and/or C-terminus of a Nanobody of the invention, all using techniques of protein engineering known per se to the skilled person.

Preferably, for the Nanobodies and proteins of the invention, a PEG is used with a molecular weight of more than 5000, such as more than 10.000 and less than 200.000, such as less than 100.000; for example in the range of 20.000-80.000.

Another, usually less preferred modification comprises N-linked or O-linked glycosylation, usually as part of co-translational and/or post-translational modification, depending on the host cell used for expressing the Nanobody or polypeptide of the invention.

Yet another modification may comprise the introduction of one or more detectable labels or other signal-generating groups or moieties, depending on the intended use of the labelled Nanobody. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as 3H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metals chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga or other metals or metallic cations that are particularly suited for use in in vivo, in vitro or in situ diagnosis and imaging, such as (¹⁵⁷Gd, ⁵⁵M, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alcaline phosphatase, asparaginase, glucose oxidase, β-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled Nanobodies and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaininetetraacetic acid (EDTA).

Yet another modification may comprise the introduction of a functional group that is one part of a specific binding pair, such as the biotin-(strept)avidin binding pair. Such a functional group may be used to link the Nanobody of the invention to another protein, polypeptide or chemical compound that is bound to the other half of the binding pair, i.e. through formation of the binding pair. For example, a Nanobody of the invention may be conjugated to biotin, and linked to another protein, polypeptide, compound or carrier conjugated to avidin or streptavidin. For example, such a conjugated Nanobody may be used as a reporter, for example in a diagnostic system where a detectable signal-producing agent is conjugated to avidin or streptavidin. Such binding pairs may for example also be used to bind the Nanobody of the invention to a carrier, including carriers suitable for pharmaceutical purposes. One non-limiting example are the liposomal formulations described by Cao and Suresh, Journal of Drug Targetting, 8, 4, 257 (2000). Such binding pairs may also be used to link a therapeutically active agent to the Nanobody of the invention.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, the derivatives are such that they bind to A-beta with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of a derivative of a Nanobody of the invention against A-beta can be determined in a manner known per se, for example using the assay described herein.

As mentioned above, the invention also relates to proteins or polypeptides that essentially consist of or comprise at least one Nanobody of the invention. By “essentially consist of” is meant that the amino acid sequence of the polypeptide of the invention either is exactly the same as the amino acid sequence of a Nanobody of the invention or corresponds to the amino acid sequence of a Nanobody of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody.

Said amino acid residues may or may not change, alter or otherwise influence the (biological) properties of the Nanobody and may or may not add further functionality to the Nanobody. For example, such amino acid residues:
a) can comprise an N-terminal Met residue, for example as result of expression in a heterologous host cell or host organism.
b) may form a signal sequence or leader sequence that directs secretion of the Nanobody from a host cell upon synthesis. Suitable secretory leader peptides will be clear to the skilled person, and may be as further described herein. Usually, such a leader sequence will be linked to the N-terminus of the Nanobody, although the invention in its broadest sense is not limited thereto;
c) may form a sequence or signal that allows the Nanobody to be directed towards and/or to penetrate or enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person. Some non-limiting examples are the small peptide vectors (“Pep-trans vectors”) described in WO 03/026700 and in Temsamani et al., Expert Opin. Biol. Ther., 1, 773 (2001); Temsamani and Vidal, Drug Discov. Today, 9, 1012 (004) and Rousselle, J. Pharmacol. Exp. Ther., 296, 124-131 (2001), and the membrane translocator sequence described by Zhao et al., Apoptosis, 8, 631-637 (2003). C-terminal and N-terminal amino acid sequences for intracellular targeting of antibody fragments are for example described by Cardinale et al., Methods, 34, 171 (2004). Other suitable techniques for intracellular targeting involve the expression and/or use of so-called “intrabodies” comprising a Nanobody of the invention, as mentioned below;
d) may form a “tag”, for example an amino acid sequence or residue that allows or facilitates the purification of the Nanobody, for example using affinity techniques directed against said sequence or residue. Thereafter, said sequence or residue may be removed (e.g. by chemical or enzymatical cleavage) to provide the Nanobody sequence (for this purpose, the tag may optionally be linked to the Nanobody sequence via a cleavable linker sequence or contain a cleavable motif). Some preferred, but non-limiting examples of such residues are multiple histidine residues, glutatione residues and a myc-tag such as AAAEQKLISEEDLNGAA [SEQ ID NO:31];
e) may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies of the invention;

According to another embodiment, a polypeptide of the invention comprises a Nanobody of the invention, which is fused at its amino terminal end, at its carboxy terminal end, or both at its amino terminal end and at its carboxy terminal end to at least one further amino acid sequence, i.e. so as to provide a fusion protein comprising said Nanobody of the invention and the one or more further amino acid sequences. Such a fusion will also be referred to herein as a “Nanobody fusion”.

The one or more further amino acid sequence may be any suitable and/or desired amino acid sequences. The further amino acid sequences may or may not change, alter or otherwise influence the (biological) properties of the Nanobody, and may or may not add further functionality to the Nanobody or the polypeptide of the invention. Preferably, the further amino acid sequence is such that it confers one or more desired properties or functionalities to the Nanobody or the polypeptide of the invention.

Example of such amino acid sequences will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005),

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody of the invention per se. Some non-limiting examples of such amino acid sequences are serum proteins, such as human serum albumin (see for example WO 00/27435) or haptenic molecules (for example haptens that are recognized by circulating antibodies, see for example WO 98/22141).

The further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope). For example, the further amino acid sequence may provide a second binding site that is directed against a serum protein (such as, for example, human serum albumin or another serum protein such as IgG), so as to provide increased half-life in serum. Reference is for example made to EP 0 368 684, WO 91/01743, WO 01/45746 and WO 04/003019 (in which various serum proteins are mentioned), as well as to Harmsen et al., Vaccine, 23 (41); 4926-42.

According to another embodiment, the one or more further amino acid sequences may comprises one or more parts, fragments or domains of conventional 4-chain antibodies (and in particular human antibodies) and/or of heavy chain antibodies. For example, although usually less preferred, a Nanobody of the invention may be linked to a conventional (preferably human) V_{H} or V_{L} domain domain or to a natural or synthetic analog of a V_{H} or V_{L} domain, again optionally via a linker sequence (including but not limited to other (single) domain antibodies, such as the dAb's described by Ward et al.).

The at least one Nanobody may also be linked to one or more (preferably human) CH₁, CH₂ and/or CH₃ domains, optionally via a linker sequence. For instance, a Nanobody linked to a suitable CH₁ domain could for example be used—together with suitable light chains—to generate antibody fragments/structures analogous to conventional Fab fragments or F(ab′)2 fragments, but in which one or (in case of an F(ab′)2 fragment) one or both of the conventional V_{H} domains have been replaced by a Nanobody of the invention. Also, two Nanobodies could be linked to a CH3 domain (optionally via a linker) to provide a construct with increased half-life in vivo.

According to one specific embodiment of a polypeptide of the invention, one or more Nanobodies of the invention may linked to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more CH₂ and/or CH₃ domains of an antibody, such as from a heavy chain antibody (as described herein) and more preferably from a conventional human 4-chain antibody; and/or may form (part of) and Fc region, for example from IgG, from IgE or from another human Ig.

For example, WO 94/04678 describes heavy chain antibodies comprising a Camelid V_{HH} domain or a humanized derivative thereof (i.e. a Nanobody), in which the Camelidae CH₂ and/or CH₃ domain have been replaced by human CH₂ and CH₃ domains, so as to provide an immunoglobulin that consists of 2 heavy chains each comprising a Nanobody and human CH2 and CH3 domains (but no CH1 domain), which immunoglobulin has the effector function provided by the CH2 and CH3 domains and which immunoglobulin can function without the presence of any light chains. Other amino acid sequences that can be suitably linked to the Nanobodies of the invention so as to provide an effector function will be clear to the skilled person, and may be chosen on the basis of the desired effector function(s). Reference is for example made to WO 04/058820, WO 99/42077 and WO 05/017148, as well as the review by Holliger and Hudson, supra. Coupling of a Nanobody of the invention to an Fc portion may also lead to an increased half-life, compared to the corresponding Nanobody of the invention. For some applications, the use of an Fc portion and/or of constant domains (i.e. CH2 and/or CH3 domains) that confer increased half-life without any biologically significant effector function may also be suitable or even preferred. Other suitable constructs comprising one or more Nanobodies and one or more constant domains with increased half-life in vivo will be clear to the skilled person, and may for example comprise two Nanobodies linked to a CH3 domain, optionally via a linker sequence.

Generally, any fusion protein or derivatives with increased half-life will preferably have a molecular weight of more than 50 kD, the cut-off value for renal absorption.

The further amino acid sequences may also form a signal sequence or leader sequence that directs secretion of the Nanobody or the polypeptide of the invention from a host cell upon synthesis (for example to provide a pre-, pro- or prepro-form of the polypeptide of the invention, depending on the host cell used to express the polypeptide of the invention).

The further amino acid sequence may also form a sequence or signal that allows the Nanobody or polypeptide of the invention to be directed towards and/or to penetrate or enter into specific organs, tissues, cells, or parts or compartments of cells, and/or that allows the Nanobody or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, the “Peptrans” vectors mentioned above, the sequences described by Cardinale et al. and the amino acid sequences and antibody fragments known per se that can be used to express or produce the Nanobodies and polypeptides of the invention as so-called “intrabodies”, for example as described in WO 94/02610, WO 95/22618, U.S. Pat. No. 6,004,940, WO 03/014960, WO 99/07414; WO 05/01690; EP 1 512 696; and in Cattaneo, A. & Biocca, S. (1997) Intracellular Antibodies: Development and Applications. Landes and Springer-Verlag; and in Kontermann, Methods 34, (2004), 163-170, and the further references described therein.

According to one preferred, but non-limiting embodiment, said one or more further amino acid sequences comprise at least one further Nanobody, so as to provide a polypeptide of the invention that comprises at least two, such as three, four, five or more Nanobodies, in which said Nanobodies may optionally be linked via one or more linker sequences (as defined herein). Polypeptides of the invention that comprise two or more Nanobodies, of which at least one is a Nanobody of the invention, will also be referred to herein as “multivalent” polypeptides of the invention, and the Nanobodies present in such polypeptides will also be referred to herein as being in a “multivalent format”. For example a “bivalent” polypeptide of the invention comprises two Nanobodies, optionally linked via a linker sequence, whereas a “trivalent” polypeptide of the invention comprises three Nanobodies, optionally linked via two linker sequences; etc.; in which at least one of the Nanobodies present in the polypeptide, and up to all of the Nanobodies present in the polypeptide, is/are a Nanobody of the invention.

In a multivalent polypeptide of the invention, the two or more Nanobodies may be the same or different, and may be directed against the same antigen or antigenic determinant (for example against the same part(s) or epitope(s) or against different parts or epitopes) or may alternatively be directed against different antigens or antigenic determinants; or any suitable combination thereof. For example, a bivalent polypeptide of the invention may comprise (a) two identical Nanobodies; (b) a first Nanobody directed against a first antigenic determinant of a protein or antigen and a second Nanobody directed against the same antigenic determinant of said protein or antigen which is different from the first Nanobody; (c) a first Nanobody directed against a first antigenic determinant of a protein or antigen and a second Nanobody directed against another antigenic determinant of said protein or antigen; or (d) a first Nanobody directed against a first protein or antigen and a second Nanobody directed against a second protein or antigen (i.e. different from said first antigen). Similarly, a trivalent polypeptide of the invention may, for example and without being limited thereto. comprise (a) three identical Nanobodies; (b) two identical Nanobody against a first antigenic determinant of an antigen and a third Nanobody directed against a different antigenic determinant of the same antigen; (c) two identical Nanobody against a first antigenic determinant of an antigen and a third Nanobody directed against a second antigen different from said first antigen; (d) a first Nanobody directed against a first antigenic determinant of a first antigen, a second Nanobody directed against a second antigenic determinant of said first antigen and a third Nanobody directed against a second antigen different from said first antigen; or (e) a first Nanobody directed against a first antigen, a second Nanobody directed against a second antigen different from said first antigen, and a third Nanobody directed against a third antigen different from said first and second antigen.

Polypeptides of the invention that contain at least two Nanobodies, in which at least one Nanobody is directed against a first antigen (i.e. against A-beta) and at least one Nanobody is directed against a second antigen (i.e. different from A-beta), will also be referred to as “multispecific” polypeptides of the invention, and the Nanobodies present in such polypeptides will also be referred to herein as being in a “multivalent format”. Thus, for example, a “bispecific” polypeptide of the invention is a polypeptide that comprises at least one Nanobody directed against a first antigen (i.e. A-beta) and at least one further Nanobody directed against a second antigen (i.e. different from A-beta), whereas a “trispecific” polypeptide of the invention is a polypeptide that comprises at least one Nanobody directed against a first antigen (i.e. A-beta), at least one further Nanobody directed against a second antigen (i.e. different from A-beta) and at least one further Nanobody directed against a third antigen (i.e. different from both A-beta and the second antigen); etc.

Accordingly, in its simplest form, a bispecific polypeptide of the invention is a bivalent polypeptide of the invention (as defined herein), comprising a first Nanobody directed against A-beta and a second Nanobody directed against a second antigen, in which said first and second Nanobody may optionally be linked via a linker sequence (as defined herein); whereas a trispecific polypeptide of the invention in its simplest form is a trivalent polypeptide of the invention (as defined herein), comprising a first Nanobody directed against A-beta, a second Nanobody directed against a second antigen and a third Nanobody directed against a third antigen, in which said first, second and third Nanobody may optionally be linked via one or more, and in particular one and more in particular two, linker sequences.

However, as will be clear from the description hereinabove, the invention is not limited thereto, in the sense that a multispecific polypeptide of the invention may comprise at least one Nanobody against A-beta and any number of Nanobodies directed against one or more antigens different from A-beta.

Furthermore, although it is encompassed within the scope of the invention that the specific order or arrangement of the various Nanobodies in the polypeptides of the invention may have some influence on the properties of the final polypeptide of the invention (including but not limited to the affinity, specificity or avidity for A-beta or against the one or more other antigens), said order or arrangement is usually not critical and may be suitably chosen by the skilled person, optionally after on some limited routine experiments based on the disclosure herein. Thus, when reference is made to a specific multivalent or multispecific polypeptide of the invention, it should be noted that this encompasses any order or arrangements of the relevant Nanobodies, unless explicitly indicated otherwise.

Finally, it is also within the scope of the invention that the polypeptides of the invention contain two or more Nanobodies and one or more further amino acid sequences (as mentioned herein).

For multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221. Some other examples of some specific multispecific and/or multivalent polypeptidee of the invention can be found in the applications by applicant referred to herein.

One preferred, but non-limiting example of a multispecific polypeptide of the invention comprises at least one Nanobody of the invention and at least one Nanobody that provides for an increased half-life. Some preferred, but non-limiting examples of such Nanobodies include Nanobodies directed against serum proteins, such as human serum albumin, thyroxine-binding protein, (human) transferrine, fibrinogen, an immunoglobulin such as IgG, IgE or IgM, or one of the other serum proteins listed herein or in WO 04/003019.

Preferably, said Nanobody that provides for an increased half-life is preferably a Nanobody that is directed against serum albumin, and in particular against a mammalian serum albumin. Usually, for pharmaceutical use, Nanobodies against human serum albumin will be preferred; however, for example, experiments in mice, rats, pigs or dogs, Nanobodies against mouse serum albumin (MSA), rats serum albumin, pig serum albumin or dog serum albumin, respectively, can be used. It is also possible to use Nanobodies directed against serum albumin from several different mammalian species Another embodiment of the present invention is a polypeptide construct as described above wherein said at least one (human) serum protein is any of (human) serum albumin, (human) serum immunoglobulins, (human)

According to a specific, but non-limiting aspect of the invention, the polypeptides of the invention contain, besides the one or more Nanobodies of the invention, at least one Nanobody against human serum albumin. Although these Nanobodies against human serum albumin may be as generally described in the applications by applicant cited above (see for example WO4/062551), according to a particularly preferred, but non-limiting embodiment, said Nanobody against human serum albumin consists of 4 framework regions (FR1 to FR4 respectively) and 3 complementarity determining regions (CDR1 to CDR3 respectively), in which:

i) CDR1 is an amino acid sequence chosen from the group consisting of:

| | |
|---|---|
| SFGMS | [SEQ ID NO: 15] |
| LNLMG | [SEQ ID NO: 16] |
| INLLG | [SEQ ID NO: 17] |
| NYWMY | [SEQ ID NO: 18] |

and/or from the group consisting of amino acid sequences that have 2 or only 1 “amino acid difference(s)” (as defined herein) with one of the above amino acid sequences, in which:

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences;

and in which:

ii) CDR2 is an amino acid sequence chosen from the group consisting of:

| | |
|---|---|
| SISGSGSDTLYADSVKG | [SEQ ID NO: 19] |
| TITVGDSTNYADSVKG | [SEQ ID NO: 20] |
| TITVGDSTSYADSVKG | [SEQ ID NO: 21] |
| SINGRGDDTRYADSVKG | [SEQ ID NO: 22] |
| AISADSSTKNYADSVKG | [SEQ ID NO: 23] |
| AISADSSDKRYADSVKG | [SEQ ID NO: 24] |
| RISTGGGYSYYADSVKG | [SEQ ID NO: 25] |

or from the group consisting of amino acid sequences that have at least 80%, preferably at least 90%, more preferably at least 95%, even more preferably at least 99% sequence identity (as defined herein) with one of the above amino acid sequences; in which

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences;

and/or from the group consisting of amino acid sequences that have 3, 2 or only 1 “amino acid difference(s)” (as defined herein) with one of the above amino acid sequences, in which:

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences;

and in which:

iii) CDR3 is an amino acid sequence chosen from the group consisting of:

| | |
|---|---|
| DREAQVDTLDFDY | [SEQ ID NO: 26] |

or from the group consisting of amino acid sequences that have at least 80%, preferably at least 90%, more preferably at least 95%, even more preferably at least 99% sequence identity (as defined herein) with one of the above amino acid sequences; in which

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences;

and/or from the group consisting of amino acid sequences that have 3, 2 or only 1 “amino acid difference(s)” (as defined herein) with one of the above amino acid sequences, in which:

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences;

or from the group consisting of:

| | |
|---|---|
| GGSLSR | [SEQ ID NO: 27] |
| RRTWHSEL | [SEQ ID NO: 28] |
| GRSVSRS | [SEQ ID NO: 29] |
| GRGSP | [SEQ ID NO: 30] |

and/or from the group consisting of amino acid sequences that have 3, 2 or only 1 “amino acid difference(s)” (as defined herein) with one of the above amino acid sequences, in which:

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences.

In another aspect, the invention relates to a Nanobody against human serum albumin, which consist of 4 framework regions (FR1 to FR4 respectively) and 3 complementarity determining regions (CDR1 to CDR3 respectively), which is chosen from the group consisting of Nanobodies with the one of the following combinations of CDR1, CDR2 and CDR3, respectively:

CDR1: SFGMS; CDR2: SISGSGSDTLYADSVKG; CDR3: GGSLSR;

CDR1: LNLMG; CDR2: TITVGDSTNYADSVKG; CDR3: RRTWHSEL;

CDR1: INLLG; CDR2: TITVGDSTSYADSVKG; CDR3: RRTWHSEL;

CDR1: SFGMS; CDR2: SINGRGDDTRYADSVKG; CDR3: GRSVSRS;

CDR1: SFGMS; CDR2: AISADSSDKRYADSVKG; CDR3: GRGSP;

CDR1: SFGMS; CDR2: AISADSSDKRYADSVKG; CDR3: GRGSP;

CDR1: NYWMY; CDR2: RISTGGGYSYYADSVKG; CDR3: DREAQVDTLDFDY.

In the Nanobodies of the invention that comprise the combinations of CDR's mentioned above, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR's; in which

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences;

and/or chosen from the group consisting of amino acid sequences that have 3, 2 or only 1 (as indicated in the preceding paragraph) “amino acid difference(s)” (as defined herein) with the mentioned CDR(s) one of the above amino acid sequences, in which:

(1) any amino acid substitution is preferably a conservative amino acid substitution (as defined herein); and/or

(2) said amino acid sequence preferably only contains amino acid substitutions, and no amino acid deletions or insertions, compared to the above amino acid sequences.

However, of the Nanobodies of the invention that comprise the combinations of CDR's mentioned above, Nanobodies comprising one or more of the CDR's listed above are particularly preferred; Nanobodies comprising two or more of the CDR's listed above are more particularly preferred; and Nanobodies comprising three of the CDR's listed above are most particularly preferred.

In these Nanobodies against human serum albumin, the Framework regions FR1 to FR4 are preferably as defined hereinabove for the Nanobodies of the invention.

Some preferred, but non-limiting examples of Nanobodies directed against human serum albumin that can be used in the present invention are listed in Table A-9 below. Some alternative serum albumin binders (against mouse serum albumin, against human serum albumin, and humanized Nanobodies against human serum albumin) are listed in the appended Tables 3, 4 and 5, respectively.

TABLE A-9

Preferred, but non-limiting examples
of albumin-binding Nanobodies

<Name, SEQ ID #; PRT (protein); ->
Sequence
<PMP 6A6(ALB-1), SEQ ID NO:34 ;PRT;->
AVQLVESGGGLVQPGNSLRLSCAASGFTFRSFGMSWVRQAPGKEPEWVSS
ISGSGSDTLYADSVKGRFTISRDNAKTTLYLQMNSLKPEDTAVYYCTIGG
SLSRSSQGTQVTVSS
<ALB-8(humanized ALB-1), SEQ ID NO:35 ;PRT;->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSS
<PMP 6A8(ALB-2), SEQ ID NO:36 ;PRT;->
AVQLVESGGGLVQGGGSLRLACAASERIFDLNLMGWYRQGPGNERELVAT
CITVGDSTNYADSVKGRFTISMDYTKQTVYLHMNSLRPEDTGLYYCKIRR
TWHSELWGQGTQVTVSS

Generally, any derivatives and/or polypeptides of the invention with increased half-life (for example pegylated Nanobodies or polypeptides of the invention, multispecific Nanobodies directed against A-Beta and (human) serum albumin, or Nanobodies fused to an Fc portion, all as described herein) have a half-life that is at least 1.5 times, preferably at least 2 times, such as at least 5 times, for example at least 10 times or more than 20 times, the half-life of the corresponding Nanobody of the invention.

Also, any derivatives or polypeptides of the invention with an increase half-life preferably have a half-life of more than 1 hour, preferably more than 2 hours, more preferably of more than 6 hours, such as of more than 12 hours, and for example of about one day, two days, one week, two weeks or three weeks, and preferably no more than 2 months, although the latter may be less critical.

Half-life can generally be defined as the time taken for the serum concentration of the polypeptide to be reduce by 50%, in vivo, for example due to degradation of the ligand and/or clearance or sequestration of the ligand by natural mechanisms. Methods for pharmacokinetic analysis and determination of half-life are familiar to those skilled in the art. Details may be found in Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2 nd Rev. ex edition (1982).

Another preferred, but non-limiting example of a multispecific polypeptide of the invention comprises at least one Nanobody of the invention and at least one Nanobody that directs the polypeptide of the invention towards, and/or that allows the polypeptide of the invention to penetrate or to enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such Nanobodies include Nanobodies that are directed towards specific cell-surface proteins, markers or epitopes of the desired organ, tissue or cell (for example cell-surface markers associated with tumor cells), and the single-domain brain targeting antibody fragments described in WO 02/057445, of which FC44 (SEQ ID NO: 189) and FC5 (SEQ ID NO: 190) as used herein are preferred examples.

In the polypeptides of the invention, the one or more Nanobodies and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser)_{z}, such as (for example (gly₄ser)₃ or (gly₃ser₂)₃, as described in WO 99/42077, hinge-like regions such as the hinge regions of naturally occurring heavy chain antibodies or similar sequences (such as those described in WO 94/04678).

Some other particularly preferred linkers are poly-alanine (such as AAA), GGGGSGGGGSGGGGSGGGGSGGGGSGGGGS (“GS30”, SEQ ID NO:32) and GGGGSGGGS (“GS9”, SEQ ID NO: 33) and with AAA and GS9 being especially preferred. Some other linker sequences are mentioned in Table 7.

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for A-beta or against the one or more of the other antigens. Based on the disclosure herein, the skilled person will be able to determine the optimal linker(s) for use in a specific polypeptide of the invention, optionally after on some limited routine experiments.

For example, in multivalent polypeptides of the invention that comprise Nanobodies directed against a multimeric antigen (such as a multimeric receptor or other protein), the length and flexibility of the linker are preferably such that it allows each Nanobody of the invention present in the polypeptide to bind to the antigenic determinant on each of the subunits of the multimer. Similarly, in a multispecific polypeptide of the invention that comprises Nanobodies directed against two or more different antigenic determinants on the same antigen (for example against different epitopes of an antigen and/or against different subunits of a multimeric receptor, channel or protein), the length and flexibility of the linker are preferably such that it allows each Nanobody to bind to its intended antigenic determinant. Again, based on the disclosure herein, the skilled person will be able to determine the optimal linker(s) for use in a specific polypeptide of the invention, optionally after on some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after on some limited routine experiments.

Finally, when two or more linkers are used in the polypeptides of the invention, these linkers may be the same or different. Again, based on the disclosure herein, the skilled person will be able to determine the optimal linkers for use in a specific polypeptide of the invention, optionally after on some limited routine experiments.

Usually, for easy of expression and production, a polypeptide of the invention will be a linear polypeptide. However, the invention in its broadest sense is not limited thererto. For example, when a polypeptide of the invention comprises three of more Nanobodies, it is possible to link them use a linker with three or more “arms”, which each “arm” being linked to a Nanobody, so as to provide a “star-shaped” construct. It is also possible, although usually less preferred, to use circular constructs.

The invention also comprises derivatives of the polypeptides of the invention, which may be essentially analogous to the derivatives of the Nanobodies of the invention. i.e. as described herein.

The invention also comprises proteins or polypeptides that “essentially consist” of a polypeptide of the invention (in which the wording “essentially consist of has essentially the same meaning as indicated hereinabove).

According to one embodiment of the invention, the polypeptide of the invention is in essentially isolated from, as defined herein.

The Nanobodies, polypeptides and nucleic acids of the invention can be prepared in a manner known per se, as will be clear to the skilled person from the further description herein.

For example, the Nanobodies and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the Nanobodies, polypeptides and nucleic acids include the methods and techniques described herein.

As will be clear to the skilled person, one particularly useful method for preparing a Nanobody and/or a polypeptide of the invention generally comprises the steps of:
the expression, in a suitable host cell or host organism (also referred to herein as a “host of the invention”) or in another suitable expression system of a nucleic acid that encodes said Nanobody or polypeptide of the invention (also referred to herein as a “nucleic acid of the invention”), optionally followed by:
isolating and/or purifying the Nanobody or polypeptide of the invention thus obtained.

In particular, such a method may comprise the steps of:
cultivating and/or maintaining a host of the invention under conditions that are such that said host of the invention expresses and/or produces at least one Nanobody and/or polypeptide of the invention; optionally followed by:
isolating and/or purifying the Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises
a) at least one nucleic acid of the invention; operably connected to
b) one or more regulatory elements, such as a promoter and optionally a suitable terminator;
   and optionally also
c) one or more further elements of genetic constructs known per se;
   in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory requences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein and/or those used in the Examples.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression bacterial cells, such as those mentioned herein and/or those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person. Reference is also made to the general background art cited above and the further references cited herein.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as those mentioned herein.

The nucleic acids of the invention and/or the genetic constructs of the invention may be used to transform a host cell or host organism, i.e. for expression and/or production of the Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example derived a cell or cell line derived from a human, from the mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The Nanobodies and polypeptides of the invention can also be introduced and expressed in one or more cells, tissues or organs of a multicellular organism, for example for prophylactic and/or therapeutic purposes (e.g. as a gene therapy). For this purpose, the nucleotide sequences of the invention may be introduced into the cells or tissues in any suitable way, for example as such (e.g. using liposomes) or after they have been inserted into a suitable gene therapy vector (for example derived from retroviruses such as adenovirus, or parvovirusses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Venna, Nature 389 (1994), 239; Isner, Lancet 348 (1996), 370-374; Muhlhauser, Circ. Res. 77 (1995), 1077-1086; Onodera, Blood 91; (1998), 30-36; Venna, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; 1 U.S. Pat. No. 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

For expression of the Nanobodies in a cell, they may also be expressed as so-called or as so-called “intrabodies”, as for example described in WO 94/02610, WO 95/22618 and U.S. Pat. No. 6,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

For production, the Nanobodies and polypeptides of the invention can for example also be produced in the milk of transgenic mammals, for example in the milk of rabbits, cows, goats or sheep (see for example U.S. Pat. No. 5,741,957, U.S. Pat. No. 5,304,489 and U.S. Pat. No. 5,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, Polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast are usually leads to a glycosylation pattern that differs from human glycosylation. Nevertheless, it should be understood that all the foregoing host cells and expression systems can be used in the invention, depending on the desired Nanobody or protein to be obtained.

Thus, according to one non-limiting embodiment of the invention, the Nanobody or polypeptide of the invention is glycosylated. According to another non-limiting embodiment of the invention, the Nanobody or polypeptide of the invention is non-glycosylated.

According to one preferred, but non-limiting embodiment of the invention, the Nanobody or polypeptide of the invention is produced in a bacterial cell, in particular a bacterial cell suitable for large scale pharmaceutical production, such as cells of the strains mentioned above.

According to another preferred, but non-limiting embodiment of the invention, the Nanobody or polypeptide of the invention is produced in a yeast cell, in particular a yeast cell suitable for large scale pharmaceutical production, such as cells of the species mentioned above.

According to yet another preferred, but non-limiting embodiment of the invention, the Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

When expression in a host cell is used to produce the Nanobodies and the proteins of the invention, the Nanobodies and proteins of the invention can be produced either intracellularly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. When eukaryotic hosts cells are used, extracellular production is usually preferred since this considerably facilitates the further isolation and downstream processing of the Nanobodies and proteins obtained. Bacterial cells such as the strains of E. coli mentioned above normally do not secrete proteins extracellularly, except for a few classes of proteins such as toxins and hemolysin, and secretory production in E. coli refers to the translocation of proteins across the inner membrane to the periplasmic space. Periplasmic production provides several advantages over cytosolic production. For example, the N-terminal amino acid sequence of the secreted product can be identical to the natural gene product after cleavage of the secretion signal sequence by a specific signal peptidase. Also, there appears to be much less protease activity in the periplasm than in the cytoplasm. In addition, protein purification is simpler due to fewer contaminating proteins in the periplasm. Another advantage is that correct disulfide bonds may form because the periplasm provides a more oxidative environment than the cytoplasm. Proteins overexpressed in E. coli are often found in insoluble aggregates, so-called inclusion bodies. These inclusion bodies may be located in the cytosol or in the periplasm; the recovery of biologically active proteins from these inclusion bodies requires a denaturation/refolding process. Many recombinant proteins, including therapeutic proteins, are recovered from inclusion bodies. Alternatively, as will be clear to the skilled person, recombinant strains of bacteria that have been genetically modified so as to secrete a desired protein, and in particular a Nanobody or a polypeptide of the invention, can be used.

Thus, according to one non-limiting embodiment of the invention, the Nanobody or polypeptide of the invention is a Nanobody or polypeptide that has been produced intracellularly and that has been isolated from the host cell, and in particular from a bacterial cell or from an inclusion body in a bacterial cell. According to another non-limiting embodiment of the invention, the Nanobody or polypeptide of the invention is a Nanobody or polypeptide that has been produced extracellularly, and that has been isolated from the medium in which the host cell is cultivated.

Some preferred, but non-limiting promoters for use with these host cells include,
for expression in E. coli: lac promoter (and derivatives thereof such as the lacUV5 promoter); arabinose promoter; left- (PL) and rightward (PR) promoter of phage lambda; promoter of the trp operon; hybrid lac/trp promoters (tac and trc); T7-promoter (more specifically that of T7-phage gene 10) and other T-phage promoters; promoter of the Tn10 tetracycline resistance gene; engineered variants of the above promoters that include one or more copies of an extraneous regulatory operator sequence;
for expression in S. cerevisiae: constitutive: ADH1 (alcohol dehydrogenase 1), ENO (enolase), CYC1 (cytochrome c iso-1), GAPDH (glyceraldehydes-3-phosphate dehydrogenase); PGK1 (phosphoglycerate kinase), PYK1 (pyruvate kinase); regulated: GAL1,10,7 (galactose metabolic enzymes), ADH2 (alcohol dehydrogenase 2), PHO5 (acid phosphatase), CUP1 (copper metallothionein); heterologous: CaMV (cauliflower mosaic virus 35S promoter);
for expression in Pichia pastoris: the AOX1 promoter (alcohol oxidase I)
for expression in mammalian cells: human cytomegalovirus (hCMV) immediate early enhancer/promoter; human cytomegalovirus (hCMV) immediate early promoter variant that contains two tetracycline operator sequences such that the promoter can be regulated by the Tet repressor; Herpes Simplex Virus thymidine kinase (TK) promoter; Rous Sarcoma Virus long terminal repeat (RSV LTR) enhancer/promoter; elongation factor 1 alpha (hEF-1 alpha) promoter from human, chimpanzee, mouse or rat; the SV40 early promoter; HIV-1 long terminal repeat promoter; Beta-actin promoter;
   Some preferred, but non-limiting vectors for use with these host cells include:
vectors for expression in mammalian cells: pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565), as well as viral-based expression systems, such as those based on adenovirus;
vectors for expression in bacterials cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen) and other baculovirus vectors
vectors for expression in plants or plant cells: for example vectors based on cauliflower mosaic virus or tobacco mosaic virus, suitable strains of Agrobacterium, or Ti-plasmid based vectors.
   Some preferred, but non-limiting secretory sequences for use with these host cells include:
for use in bacterial cells such as E. coli: PelB, Bla, OmpA, OmpC, OmpF, OmpT, StII, PhoA, PhoE, MalE, Lpp, LamB, and the like; TAT signal peptide, hemolysin C-terminal secretion signal
for use in yeast: alpha-mating factor prepro-sequence, phosphatase (phol), invertase (Suc), etc.;
for use in mammalian cells: indigenous signal in case the target protein is of eukaryotic origin; murine Ig kappa-chain V-J2-C signal peptide; etc.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell/host organism used. Also, the amino acid sequence of the invention may be glycosylated, again depending on the host cell/host organism used.

The amino acid sequence of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the amino acid sequence to be isolated).

Generally, for pharmaceutical use, the Nanobodies or polypeptides of the invention may be formulated as a pharmaceutical preparation comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one Nanobody of the invention or at least one polypeptide of the invention and at least one suitable carrier, diluent or excipient (i.e. suitable for pharmaceutical use), and optionally one or more further active substances.

Generally, the Nanobodies and polypeptides of the invention can be formulated and administered in any suitable manner known per se, for which reference is for example made to the general background art cited above (and in particular to WO 04/041862, WO 04/041863, WO 04/041865 and WO 04/041867) as well as to the standard handbooks, such as Remington's Pharmaceutical Sciences, 18^{th} Ed., Mack Publishing Company, USA (1990) or Remington, the Science and Practice of Pharmacy, 21th Edition, Lippincott Williams and Wilkins (2005).

For example, the Nanobodies and polypeptides of the inventions may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

The Nanobodies of the invention may also be administered using suitable depot or slow-release formulations (e.g. suitable for injection), using controlled-release devices for implantation under the skin, and/or using a dosing pump or other devices known per se for the administration of pharmaceutically active substances or principles. Suitable examples of such formulations and devices will be clear to the skilled person.

Also, compared to conventional antibodies or antibody fragments, one major advantage of the use of the Nanobodies and polypeptides of the invention is that they can also easily be administered via other routes than parenteral administration and can be easily formulated for such administration. For example, as described in the international application WO 04/041867 and in the further prior art referred to above, Nanobodies and Nanobody constructs may be formulated for oral, intranasal, intrapulmonary and transdermal administration.

Another embodiment of the present invention is a polypeptide construct, nucleic acid or composition as described above or a use of a polypeptide construct as described above wherein said polypeptide construct is administered intravenously, subcutaneously, orally, sublingually, topically, nasally, vaginally, rectally or by inhalation.

Another embodiment of the present invention is a method of identifying an agent that modulates platelet-mediated aggregation comprising

(a) contacting a polypeptide construct as described above with a polypeptide corresponding to its target, or a fragment thereof, in the presence and absence of a candidate modulator under conditions permitting binding between said polypeptides, and
(b) measuring the binding between the polypeptides of step (a), wherein a decrease in binding in the presence of said candidate modulator, relative to the binding in the absence of said candidate modulator identified said candidate modulator as an agent that modulate platelet-mediated aggregation.

Another embodiment of the present invention is a kit for screening for agents that modulate platelet-mediated aggregation according to the method as described above.

Another embodiment of the present invention is a method of diagnosing a disease or disorder characterised by dysfunction of platelet-mediated aggregation comprising the steps of:

(a) contacting a sample with a polypeptide construct as described above, and
(b) detecting binding of said polypeptide construct to said sample, and
(c) comparing the binding detected in step (b) with a standard, wherein a difference in binding relative to said sample is diagnostic of a disease or disorder characterised by dysfunction of platelet-mediated aggregation.

Another embodiment of the present invention is a kit for screening for diagnosing a disease or disorder characterised by dysfunction of platelet-mediated aggregation according to the method as described above.

Another embodiment of the present invention is a kit as described above comprising a polypeptide construct as described above.

By simultaneous administration means the polypeptide and thrombolytic agent are administered to a subject at the same time. For example, as a mixture or a composition comprising said components. Examples include, but are not limited to a solution administered intravenously, a tablet, liquid, topical cream, etc., wherein each preparation comprises the components of interest.

The Nanobodies of the invention may be joined to form any of the polypeptide of the invention disclosed herein comprising more than one Nanobody of the invention using methods known in the art or any future method. For example, they may be fused by chemical cross-linking by reacting amino acid residues with an organic derivatisation agent such as described by Blattler et al, Biochemistry 24, 1517-1524; EP294703.

The Nanobodies and polypeptides of the invention not only possess the advantageous characteristics of conventional antibodies, such as low toxicity and high selectivity, but they also exhibit additional properties. They are more soluble, meaning they may be stored and/or administered in higher concentrations compared with conventional antibodies. They are stable at room temperature meaning they may be prepared, stored and/or transported without the use of refrigeration equipment, conveying a cost, time and environmental savings.

A short and controllable half-life is desirable for surgical procedures, for example, which require an inhibition of platelet-mediated aggregation for a limited time period. Also, when bleeding problems occur or other complications, dosage can be lowered immediately. The polypeptides of the present invention also retain binding activity at a pH and temperature outside those of usual physiological ranges, which means they may be useful in situations of extreme pH and temperature which require a modulation of platelet-mediated aggregation, such as in gastric surgery, control of gastric bleeding, assays performed at room temperature etc. The polypeptides of the present invention also exhibit a prolonged stability at extremes of pH, meaning they would be suitable for delivery by oral administration. The polypeptides of the present invention may be cost-effectively produced through fermentation in convenient recombinant host organisms such as Escherichia coli and yeast; unlike conventional antibodies which also require expensive mammalian cell culture facilities, achievable levels of expression are high. Examples of yields of the polypeptides of the present invention are 1 to 10 mg/ml (E. coli) and up to 1 g/l (yeast). The polypeptides of the present invention also exhibit high binding affinity for a broad range of different antigen types, and ability to bind to epitopes not recognised by conventional antibodies; for example they display long CDR-based loop structures with the potential to penetrate into cavities and exhibit enzyme function inhibition. Furthermore, since binding often occurs through the CDR3 loop only, it is envisaged that peptides derived from CDR3 could be used therapeutically (Desmyter et al., J Biol Chem, 2001, 276: 26285-90).

As used herein, a functional portion refers to a Nanobody of the invention of sufficient length such that the interaction of interest is maintained with affinity of 1×10-6 M or better.

Alternatively a functional portion of a Nanobody of the invention comprises a partial deletion of the complete amino acid sequence and still maintains the binding site(s) and protein domain(s) necessary for the binding of and interaction with the target.

An aspect of the present invention is the administration of a polypeptide of the invention according to the invention can avoid the need for injection. Conventional antibody-based therapeutics have significant potential as drugs because they have exquisite specificity to their target and a low inherent toxicity, however, they have one important drawback: they are relatively unstable, and are sensitive to breakdown by proteases. This means that conventional antibody drugs cannot be administered orally, sublingually, topically, nasally, vaginally, rectally or by inhalation because they are not resistant to the low pH at these sites, the action of proteases at these sites and in the blood and/or because of their large size. They have to be administered by injection (intravenously, subcutaneously, etc.) to overcome some of these problems. Administration by injection requires specialist training in order to use a hypodermic syringe or needle correctly and safely. It further requires sterile equipment, a liquid formulation of the therapeutic polypeptide, vial packing of said polypeptide in a sterile and stable form and, of the subject, a suitable site for entry of the needle. Furthermore, subjects commonly experience physical and psychological stress prior to and upon receiving an injection.

An aspect of the present invention overcomes these problems of the prior art, by providing the polypeptides constructs of the present invention. Said constructs are sufficiently small, resistant and stable to be delivered orally, sublingually, topically, nasally, vaginally, rectally or by inhalation substantial without loss of activity. The polypeptides constructs of the present invention avoid the need for injections, are not only cost/time savings, but are also more convenient and more comfortable for the subject.

In a non-limiting example, a formulation according to the invention comprises a Nanobody or polypeptide of the invention, in the form of a gel, cream, suppository, film, or in the form of a sponge or as a vaginal ring that slowly releases the active ingredient over time (such formulations are described in EP 707473, EP 684814, U.S. Pat. No. 5,629,001).

This process can be even further enhanced by an additional aspect of the present invention—the use of active transport carriers. In this aspect of the invention, V_{HH} is fused to a carrier that enhances the transfer through the intestinal wall into the bloodstream. In a non-limiting example, this “carrier” is a second V_{HH} which is fused to the therapeutic V_{HH}. Such fusion constructs are made using methods known in the art. The “carrier” V_{HH} binds specifically to a receptor on the intestinal wall which induces an active transfer through the wall.

This process can be even further enhanced by an additional aspect of the present invention—the use of active transport carriers. In this aspect of the invention, a Nanobody or polypeptide of the invention as described herein is fused to a carrier that enhances the transfer through the intestinal wall into the bloodstream. In a non-limiting example, this “carrier” is a V_{HH} which is fused to said polypeptide. Such fusion constructs made using methods known in the art. The “carrier” V_{HH} binds specifically to a receptor on the intestinal wall which induces an active transfer through the wall.

A formulation of said Nanobody or polypeptide of the invention, for example, a cream, film, spray, drop, patch, is placed on the skin and passes through.

In another embodiment of the present invention, a Nanobody or polypeptide of the invention further comprises a carrier Nanobody of the invention (e.g. V_{HH}) which acts as an active transport carrier for transport of said Nanobody or polypeptide of the invention via the lung lumen to the blood.

A Nanobody or polypeptide of the invention further comprising a carrier that binds specifically to a receptor present on the mucosal surface (bronchial epithelial cells) resulting in the active transport of the polypeptide from the lung lumen to the blood. The carrier Nanobody of the invention may be fused to the Nanobody or polypeptide of the invention. Such fusion constructs made using methods known in the art and are describe herein. The “carrier” Nanobody of the invention binds specifically to a receptor on the mucosal surface which induces an active transfer through the surface.

Another aspect of the present invention is a method to determine which Nanobodies of the invention (e.g. V_{HH}s) are actively transported into the bloodstream upon nasal administration.

A non-limiting example of a receptor for active transport from the lung lumen to the bloodstream is the Fc receptor N (FcRn).

According to an aspect of the invention, the anti-A-beta polypeptides can be used for oral administration. Conventional antibody-based therapeutics have significant potential as drugs because they have exquisite specificity to their target and a low inherent toxicity, however, they have one important drawback: they are relatively unstable, and are sensitive to breakdown by proteases. This means that conventional antibody drugs cannot be administered orally, sublingually, topically, nasally, vaginally, rectally or by inhalation because they are not resistant to the low pH at these sites, the action of proteases at these sites and in the blood and/or because of their large size. They have to be administered by injection (intravenously, subcutaneously, etc.) to overcome some of these problems. Administration by injection requires specialist training in order to use a hypodermic syringe or needle correctly and safely. It further requires sterile equipment, a liquid formulation of the therapeutic polypeptide, vial packing of said polypeptide in a sterile and stable form and, of the subject, a suitable site for entry of the needle. Furthermore, subjects commonly experience physical and psychological stress prior to and upon receiving an injection. Nevertheless, the polypeptides of the invention may be used for administration through injection.

An aspect of the present invention overcomes these problems of the prior art, by providing the anti-A-beta polypeptides of the present invention. Said polypeptides are sufficiently small, resistant and stable to be delivered orally, sublingually, topically, nasally, vaginally, rectally or by inhalation substantial without loss of activity. The polypeptides of the present invention avoid the need for injections, are not only cost/time savings, but are also more convenient and more comfortable for the subject.

One embodiment of the present invention is an anti-A-beta polypeptide as disclosed herein for use in treating, preventing and/or alleviating the symptoms of disorders susceptible to modulation by a substance that controls A-beta which is able to pass through the gastric environment without the substance being inactivated.

As known by persons skilled in the art, once in possession of said polypeptide, formulation technology may be applied to release a maximum amount of polypeptide in the right location (in the stomach, in the colon, etc.). This method of delivery is important for treating, preventing and/or alleviating the symptoms of disorders whose targets are located in the gut system.

An aspect of the invention is a method for treating, preventing and/or alleviating the symptoms of a disorder susceptible to modulation by a substance that controls A-beta which is able to pass through the gastric environment without being inactivated, by orally administering to a subject an anti-A-beta polypeptide as disclosed herein.

Another embodiment of the present invention is a use of an anti-A-beta polypeptide as disclosed herein for the preparation of a medicament for treating, preventing and/or alleviating the symptoms of disorders susceptible to modulation by a substance that controls A-beta which is able to pass through the gastric environment without being inactivated.

An aspect of the invention is a method for delivering a substance that controls A-beta to the gut system without said substance being inactivated, by orally administering to a subject an anti-A-beta polypeptide as disclosed herein.

An aspect of the invention is a method for delivering a substance that controls A-beta to the bloodstream of a subject without the substance being inactivated, by orally administering to a subject an anti-A-beta polypeptide as disclosed herein.

Another embodiment of the present invention is an anti-A-beta polypeptide as disclosed herein, for use in treating, preventing and/or alleviating the symptoms of disorders susceptible to modulation by a substance that controls A-beta delivered to the nose, upper respiratory tract and/or lung.

In a non-limiting example, a formulation according to the invention, comprises an anti-A-beta polypeptide as disclosed herein in the form of a nasal spray (e.g. an aerosol) or inhaler. Since the polypeptide is small, it can reach its target much more effectively than therapeutic IgG molecules.

An aspect of the invention is a method for treating, preventing and/or alleviating the symptoms of disorders susceptible to modulation by a substance that controls A-beta delivered to the upper respiratory tract and lung, by administering to a subject an anti-A-beta polypeptide as disclosed herein, by inhalation through the mouth or nose.

Another embodiment of the present invention is a use of an anti-A-beta polypeptide as disclosed herein for the preparation of a medicament for treating, preventing and/or alleviating the symptoms of disorders susceptible to modulation by a substance that controls A-beta delivered to the nose, upper respiratory tract and/or lung, without said polypeptide being inactivated.

An aspect of the invention is a method for delivering a substance that controls A-beta to the nose, upper respiratory tract and lung without inactivation, by administering to the nose, upper respiratory tract and/or lung of a subject an anti-A-beta polypeptide as disclosed herein.

An aspect of the invention is a method for delivering a substance that controls A-beta to the bloodstream of a subject without inactivation by administering to the nose, upper respiratory tract and/or lung of a subject an anti-A-beta polypeptide as disclosed herein.

One embodiment of the present invention is an anti-A-beta polypeptide as disclosed herein for use in treating, preventing and/or alleviating the symptoms of disorders susceptible to modulation by a substance that controls A-beta which is able pass through the tissues beneath the tongue effectively. A formulation of said polypeptide as disclosed herein, for example, a tablet, spray, drop is placed under the tongue and adsorbed through the mucus membranes into the capillary network under the tongue.

An aspect of the invention is a method for treating, preventing and/or alleviating the symptoms of disorders susceptible to modulation by a substance that controls A-beta which is able pass through the tissues beneath the tongue effectively, by sublingually administering to a subject an anti-A-beta polypeptide as disclosed herein.

Another embodiment of the present invention is a use of an anti-A-beta polypeptide as disclosed herein for the preparation of a medicament for treating, preventing and/or alleviating the symptoms of disorders susceptible to modulation by a substance that controls A-beta which is able to pass through the tissues beneath the tongue.

An aspect of the invention is a method for delivering a substance that controls A-beta to the tissues beneath the tongue without being inactivated, by administering sublingually to a subject an anti-A-beta polypeptide as disclosed herein.

An aspect of the invention is a method for delivering a substance that controls A-beta to the bloodstream of a subject without being inactivated, by administering orally to a subject an anti-A-beta polypeptide as disclosed herein.

One embodiment of the present invention is an anti-A-beta polypeptide as disclosed herein for use in treating, preventing and/or alleviating the symptoms of disorders susceptible to modulation by a substance that controls A-beta delivered to the intestinal mucosa, wherein said disorder increases the permeability of the intestinal mucosa. Because of its small size, an anti-A-beta polypeptide as disclosed herein can pass through the intestinal mucosa and reach the bloodstream more efficiently in subjects suffering from disorders which cause an increase in the permeability of the intestinal mucosa.

An aspect of the invention is a method for treating, preventing and/or alleviating the symptoms of disorders susceptible to modulation by a substance that controls A-beta delivered to the intestinal mucosa, wherein said disorder increases the permeability of the intestinal mucosa, by orally administering to a subject an anti-A-beta polypeptide as disclosed herein.

This process can be even further enhanced by an additional aspect of the present invention—the use of active transport carriers. In this aspect of the invention, a heavy chain antibody is fused to a carrier that enhances the transfer through the intestinal wall into the bloodstream. In a non-limiting example, this “carrier” is a second a heavy chain antibody which is fused to the therapeutic a heavy chain antibody. Such fusion polypeptides are made using methods known in the art. The “carrier” a heavy chain antibody binds specifically to a receptor on the intestinal wall which induces an active transfer through the wall.

Another embodiment of the present invention is a use of an anti-A-beta polypeptide as disclosed herein for the preparation of a medicament for treating, preventing and/or alleviating the symptoms of disorders susceptible to modulation by a substance that controls A-beta delivered to the intestinal mucosa, wherein said disorder increases the permeability of the intestinal mucosa.

An aspect of the invention is a method for delivering a substance that controls A-beta to the intestinal mucosa without being inactivated, by administering orally to a subject an anti-A-beta polypeptide of the invention.

An aspect of the invention is a method for delivering a substance that controls A-beta to the bloodstream of a subject without being inactivated, by administering orally to a subject an anti-A-beta polypeptide of the invention.

This process can be even further enhanced by an additional aspect of the present invention—the use of active transport carriers. In this aspect of the invention, an anti-A-beta polypeptide as described herein is fused to a carrier that enhances the transfer through the intestinal wall into the bloodstream. In a non-limiting example, this “carrier” is a nanobody which is fused to said polypeptide. Such fusion polypeptides made using methods known in the art. The “carrier” nanobody binds specifically to a receptor on the intestinal wall which induces an active transfer through the wall.

In another embodiment of the present invention, an anti-A-beta polypeptide as disclosed herein further comprises a carrier heavy chain antibody (e.g. nanobody) which acts as an active transport carrier for transport of said polypeptide via the lung lumen to the blood.

An anti-A-beta polypeptide further comprising a carrier that binds specifically to a receptor present on the mucosal surface (bronchial epithelial cells) resulting in the active transport of the polypeptide from the lung lumen to the blood. The carrier heavy chain antibody may be fused to the polypeptide. Such fusion polypeptides made using methods known in the art and are describe herein. The “carrier” heavy chain antibody binds specifically to a receptor on the mucosal surface which induces an active transfer through the surface.

Another aspect of the present invention is a method to determine which heavy chain antibodies (e.g. nanobodies) are actively transported into the bloodstream upon nasal administration. Similarly, a naïve or immune nanobody phage library can be administered nasally, and after different time points after administration, blood or organs can be isolated to rescue phages that have been actively transported to the bloodstream. A non-limiting example of a receptor for active transport from the lung lumen to the bloodstream is the Fc receptor N (FcRn). One aspect of the invention includes the nanobodies identified by the method. Such nanobodies can then be used as a carrier nanobody for the delivery of a therapeutic nanobody to the corresponding target in the bloodstream upon nasal administration.

One embodiment of the present invention is an anti-A-beta polypeptide as disclosed herein for use in treating, preventing and/or alleviating the symptoms of disorders mediated by A-beta or dysfunction thereof, or mediated by amyloid plaque formation.

Disorders as mentioned herein include Adult Down Syndrome, Alzheimer's Disease, Amyotrophic Lateral Sclerosis/Parkinsonism Dementia Complex, Amyloid Polyneuropathy, Amyloid Cardiomyopathy, Amyloid in dialysis patients, Beta2-Microglobulin, Beta2-Amyloid deposits in muscle wasting disease, Corticobasal Degeneration, Creutzfeldt-Jacob Disease, Dementia Pugilistica, Fatal Familial Insomnia, Gerstamnn-Straussler-Scheinker Syndrome, Guam-Parkinsonism dementia complex, Hallervorden-Spatz Disease, Hereditary Cerebral Hemorrhage with Amyloidosis, Idiopathetic Myeloma, Inclusion Body Myositis, Islets of Langerhans Diabetes Type2 Insulinoma, Kuru, Medullary Carcinoma of the Thyroid, Mediterranean Fever, Muckle-Wells Syndrome, Neurovisceral Lipid Storage Disease, Parlcinson's Disease, Pick's Disease, Polyglutamine diseases including Huntington's Disease, Kennedy's Disease and all forms of Spinocerebellar Ataxia involving extended polyglutamine tracts, Progressive Supranuclear Palsy, Subacute Sclerosing Panencephalitis, Systemic Senile Amyloidosis, Scrapie.

One aspect of the invention is an anti-A-beta polypeptide as disclosed herein for use in the treatment, prevention and/or alleviation of disorders or conditions mediated by A-beta or dysfunction thereof, or mediated by amyloid plaque formation wherein said polypeptide is administered intravenously, subcutaneously, orally, sublingually, topically, nasally, vaginally, rectally or by inhalation.

Another aspect of the invention is an anti-A-beta polypeptide as disclosed herein for use in the treatment, prevention and/or alleviation of disorders or conditions mediated by A-beta or dysfunction thereof, or mediated by amyloid plaque formation.

Another aspect of the invention is the use of an anti-A-beta polypeptide as disclosed herein for the preparation of a medicament for the treatment, prevention and/or alleviation of disorders or conditions mediated by A-beta or dysfunction thereof, or mediated by amyloid plaque formation wherein said polypeptide is administered intravenously, subcutaneously, orally, sublingually, topically, nasally, vaginally, rectally or by inhalation.

Another aspect of the invention is the use of an anti-A-beta polypeptide as disclosed herein for the preparation of a medicament for the treatment, prevention and/or alleviation of disorders or conditions mediated by A-beta or dysfunction thereof, or mediated by amyloid plaque formation.

Another aspect of the invention is a method of treating, preventing and/or alleviating disorders or conditions mediated by A-beta or dysfunction thereof, or mediated by amyloid plaque formation comprising administering to a subject an anti-A-beta polypeptide as disclosed herein, wherein said polypeptide is administered intravenously, subcutaneously, orally, sublingually, topically, nasally, vaginally, rectally or by inhalation.

Another aspect of the invention is a method of treating, preventing and/or alleviating disorders or conditions mediated by A-beta or dysfunction thereof or mediated by amyloid plaque formation.

In one embodiment is an anti-A-beta polypeptide of the present invention for use as an antidote in a subject after treatment with compounds targeting A-beta.

Another embodiment of the present invention is a method and kit for detecting disorders mediated by A-beta and/or protein tau, or dysfunction thereof, or mediated by amyloid plaque formation in a subject using an anti-A-beta polypeptide and/or anti-protein tau heavy chain antibody as disclosed herein. Therefore, the methods and kits can also be useful for prescribing a treatment for a subject. Suitable treatment can be designed to delay or prevent the onset of such disorders. The present invention is also useful in monitoring the effectiveness of a prescribed treatment.

One embodiment of the present invention is a method of diagnosing a disorder mediated by A-beta and/or protein tau or dysfunction thereof, or mediated by amyloid plaque formation comprising:

(a) obtaining a sample from a subject; and
(b) determining the amount of A-beta and/or tau in the sample using an A-beta polypeptide and/or anti-protein tau heavy chain antibody of the present invention.

Another embodiment of the present invention is a method of diagnosing a disorder mediated by A-beta and/or protein tau of dysfunction thereof, or mediated by amyloid plaque formation comprising:

(a) contacting a sample with an anti-A-beta polypeptide and/or anti-protein tau heavy chain antibody as described above,
(b) detecting binding of said polypeptide or antibody to said sample, and
(c) comparing the binding detected in step (b) with a standard, wherein a difference in binding relative to said sample is diagnostic of a disorder characterised by the formation of amyloid plaque or neurofibrillary tangle.

Another embodiment of the present invention is a method of diagnosing a disorder mediated by A-beta and/or protein tau or dysfunction thereof, or mediated by amyloid plaque formation comprising:

(a) contacting a sample with an anti-A-beta polypeptide and/or anti-protein tau heavy chain antibody as described above, and
(b) determining the amount of A-beta and/or protein tau in the sample
(c) comparing the amount determined in step (b) with a standard, wherein a difference in amount relative to said sample is diagnostic of a disorder or disorder characterised by amyloid plaque formation or neurofibrillary tangle.

In one embodiment of the present invention, a sample is obtained, or collected, from a subject to be tested for a disorder mediated by A-beta and/or protein tau or dysfunction thereof or mediated by amyloid plaque formation. The subject may or may not be suspected of having a such disorder. A sample is any specimen obtained from the subject that can be used to measure the amount of native A-beta and/or protein tau. A preferred sample is a bodily fluid (preferably CSF) that can be used to measure the amount of A-beta and/or protein tau. Those skilled in the art can readily identify appropriate samples.

As used herein, the term “contacting” refers to the introduction of a sample putatively containing an A-beta or protein tau to an anti-A-beta polypeptide or anti-protein tau heavy chain antibody respectively, for example, by combining or mixing the sample with the respective polypeptide(s). When A-beta and/or protein tau are present in the sample, a complex is then formed; such complex can be detected. Detection can be qualitative, quantitative, or semi-quantitative. Binding A-beta and/or protein tau in the sample to the respective anti-A-beta polypeptide or anti-protein tau heavy chain antibody is accomplished under conditions suitable to form a complex. Such conditions (e.g. appropriate concentrations, buffers, temperatures, reaction times) as well as methods to optimize such conditions are known to those skilled in the art. Binding can be measured using a variety of methods standard in the art including, but not limited to, enzyme immunoassays (e.g., ELISA), immunoprecipitations, immunoblot assays and other immunoassays as described, for example, in Sambrook et al., supra, and Harlow et al., Antibodies, a Laboratory Manual (Cold Spring Harbor Labs Press, 1988). These references also provide examples of complex formation conditions.

In one embodiment, the aforementioned complex can be formed in solution. In another embodiment, the aforementioned complex can be formed in which one component (e.g. A-beta, protein tau, anti-A-beta polypeptide, anti-protein tau heavy chain antibody) is immobilized on (e.g., coated onto) a substrate. Immobilization techniques are known to those skilled in the art. Suitable substrate materials include, but are not limited to, plastic, glass, gel, celluloid, fabric, paper, and particulate materials. Examples of substrate materials include, but are not limited to, latex, polystyrene, nylon, nitrocellulose, agarose, cotton, PVDF (poly-vinylidene-fluoride), and magnetic resin. Suitable shapes for substrate material include, but are not limited to, a well (e.g., microtiter dish well), a microtiter plate, a dipstick, a strip, a bead, a lateral flow apparatus, a membrane, a filter, a tube, a dish, a celluloid-type matrix, a magnetic particle, and other particulates. Particularly preferred substrates include, for example, an ELISA plate, a dipstick, an immunodot strip, a radioimmunoassay plate, an agarose bead, a plastic bead, a latex bead, a sponge, a cotton thread, a plastic chip, an immunoblot membrane, an immunoblot paper and a flow-through membrane. In one embodiment, a substrate, such as a particulate, can include a detectable marker. For descriptions of examples of substrate materials, see, for example, Kemeny, D. M. (1991) A Practical Guide to ELISA, Pergamon Press, Elmsford, N.Y. pp 33-44, and Price, C. and Newman, D. eds. Principles and Practice of Immunoassay, 2nd edition (1997) Stockton Press, NY, N.Y., both of which are incorporated herein by reference in their entirety.

In a preferred embodiment, an anti-A-beta polypeptide and/or anti-protein tau heavy chain antibody is immobilized on a substrate, such as a microtiter dish well, a dipstick, an immunodot strip, or a lateral flow apparatus. A sample collected from a subject is applied to the substrate and incubated under conditions suitable (i.e., sufficient) to allow for complex formation bound to the substrate.

In accordance with the present invention, once formed, a complex is detected. As used herein, the term “detecting complex formation” refers to identifying the presence of anti-A-beta polypeptide complexed to A-beta and/or anti-protein tau heavy chain antibody complexed to protein tau. If complexes are formed, the amount of complexes formed can, but need not be, quantified. Complex formation, or selective binding, can be measured (i.e., detected, determined) using a variety of methods standard in the art (see, for example, Sambrook et al. supra.), examples of which are disclosed herein. A complex can be detected in a variety of ways including, but not limited to use of one or more of the following assays: an enzyme-linked immunoassay, a competitive enzyme-linked immunoassay, a radioimmunoassay, a fluorescence immunoassay, a chemiluminescent assay, a lateral flow assay, a flow-through assay, an agglutination assay, a particulate-based assay (e.g., using particulates such as, but not limited to, magnetic particles or plastic polymers, such as latex or polystyrene beads), an immunoprecipitation assay, a BioCore assay (e.g., using colloidal gold), an immunodot assay (e.g., CMG's Immunodot System, Fribourg, Switzerland), and an immunoblot assay (e.g., a western blot), an phosphorescence assay, a flow-through assay, a particulate-based assay, a chromatography assay, a PAGE-based assay, a surface plasmon resonance assay, a spectrophotometric assay and an electronic sensory assay. Such assays are well known to those skilled in the art.

Assays can be used to give qualitative or quantitative results depending on how they are used. The assay results can be based on detecting the entire A-beta and/or protein tau molecule or fragments, degradation products or reaction products thereof. Some assays, such as agglutination, particulate separation, and immunoprecipitation, can be observed visually (e.g., either by eye or by a machines, such as a densitometer or spectrophotometer) without the need for a detectable marker.

In other assays, conjugation of a detectable marker to the anti-A-beta polypeptide, anti-protein tau heavy chain antibody or their targets aids in detecting complex formation. For example, a detectable marker can be conjugated to the anti-A-beta polypeptide, or anti-protein tau heavy chain antibody at a site that does not interfere with their ability to bind their respective targets. Methods of conjugation are known to those of skill in the art. Examples of detectable markers include, but are not limited to, a radioactive label, a fluorescent label, a chemiluminescent label, a chromophoric label, an enzyme label, a phosphorescent label, an electronic label; a metal sol label, a colored bead, a physical label, or a ligand. A ligand refers but are not limited to, fluorescein, a radioisotope, a phosphatase (e.g., alkaline phosphatase), biotin, avidin, a peroxidase (e.g., horseradish peroxidase), beta-galactosidase, and biotin-related compounds or avidin-related compounds (e.g., streptavidin or ImmuunoPure NeutrAvidin).

The present invention can further comprise one or more layers and/or types of secondary molecules or other binding molecules capable of detecting the presence of an indicator molecule. For example, an untagged (i.e., not conjugated to a detectable marker) secondary antibody that selectively binds to an anti-A-beta polypeptide or anti-protein tau heavy chain antibody can be bound to a tagged tertiary antibody that selectively binds to the secondary antibody. Suitable secondary antibodies, tertiary antibodies and other secondary or tertiary molecules can be readily selected by those skilled in the art. Preferred tertiary molecules can also be selected by those skilled in the art based upon the characteristics of the secondary molecule. The same strategy can be applied for subsequent layers.

Depending on the assay, a developing agent is added and the substrate is submitted to a detection device for analysis. In some protocols, washing steps are added after one or both complex formation steps in order to remove excess reagents. If such steps are used, they involve conditions known to those skilled in the art such that excess reagents are removed but the complex is retained.

Once the level of A-beta and/or protein tau has been measured, an assessment of whether a disorder mediated by A-beta and/or protein tau, or dysfunction thereof or mediated by amyloid plaque formation is present can then be made. Assessing the presence of such disorder means comparing the level of A-beta and/or protein tau in the test sample to the level found in healthy subjects. The presence of A-beta and/or protein tau in the sample, in the absence of changes in neural function, is indicative of such disorder.

A diagnostic kit according to the invention comprises all the necessary means and media for performing the detection of A-beta and/or protein tau or fragment thereof by interaction an anti-A-beta polypeptide (for example, a polypeptide comprising at least one Nanobody or polypeptide as described herein) and/or anti-protein tau heavy chain antibody. The kit is useful for diagnosis of disorders or disorders mediated by A-beta, protein tau, dysfunction thereof or by the formation of amyloid plaque.

According to one aspect of the invention, a diagnostic kit comprises one or more anti-A-beta Nanobodies or polypeptides of the invention as described herein. According to one aspect of the invention, a diagnostic kit comprises one or more anti-protein tau Nanobodies of the invention.

According to another aspect of the invention, a diagnostic kit comprises one or more recombinant cells of the invention, comprising and expressing the nucleotide sequence encoding an anti-A-beta polypeptide. According to another aspect of the invention, a diagnostic kit comprises one or more recombinant cells of the invention, comprising and expressing the nucleotide sequence encoding an anti-protein tau heavy chain antibody.

Kits useful according to the invention can comprise an isolated anti-A-beta polypeptide and/or, anti-protein tau heavy chain antibody a homologue thereof, or a functional portion thereof. A kit according to the invention can comprise cells transformed to express said polypeptide.

Kits useful according to the invention can include an isolated A-beta, or fragment thereof. Alternatively, or in addition, a kit can comprise cells transformed to express A-beta, or fragment thereof. In a further embodiment, a kit according to the invention can comprise a polynucleotide encoding A-beta, or fragment thereof. In a still further embodiment, a kit according to the invention may comprise the specific primers useful for amplification of A-beta, or fragment thereof.

All kits according to the invention will comprise the stated items or combinations of items and packaging materials therefore. Kits will also include instructions for use.

A-beta, protein tau, anti-A-beta polypeptide and/or anti-protein tau heavy chain antibody may be supplied immobilised, for example, on a microtitre plate, on a glass chip suitable for high-throughput screening, on magnetic beads, or on an insoluble solid support.

The polypeptides of the invention are administered in a therapeutically and/or prohylactically effective amount, sufficient to achieve the desired therapeutic and/or prophylactic action, as a single dose or multiple doses, e.g. once or more daily over one or more days.

In general, “therapeutically effective amount”, “therapeutically effective dose” and “effective amount” means the amount needed to achieve the desired result or results (treating or preventing A-beta). One of ordinary skill in the art will recognize that the potency and, therefore, an “effective amount” can vary for the various compounds that inhibit A-beta used in the invention. One skilled in the art can readily assess the potency of the compound.

As used herein, the term “compound” refers the anti-A-beta Nanobodies or polypeptides disclosed herein, or to a nucleic acid capable of encoding said polypeptide, salts of said polypeptides, or said polypeptide comprising one or more derivatised amino acids.

By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained.

Amounts needed to achieve a therapeutically effective dose will depend upon the severity of the disease and the general state of the patient's own immune system, but generally range from 0.005 to 5.0 mg per kilogram of body weight, preferrably doses of 0.05 to 2.0 mg/kg/dose. For prophylactic applications, compositions containing the polypeptides of the invention or cocktails thereof may also be administered in similar or slightly lower dosages.

The invention disclosed herein is useful for treating or preventing conditions mediated by A-beta or dysfunction thereof, or mediated by amyloid plaque formation, in a subject and comprising administering a pharmaceutically effective amount of a compound or composition according to the invention.

One aspect of the present invention is the use of compounds of the invention for treating or preventing a condition mediated by A-beta or dysfunction thereof, or mediated by amyloid plaque formation, in a subject and comprising administering a pharmaceutically effective amount of a compound in combination with another, such as, for example, an agent capable of inhibiting one or more enzymes involved in formation of A-beta fragments.

One aspect of the present invention is the use of compounds of the invention for treating or preventing a condition mediated by A-beta or dysfunction thereof, or mediated by amyloid plaque formation, in a subject and comprising administering a pharmaceutically effective amount of a compound in combination with another, such as, for example, an anti-tangle agent.

The present invention is not limited to the administration of formulations comprising a single compound of the invention. It is within the scope of the invention to provide combination treatments wherein a formulation is administered to a patient in need thereof that comprises more than one compound of the invention.

Conditions mediated by A-beta or dysfunction thereof, or mediated by amyloid plaque formation include, but are not limited to, those described above in the present application.

The compound useful in the present invention can be formulated as pharmaceutical compositions and administered to a mammalian host, such as a human patient or a domestic animal in a variety of forms adapted to the chosen route of administration, i.e., orally or parenterally, by intra-nasally by inhalation, intravenous, intramuscular, topical or subcutaneous routes.

The compound of the present invention can also be administered using gene therapy methods of delivery. See, e.g., U.S. Pat. No. 5,399,346, which is incorporated by reference in its entirety. Using a gene therapy method of delivery, primary cells transfected with the gene for a polypeptide of the present invention can additionally be transfected with tissue specific promoters to target specific organs, tissue, grafts, tumors, or cells.

Thus, a present compound may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet.

For oral therapeutic administration, a compound may be combined with one or more excipients and used in the form of ingestible tablets, buccal tablets, troches, capsules, elixirs, suspensions, syrups, wafers, and the like. Such compositions and preparations may contain at least 0.1% w/w of compound. The percentage of the compositions and preparations may, of course, be varied and may conveniently be between about 2 to about 60% w/w of a given unit dosage form. The amount of compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain an active polypeptide, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compound may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the present compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compound to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound can be determined by comparing its in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. Also the dosage of the compound varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

In a further aspect, the present invention provides one or more nucleic acid molecules encoding a heavy chain antibody as herein defined.

The multivalent or multispecific heavy chain antibody may be encoded on a single nucleic acid molecule; alternatively, each heavy chain antibody may be encoded by a separate nucleic acid molecule. Where the multivalent or multispecific heavy chain antibody is encoded by a single nucleic acid molecule, the Nanobodies forming part of it may be expressed as a fusion polypeptide, in the manner of a scFv molecule, or may be separately expressed and subsequently linked together, for example using chemical linking agents. Multivalent or multispecific Nanobodies expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression.

In a further aspect the present invention provides a vector comprising nucleic acid encoding a polypeptide according to the present invention.

In a yet further aspect, the present invention provides a host cell transfected with a vector encoding a polypeptide according to the present invention.

Expression from such a vector may be configured to produce, for example on the surface of a bacteriophage particle, Nanobodies for selection. This allows selection of displayed Nanobodies and thus selection of polypeptides using the method of the present invention.

The present invention further provides a kit comprising at least a polypeptide according to the present invention.

A cell that is useful according to the invention are any bacterial cells such as for example E. coli, yeast cells such as for example S. cerevisiae and P. pastoris, insect cells, mammalian cells or molds comprising those belonging to the genera Aspergillus or Trichoderma.

A cell that is useful according to the invention can be any cell into which a nucleic acid sequence encoding a Nanobody or polypeptide of the invention or an anti-A-beta Nanobody or polypeptide according to the invention can be introduced such that the polypeptide is expressed at natural levels or above natural levels, as defined herein. Preferably a polypeptide of the invention that is expressed in a cell exhibits normal or near normal pharmacology, as defined herein. Most preferably a polypeptide of the invention that is expressed in a cell comprises the nucleotide sequence capable of encoding Nanobodies and polypeptides according to the invention.

According to a preferred embodiment of the present invention, a cell is selected from the group consisting of COS7-cells, a CHO cell, a LM (TK-) cell, a NIH-3T3 cell, HEK-293 cell, K-562 cell or a 1321N1 astrocytoma cell but also other transfectable cell lines.

Imaging techniques can offer such diagnostic power. Conventional CT and MR imaging are primarily used to rule out other cases of dementia and to assess the degree of brain atrophy. SPECT, PET and fMRI have greater potential in identifying subtle pathologic changes during earlier stages of the disorder. The combination of SPECT, PET or MRI with labeled anti-A-beta polypeptide will allow ‘A-beta brain scans’ and individual risk assessment for each patient.

One aspect of the present invention is an anti-A-beta polypeptide as disclosed herein further comprising one or more imaging agents. Imaging agents are any suitable for in vivo use, including, but not limited to 99 mTc, 111Indium, 123Iodine. Other imaging agents suitable for magnetic resonance imaging include paramagnetic compounds, MR paramagnetic chelates. Other imaging agents include optical dyes.

Another aspect of the present invention is a use of an anti-A-beta polypeptide further comprising one or more imaging agents, for in vivo imaging.

The anti-A-beta polypeptides as described above may further comprise one or more anti-protein tau Nanobodies for the simultaneous imaging of A-beta and protein tau.

The anti-A-beta polypeptide may be labeled with imaging agents using methods known in the art.

It is an aspect of the invention that the labelled polypeptides are incorporated in microparticles, ultrasound bubbles, microspheres, emulsions, or liposomes. Such preparations allow for a more efficient delivery.

In another aspect, the invention relates to a method for the prevention and/or treatment of at least one disease or disorder associated with A-beta, at least one disease and disorder associated with the undesired formation or build up of amyloid plaques, and/or at least one neurodegenerative disease said method comprising administering, to a subject in need thereof, a pharmaceutically active amount of a Nanobody of the invention, of a polypeptide of the invention, and/or of a pharmaceutical composition comprising the same.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

The invention also relates to a method for the prevention and/or treatment of at least one disease or disorder that can be prevented and/or treated by administering a Nanobody or polypeptide of the invention to a patient, said method comprising administering, to a subject in need thereof, a pharmaceutically active amount of a Nanobody of the invention, of a polypeptide of the invention, and/or of a pharmaceutical composition comprising the same.

The invention further relates to a method for the prevention and/or treatment of at least one disease or disorder that can be prevented and/or treated by modulating, reducing and/or reversing the (undesired) formation or build-up of A-beta and/or of amyloid plaques in a patient, said method comprising administering, to a subject in need thereof, a pharmaceutically active amount of a Nanobody of the invention, of a polypeptide of the invention, and/or of a pharmaceutical composition comprising the same.

In particular, the invention relates to a method for the prevention and/or treatment of at least one neurodegenerative disease or disorder, said method comprising administering, to a subject in need thereof, a pharmaceutically active amount of a Nanobody of the invention, of a polypeptide of the invention, and/or of a pharmaceutical composition comprising the same.

More in particular, the invention relates to a method for the prevention and/or treatment of at least one disease or disorder chosen from the group consisting of the diseases and disorders listed herein, said method comprising administering, to a subject in need thereof, a pharmaceutically active amount of a Nanobody of the invention, of a polypeptide of the invention, and/or of a pharmaceutical composition comprising the same.

According to one specific embodiment, the invention relates to a method for the prevention and/or treatment of Alzheimer's disease, said method comprising administering, to a subject in need thereof, a pharmaceutically active amount of a Nanobody of the invention, of a polypeptide of the invention, and/or of a pharmaceutical composition comprising the same.

In another specific embodiment, the invention relates to a method for the prevention and/or treatment of cognitive decline, and/or of restoring cognitive function and/or of improving cognitive function, said method comprising administering, to a subject in need thereof, a pharmaceutically active amount of a Nanobody of the invention, of a polypeptide of the invention, and/or of a pharmaceutical composition comprising the same.

In another embodiment, the invention relates to a method for immunotherapy, and in particular for passive immunotherapy, which method comprises administering, to a subject suffering from or at risk of the diseases and disorders mentioned herein, a pharmaceutically active amount of a Nanobody of the invention, of a polypeptide of the invention, and/or of a pharmaceutical composition comprising the same.

Thus, for example, the method of the invention may be used in passive immunotherapy for delaying the onset of, slowing the progress of, and/or reversing, neurodegenerative diseases such as AD and the diseases and disorders mentioned herein; in passive immunotherapy for delaying the onset of, slowing the progress of, and/or reversing the symptoms associated therewith such as cognitive decline; in passive immunotherapy for preventing, slowing, reducing and/or reversing the deleterious accumulation of A-beta; and/or in passive immunotherapy for preventing the formation of, slowing the growth of, reducing the size of, and/or clearing up amyloid plaques (e.g. associated with AD).

In the above methods, the Nanobodies and/or polypeptides of the invention and/or the compositions comprising the same can be administered in any suitable manner, depending on the specific pharmaceutical formulation or composition to be used. Thus, the Nanobodies and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factorse well known to the clinician.

The Nanobodies and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific Nanobody or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more Nanobodies and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific Nanobody and polypeptide of the invention to be used, the specific route of administration and the specific pharmaceutical formulation or composition used, the Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single Nanobody or polypeptide of the invention will be used. It is however within the scope of the invention to use two or more Nanobodies and/or polypeptides of the invention in combination.

The Nanobodies and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the Nanobodies and polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician. Some preferred, but non-limiting examples include the active substances and principles (i.e. small molecules and biologicals such as antibodies and antibody fragments) currently on the market or in clinical development for the prevention and treatment of the diseases and disorders mentioned herein (whether active on A-beta and/or on active on another relevant target or biological pathway), such as cholinesterase inhibitors (for example Donepezil (Aricept™); Rivastigmine (Exelon™); Galantamine (Reminyl™); Tacrine (Cognex™)), NMDA antagonists (for example Memantine (Namenda™; Exura™)), inhibitors of secretases such as beta-secretase (BACE) and gamma-secretase, and other agents for preventing or treating neurodegenerative diseases and a decline in cognitive function.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are administered to be simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of a Nanobody or polypeptide of the invention in the preparation of a pharmaceutical composition for prevention and/or treatment of at least one disease or disorder associated with A-beta, at least one disease and disorder associated with the undesired formation or build up of amyloid plaques, and/or at least one neurodegenerative disease.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

The invention also relates to the use of a Nanobody or polypeptide of the invention in the preparation of a pharmaceutical composition for the prevention and/or treatment of at least one disease or disorder that can be prevented and/or treated by administering a Nanobody or polypeptide of the invention to a patient.

The invention in particular relates to the use of a Nanobody or polypeptide of the invention in the preparation of a pharmaceutical composition for the prevention and/or treatment of at least one disease or disorder that can be prevented and/or treated by modulating, reducing and/or reversing the (undesired) formation or build-up of A-beta and/or of amyloid plaques in a patient.

More in particular, the invention relates to the use of a Nanobody or polypeptide of the invention in the preparation of a pharmaceutical composition for the prevention and/or treatment of at least one neurodegenerative disease or disorder, and in particular for the prevention and treatment of one or more of the diseases and disorders listed herein.

A very specific aspect of the invention relates to the use of a Nanobody or polypeptide of the invention in the preparation of a pharmaceutical composition for the prevention and/or treatment of Alzheimer's disease.

The invention further relates to the use of a Nanobody or polypeptide of the invention in the preparation of a pharmaceutical composition for the prevention and/or treatment of cognitive decline, and/or of restoring cognitive function and/or of improving cognitive function.

The invention further relates to the use of a Nanobody or polypeptide of the invention in the preparation of a pharmaceutical composition for immunotherapy, and in particular for passive immunotherapy, and more in particular for passive immunotherapy for delaying the onset of, slowing the progress of, and/or reversing, neurodegenerative diseases such as AD and the diseases and disorders mentioned herein; in passive immunotherapy for delaying the onset of, slowing the progress of, and/or reversing the symptoms associated therewith such as cognitive decline; in passive immunotherapy for preventing, slowing, reducing and/or reversing the deleterious accumulation of A-beta; and/or in passive immunotherapy for preventing the formation of, slowing the growth of, reducing the size of, and/or clearing up amyloid plaques (e.g. associated with AD).

Again, in such a pharmaceutical composition, the one or more Nanobodies or polypeptides of the invention may also be suitably combined with one or more other active principles, such as those Finally, although the use of the Nanobodies of the invention (as defined herein) and of the polypeptides of the invention is much preferred, it will be clear that on the basis of the description herein, the skilled person will also be able to design and/or generate, in an analogous manner, other (single) domain antibodies against A-beta, as well as polypeptides comprising such (single) domain antibodies (in which the terms “domain antibody” and “single domain antibody” have their usual meaning in the art, see for example the prior art referred to herein).

Thus, one further aspect of the invention relates to domain antibodies or single domain antibodies against A-beta, and to polypeptides that comprise at least one such (single) domain antibody and/or that essentially consist of such a (single) domain antibody.

In particular, such a (single) domain antibody against A-beta may comprise 3 CDR's, in which said CDR's are as defined above for the Nanobodies of the invention. For example, such (single) domain antibodies may be the single domain antibodies known as “dAb's”, which are for example as described by Ward et al, supra, but which have CDR's that are as defined above for the Nanobodies of the invention. However, as mentioned above, the use of such “dAb's” will usually have several disadvantages compared to the use of the corresponding Nanobodies of the invention. Thus, any (single) domain antibodies against A-beta according to this aspect of the invention will preferably have framework regions that provide these (single) domain antibodies against A-beta with properties that make them substantially equivalent to the Nanobodies of the invention.

This aspect of the invention also encompasses nucleic acids that encode such (single) domain antibodies and/or polypeptides, compositions that comprise such (single) domain antibodies, polypeptides or nucleic acids, host cells that (can) express such (single) domain antibodies or polypeptides, and methods for preparing and using such (single) domain antibodies, polypeptides or nucleic acids, which may be essentially analogous to the polypeptides, nucleic acids, compositions, host cells, methods and uses described above for the Nanobodies of the invention.

Furthermore, it will also be clear to the skilled person that it may be possible to “graft” one or more of the CDR's mentioned above for the Nanobodies of the invention onto other “scaffolds”, including but not limited to human scaffolds or non-immunoglobulin scaffolds. Suitable scaffolds and techniques for such CDR grafting will be clear to the skilled person and are well known in the art, see for example U.S. Pat. No. 6,180,370, WO 01/27160, EP 0 605 522, EP 0 460 167, U.S. Pat. No. 6,054,297, Nicaise et al., Protein Science (2004), 13:1882-1891; Ewert et al., Methods, 2004 October; 34(2):184-199; Kettleborough et al., Protein Eng. 1991 October; 4(7): 773-783; O'Brien and Jones, Methods Mol. Biol. 2003:207:81-100; and Skerra, J. Mol. Recognit. 2000:13:167-187, and Saerens et al., J. Mol. Biol. 2005 Sep. 23; 352(3):597-607, and the further references cited therein. For example, techniques known per se for grafting mouse or rat CDR's onto human frameworks and scaffolds can be used in an analogous manner to provide chimeric proteins comprising one or more of the CDR's of the Nanobodies of the invention and one or human framework regions or sequences.

Thus, in another embodiment, the invention comprises a chimeric polypeptide comprising at least one CDR sequence chosen from the group consisting of CDR1 sequences, CDR2 sequences and CDR3 sequences mentioned herein for the Nanobodies of the invention. Preferably, such a chimeric polypeptide comprises at least one CDR sequence chosen from the group consisting of the CDR3 sequences mentioned herein for the Nanobodies of the invention, and optionally also at least one CDR sequence chosen from the group consisting of the CDR1 sequences and CDR2 sequences mentioned herein for the Nanobodies of the invention. For example, such a chimeric polypeptide may comprise one CDR sequence chosen from the group consisting of the CDR3 sequences mentioned herein for the Nanobodies of the invention, one CDR sequence chosen from the group consisting of the CDR1 sequences mentioned herein for the Nanobodies of the invention and one CDR sequence chosen from the group consisting of the CDR1 sequences and CDR2 sequences mentioned herein for the Nanobodies of the invention. The combinations of CDR's that are mentioned herein as being preferred for the Nanobodies of the invention will usually also be preferred for these chimeric polypeptides.

In said chimeric polypeptides, the CDR's may be linked to further amino acid sequences and/or may be linked to each other via amino acid sequences, in which said amino acid sequences are preferably framework sequences or are amino acid sequences that act as framework sequences, or together form a scaffold for presenting the CDR's.

Reference is again made to the prior art mentioned in the last paragraph. According to one preferred embodiment, the amino acid sequences are human framework sequences, for example V_{H}3 framework sequences. However, non-human, synthetic, semi-synthetic or non-immunoglobulin framework sequences may also be used. Preferably, the framework sequences used are such that (1) the chimeric polypeptide is capable of binding A-beta, i.e. with an affinity that is at least 1%, preferably at least 5%, more preferably at least 10%, such as at least 25% and up to 50% or 90% or more of the affinity of the corresponding Nanobody of the invention; (2) the chimeric polypeptide is suitable for pharmaceutical use; and (3) the chimeric polypeptide is preferably essentially non-immunogenic under the intended conditions for pharmaceutical use (i.e. indication, mode of administration, dosis and treatment regimen) thereof (which may be essentially analogous to the conditions described herein for the use of the Nanobodies of the invention).

According to one non-limiting embodiment, the chimeric polypeptide comprises at least two CDR sequences (as mentioned above) linked via at least one framework sequence, in which preferably at least one of the two CDR sequences is a CDR3 sequence, with the other CDR sequence being a CDR1 or CDR2 sequence. According to a preferred, but non-limiting embodiment, the chimeric polypeptide comprises at least two CDR sequences (as mentioned above) linked at least two framework sequences, in which preferably at least one of the three CDR sequences is a CDR3 sequence, with the other two CDR sequences being CDR1 or CDR2 sequences, and preferably being one CDR1 sequence and one CDR2 sequence. According to one specifically preferred, but non-limiting embodiment, the chimeric polypeptides have the structure FR1′-CDR1-FR2′-CDR2-FR3′-CDR3-FR4′, in which CDR1, CDR2 and CDR3 are as defined herein for the CDR's of the Nanobodies of the invention, and FR1′, FR2′, FR3′ and FR4′ are framework sequences. FR1′, FR2′, FR3′ and FR4′ may in particular be Framework 1, Framework 2, Framework 3 and Framework 4 sequences, respectively, of a human antibody (such as V_{H}3 sequences) and/or parts or fragments of such Framework sequences. It is also possible to use parts or fragments of a chimeric polypeptide with the structure FR1′-CDR1-FR2′-CDR2-FR3′-CDR3-FR4. Preferably, such parts or fragments are such that they meet the criteria set out in the preceding paragraph.

The invention also relates to proteins and polypeptides comprising and/or essentially consisting of such chimeric polypeptides, to nucleic acids encoding such proteins or polypeptides; to methods for preparing such proteins and polypeptides; to host cells expressing or capable of expressing such proteins or polypeptides; to compositions, and in particular to pharmaceutical compositions, that comprise such proteins or polypeptides, nucleic acids or host cells; and to uses of such proteins or polypeptides, such nucleic acids, such host cells and/or such compositions, in particular for prophylactic, therapeutic or diagnostic purposes, such as the prophylactic, therapeutic or diagnostic purposes mentioned herein. For example, such proteins, polypeptides, nucleic acids, methods, host cells, compositions and uses may be analogous to the proteins, polypeptides, nucleic acids, methods, host cells, compositions and use described herein for the Nanobodies of the invention.

It should also be noted that, when the Nanobodies of the inventions contain one or more other CDR sequences than the preferred CDR sequences mentioned above, these CDR sequences can be obtained in any manner known per se, for example from Nanobodies (preferred), V_{H} domains from conventional antibodies (and in particular from human antibodies), heavy chain antibodies, conventional 4-chain antibodies (such as conventional human 4-chain antibodies) or other immunoglobulin sequences directed against A-beta. Such immunoglobulin sequences directed against A-beta can be generated in any manner known per se, as will be clear to the skilled person, i.e. by immunization with A-beta or by screening a suitable library of immunoglobulin sequences with A-beta, or any suitable combination thereof. Optionally, this may be followed by techniques such as random or site-directed mutagenesis and/or other techniques for affinity maturation known per se. Suitable techniques for generating such immunoglobulin sequences will be clear to the skilled person, and for example include the screening techniques reviewed by Hoogenboom, Nature Biotechnology, 23, 9, 1105-1116 (2005). Other techniques for generating immunoglobulins against a specified target include for example the Nanoclone technology (as for example described in the non-prepublished U.S. provisional patent application 60/648,922), so-called SLAM technology (as for example described in the European patent application 0 542 810), the use of transgenic mice expressing human immunoglobulins or the well-known hybridoma techniques (see for example Larrick et al, Biotechnology, Vol. 7, 1989, p. 934). All these techniques can be used to generate immunoglobulins against A-beta, and the CDR's of such immunoglobulins can be used in the Nanobodies of the invention, i.e. as outlined above. For example, the sequence of such a CDR can be determined, synthesized and/or isolated, and inserted into the sequence of a Nanobody of the invention (e.g. so as to replace the corresponding native CDR), all using techniques known per se such as those described herein, or Nanobodies of the invention containing such CDR's (or nucleic acids encoding the same) can be synthesized de novo, again using the techniques mentioned herein.

The invention will now be further described by means of the following non-limiting examples and figures, in which the Figures show:

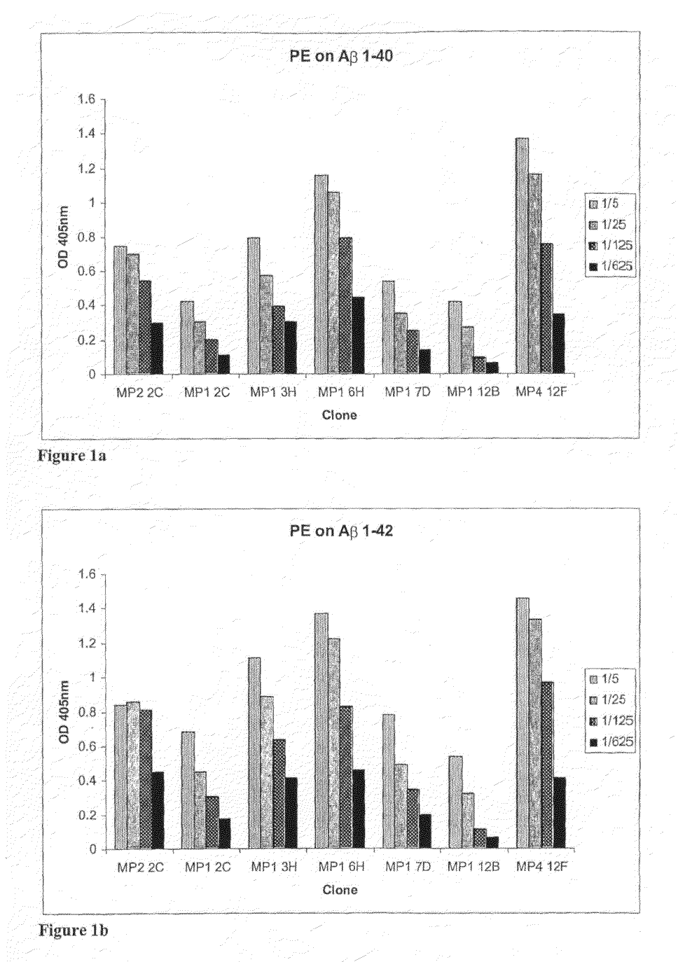

FIG. 1: Binding to solid phase coated synthetic peptides Aβ40 (FIG. 1a) and Aβ42 (FIG. 1b). Crude periplasmic extracts of seven nanobodies, at ⅕, 1/25, 1/125 and 1/625 dilution, were added to individual wells of microplates. Signals were measured at 405 nm, 5 minutes after adding 100 microliter of the chromogenic substrate (2% para nitrophenyl phosphate in pH 9.6 buffer).

FIG. 2: Binding to solid phase coated synthetic peptides Aβ40 (FIG. 2a) and Aβ42 (FIG. 2b) of purified nanobodies at different concentrations starting at 10 micrograms/ml. Signals were measured at 405 nm.

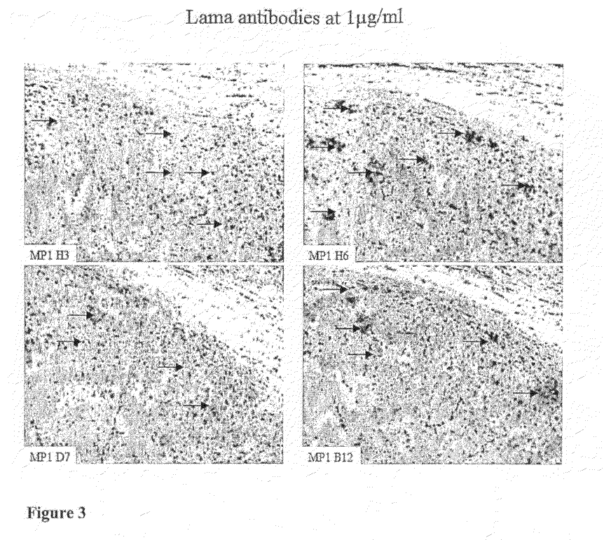

FIG. 3: Detection of amyloid plaques in transgenic mouse brain. Arrows point to zones of intense brown staining.

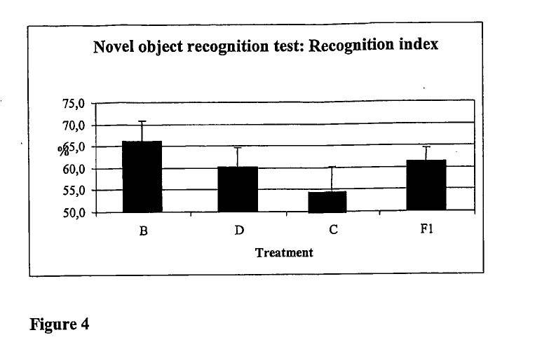

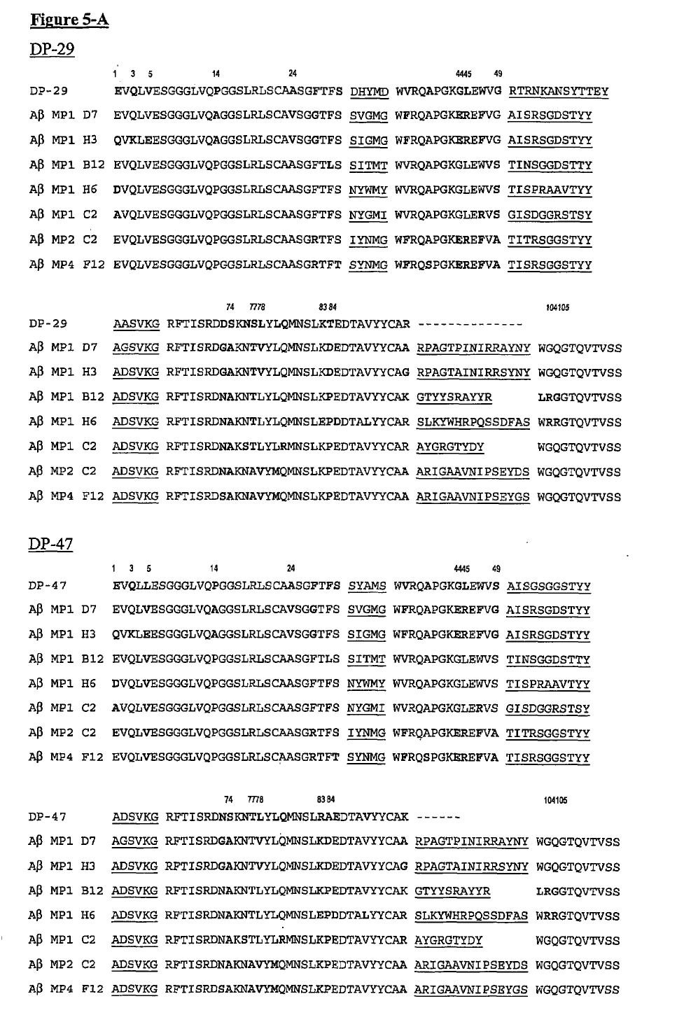

FIG. 4: Object recognition index of female APP transgenic mice (B,D,C) which were vehicle-treated (C), nanobody treated (B, D) as compared to female non-transgenic controls (F1). All mice were age-matched.

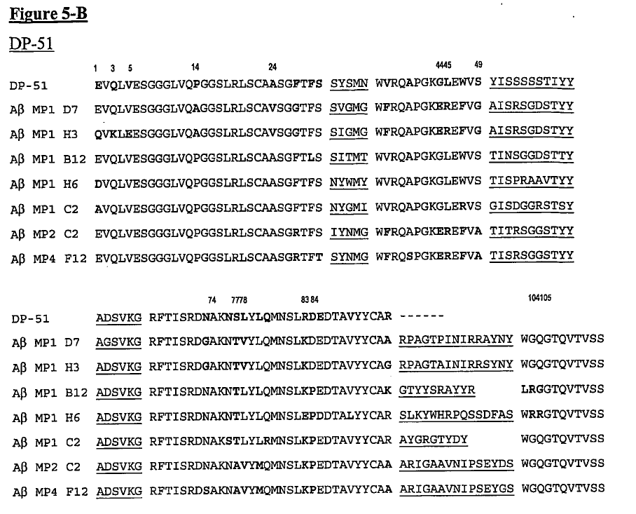

FIGS. 5A-B: Sequence alignment of some of the Nanobodies of the invention and human VH3 germline sequences DP-29, DP-47 and DP-51

### EXPERIMENTAL PART

### Example 1

### Antigen Specific Nanobodies

The sequences represented by SEQ ID NOs: 73-84 (Table 3) are Nanobodies obtained from llamas immunized with aggregated synthetic peptides. To generate nanobodies synthetic peptides, Aβ40 (SEQ ID NO 187) and Aβ42 (SEQ ID NO 188), were used as immunogens. Llamas were injected with in vitro aggregated synthetic Aβ40 or Aβ42 preparations formulated in specol-adjuvant. Animals were immunized with six subcutaneous injections (100 μg/dose) at weekly intervals. One week after the last boost, sera were collected to define antibody titers against Aβ40 and Aβ42 by ELISA. In this ELISA, 96-well plates (Maxisorp; Nunc) were coated with peptides following the protocol as described by Bohrmann et al (1999) J. Biol. Chem. 247, 15990-15995. After blocking and adding diluted sera samples, the presence of anti-A-beta nanobodies was demonstrated by using rabbit anti-llama immunoglobulin antiserum and anti-rabbit immunoglobulin alkaline phosphatase conjugate. The titer exceeded 12800 for the three animals.

The nanobodies were produced in E. Coli as soluble periplasmic proteins, harboring at their carboxy terminus a hexahistidine tag and a myc-tag. The presence of the hexahistidine tag is useful for one-step purification by IMAC chromatography. The myc-tag enables easy detection by immunological methods. The binding of the recombinant proteins represented by SEQ ID NOs: 73-84 and 85-105 to the synthetic peptides was demonstrated by ELISA. In this ELISA 96-well plates were coated with the peptides as described above. After blocking the plates with 2% casein, either crude periplasmic extracts or purified nanobodies were added to individual wells at several dilutions. After incubation for 1 hour, the wells were washed and subsequently a mouse anti-myc monoclonal antibody and a rabbit anti-mouse-alkaline phosphatase conjugate (Sigma A 1902) were used to detect the bound nanobodies.

In FIGS. 1a and 1b the ELISA signals obtained for 4 dilutions of the periplasmic extracts (nanobodies listed in Table 3) on both AB-40 or A13-42 peptides were plotted. For all clones even at 1/625 dilution of the extracts specific binding was demonstrated. No signal was present when periplasmic extracts were tested at ⅕ dilution, on plates where no antigen was coated. The proteins were also purified by IMAC chromatography and tested by ELISA on Aα40 and Aβ42 peptides. The protein concentration of the nanobodies after purification was determined spectrophotometrically at 280 nm by using their calculated molecular weight and extinction coefficient. As shown in FIG. 2, this ELISA experiment demonstrates that the nanobodies listed in Table 3 recognize solid phase coated Aβ40 and Aβ42 peptides equally well.

### Example 2

### Nanobodies Specific for Aggregated A-Beta Peptides Recognize Amyloid Plaque

Nanobodies directed against A-beta peptides are useful as probes to detect amyloid plaques in histological slices through APP transgenic mouse brain. These APP transgenic mice express human APP, accumulate Aβ40 and Aβ42 peptides in brain, display brain amyloid plaques highly similar to diffuse and senile plaques in human AD patient brains, show a memory deficit and other characteristics of the amyloid pathology of human AD (described in Moechars et al., (1999) J. Biol. Chem. 274, 6483-6492). Brains of amyloid plaque-containing mice are fixed, cut in 40 μM slices and the anti-A-beta nanobody is used as a primary probe, in combination with e.g. peroxidase. In this way we have been able to stain the plaques with labeled secondary antibody to stain amyloid plaques. As can be observed in FIG. 3 amyloid plaques are specifically recognized by the nanobodies.

### Example 3

### Nanobodies Specific for Aggregated A-Beta are Efficient for Treatment

Anti-A-beta nanobodies are injected intraperitoneally (50 μg/animal) in transgenic APP mice, whereas a control group of APP transgenic mice is vehicle-only treated. Injections are given during three consecutive days. On day 2 and 3 an object recognition test was carried out. In this test mice were familiarized for one hour to a Plexiglas open-field box (52×52×40 cm) with black vertical walls and a translucent floor, dimly illuminated by a lamp placed underneath the box. The next day the animals were placed in the same box and submitted to a 10 minutes acquisition trial. During this trial mice were placed individually in the open field in the presence of object A (blue ball or red cube, similar sized of ca. 4 cm), and the frequency of exploring object A (when the animals snout was directed towards the object at a distance of <1 cm and the mice were actively sniffing in the direction of the object) was recorded (Freq _{AA}). During a 10 minutes retention trial (second trial) which was performed 3 hours later, a novel object (object B, red cube or blue ball) was placed together with the familiar object (object A) into the open field. The frequency with which the animal explored the two objects was recorded (Freq _{A} and Freq _{B}).

The recognition index (RI) defined as the ratio of the frequency in which the novel object was explored over the frequency in which both objects were explored [Freq _{B}/(Freq _{A}+Freq _{B})×100] was used to measure non-spatial memory.

As can been seen in FIG. 4, mice treated with anti-A-beta nanobodies show an increased recognition index.

The results from FIG. 3 and FIG. 4, together with the observation by Hock et al ((2003) Neuron, 38, 547-554) that beneficial clinical effects are observed in patients expressing antibodies able to recognize amyloid plaques in transgenic mouse brain slices, indicate a therapeutic potential for the anti-A-beta nanobodies described in this invention.

### Example 4

### Modulation of the Pharmocokinetics

In order to prolong the serum half-life of nanobodies upon intravenous or intra-peritoneal administration bispecific molecules antibodies were constructed. Examples of such molecules are given in Table 8. In these polypeptides one or more A-beta specific nanobodies is genetically linked to nanobodies specific for serum albumin such as MSA21 and HSA MP13 B11. As a non limiting example of a suitable linker sequence, three alanines were used in this example.

### Example 5

### Humanization of A-Beta MP1 B12

### 1) Homology Between Anti-A-Beta Sequences and Human Germline Heavy Chain V-Region DP-29, DP-47 and DP-51

Alignment of some of the Nanobodies of the invention and human VH3 germline sequences DP-29, DP-47 and DP-51 revealed that AA changes may be performed at the following positions:
AA changes in FR1 on position 1, 3, 5, 14 and 24
AA changes in FR2 on position 44, 45 and 49
AA changes in FR3 on position 74, 77, 78, 83 and 84
AA change in FR4 (derived from the germline J segments) on position 104 and 105

### 2) Mutagenesis of Aβ MP1 B12

AP MP1 B12 (SEQ ID NO: 77) was mutated by using site-directed mutagenesis method as described by Chen and Ruffner (Nucleic Acids Research, 1998). Plasmid DNA was used as template in combination with 2 mutagenic primers introducing the desired mutation(s). The 2 primers are each complementary to opposite strands of the template plasmid DNA. In a polymerase reaction using the Pfu DNA polymerase each strand is extended from the primer sequence during a cycling program using a limited number of cycles. This results in a mixture of wild type and mutated strands. Digestion with DpnI results in selection of the mutated in vitro synthesized DNA strand, since only the template strand is sensitive for digestion. The DNA was precipitated and transformed into XL-Gold ultracompetent cells and analyzed for the required mutation by sequence analysis.

Plasmid was prepared from mutant clones in XL-Gold ultracompetent cells and was transformed into WK-6 electrocompetent cells. Overnight culture was started by inoculating a single colony in LB containing 2% glucose and 100 μg/ml ampicillin. This overnight culture was diluted 100-fold in 300 ml TB medium containing 100 μg/ml ampicillin, and incubated at 37° C. until OD600 nm=2, when 1 mM IPTG (final concentration) was added and the culture was incubated for 3 more hours at 37° C. or overnight at 28° C. Cultures were centrifuged for 15 minutes at 4,500 rpm. The pellet was frozen overnight or for 1 hour at −20° C. Next, the pellet was thawed at room temperature for 40 minutes, re-suspended in 15 ml peri buffer (50 mM NaHPO₄, 300 mM NaCl) and shaken for 1 hour. Periplasmic fraction was isolated by centrifugation for 20 minutes at 14000 rpm. The supernatant containing the nanobody was loaded on TALON (ClonTech) and purified to homogeneity. The yield of nanobody was determined using the calculated extinction coefficient.

All mutant nanobodies expressed comparably to the wild type. The mutants were analyzed for their binding activity in an in vitro binding assay as described in Example 1.

### Example 5

The Nanobodies and polypeptides of the invention are tested in two in vivo animal tests, the Novel Object Recognition Test and the Morris Water Maze test:

a) Novel Object Recognition Test

The protocol that is used follows the method described by Dewachter I. et al (Journal of Neuroscience, 2002, 22(9):3445-3453). Mice are familiarized for one hour to a Plexiglas open-field box (52×52×40 cm) with black vertical walls and a translucent floor, dimly illuminated by a lamp placed underneath the box. The next day the animals are placed in the same box and submitted to a 10 minutes acquisition trial. During this trial mice are placed individually in the open field in the presence of 2× object A (blue ball or red cube, similar sized of 4 cm), and the duration (timeAA) and the frequency (FreqAA) exploring object A (when the animals snout is directed towards the object at a distance of <1 cm and the mice are actively sniffing in the direction of the object) is recorded by a computerized system (Ethovision, Noldus information Technology, Wageningen, the Netherlands). During a 10 minutes retention trial (second trial) performed 3 hours later, a novel object (object B, red cube or blue ball) is placed together with the familiar object (object A) into the open field. (Freq A and Freq Band TimeA and TimeB, respectively). The recognition index (RI), defined as the ratio of the duration in which the novel object is explored over the duration in which both objects are explored [Time B/(Time A+Time B)×100], is used to measure non-spatial memory. The duration and frequency object A is explored during the acquisition trial (TimeAA and FreqAA) is used to measure curiosity.

Mice that do not distinguish between an old object and a new one, have a recognition index of 50. Mice that recognize the old object, will preferably explore the novel object and hence the recognition index becomes >50. Mice that exclusively explore the novel object have a recognition index of 100.

In this test, wild-type mice treated with PBS as a control showed a recognition index of 66.4+/−3.2 (all values mentioned are an average for 10 mice); untreated APP mice showed a recognition index of 50.7+/−3.8, and APP mice treated with a Nanobody construct based on the H6 A-Beta Nanobody [SEQ ID NO: 76] linked at the C-terminus to the blood brain barrier crossing Nanobody FC44 [SEQ ID NO: 189] via a linker sequence GGGGSGAGGA [SEQ ID NO:191] showed a recognition index of 62.0+/−2.4.

b) Morris Water Maze Test

The pool (a white, circular vessel 1 m in diameter) contains water at 20° C. with titanium dioxide as an odorless, nontoxic additive to hide the escape platform (1 cm beneath the water level). Swimming of each mouse is videotaped and analyzed (Ethovision, Noldus information Technology, Wageningen, the Netherlands). Prior to training, each mouse is placed on top of the platform for 15 seconds. For place navigation tests, mice are trained to locate the hidden platform in five blocks of three trials over three consecutive days. Each trial consists of a forced swim test of maximum 120 seconds, followed by 60 seconds of rest. The time each mouse needed for location of the platform is measured. The five consecutive trials result in a learning curve. 24 hours after the last training, each animal has a probe trial with the platform removed. Mice are allowed to search for 60 seconds and quadrant search time and crossings of the original platform position is measured. Mice that refuse to swim and search the platform, but instead wait until the performer takes them out of the pool, the so-called “floaters”, are excluded from analysis. During the final probe test, mice are allowed to search the previous location of the platform for 60 seconds after the platform is removed.

The results of this test are as summarized in Table 13 below. The Nanobody construct used was a H6 A-Beta Nanobody [SEQ ID NO: 76] linked at the C-terminus to the blood brain barrier crossing Nanobody FC44 [SEQ ID NO: 189] via a linker sequence GGGGSGAGGA [SEQ ID NO:191]

### Tables

TABLE 1
SEQ ID NO's 1-36

<Name, SEQ ID #; PRT (protein); ->
Sequence
<FR1, SEQ ID NO:1 ;PRT;->
QVQLQESGGGXVQAGGSLRLSCAASG
<FR2, SEQ ID NO:2 ;PRT;->
WXRQAPGKXXEXVA
<FR3, SEQ ID NO:3 ;PRT;->
RFTISRDNAKNTVYLQMNSLXXEDTAVYYCAA
<FR4, SEQ ID NO:4 ;PRT;->
XXQGTXVTVSS
<FR1, SEQ ID NO:5 ;PRT;->
QVQLQESGGGLVQAGGSLRLSCAASG
<FR2, SEQ ID NO:6 ;PRT;->
WFRQAPGKERELVA
<FR2, SEQ ID NO:7 ;PRT;->
WFRQAPGKEREFVA
<FR2, SEQ ID NO:8 ;PRT;->
WFRQAPGKEREGA
<FR2, SEQ ID NO:9 ;PRT;->
WFRQAPGKQRELVA
<FR2, SEQ ID NO:10 ;PRT;->
WFRQAPGKQREFVA
<FR2, SEQ ID NO:11 ;PRT;->
WYRQAPGKGLEWA
<FR3, SEQ ID NO:12 ;PRT;->
RFTISRDNAKNTVYLQMNSLKPEDTAVYYCAA
<FR4, SEQ ID NO:13 ;PRT;->
WGQGTQVTVSS
<FR4, SEQ ID NO:14 ;PRT;->
WGQGTLVTVSS
<CDR1, SEQ ID NO:15 ;PRT;->
SFGMS
<CDR1, SEQ ID NO:16 ;PRT;->
LNLMG
<CDR1, SEQ ID NO:17 ;PRT;->
INLLG
<CDR1, SEQ ID NO:18 ;PRT;->
NYWMY
<CDR2, SEQ ID NO:19 ;PRT;->
SISGSGSDTLYADSVKG
<CDR2, SEQ ID NO:20 ;PRT;->
TITVGDSTNYADSVKG
<CDR2, SEQ ID NO:21 ;PRT;->
TITVGDSTSYADSVKG
<CDR2, SEQ ID NO:22 ;PRT;->
SINGRGDDTRYADSVKG
<CDR2, SEQ ID NO:23 ;PRT;->
AISADSSTKNYADSVKG
<CDR2, SEQ ID NO:24 ;PRT;->
AISADSSDKRYADSVKG
<CDR2, SEQ ID NO:25 ;PRT;->
RISTGGGYSYYADSVKG
<CDR3, SEQ ID NO:26 ;PRT;->
DREAQVDTLDFDY
<CDR3, SEQ ID NO:27 ;PRT;->
GGSLSR
<CDR3, SEQ ID NO:28 ;PRT;->
RRTWHSEL
<CDR3, SEQ ID NO:29 ;PRT;->
GRSVSRS
<CDR3, SEQ ID NO:30 ;PRT;->
GRGSP
<Myc-tag, SEQ ID NO:31 ;PRT;->
AAAEQKLISEEDLNGAA
<GS30, SEQ ID NO:32 ;PRT;->
GGGGSGGGGSGGGGSGGGGSGGGGSGGGGS
<GS33, SEQ ID NO:33 ;PRT;->
GGGGSGGGS
<ALB-1, SEQ ID NO:34 ;PRT;->
AVQLVESGGGLVQPGNSLRLSCAASGFTFRSFGMSWVRQAPGKEPEWVSS
ISGSGSDTLYADSVKGRFTISRDNAKTTLYLQMNSLKPEDTAVYYCTIGG
SLSRSSQGTQVTVSS
<ALB-8, SEQ ID NO:35 ;PRT;->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSS
<ALB-2, SEQ ID NO:36 ;PRT;->
AVQLVESGGGLVQGGGSLRLACAASERIFDLNLMGWYRQGPGNERELVAT
CITVGDSTNYADSVKGRFTISMDYTKQTVYLHMNSLRPEDTGLYYCKIRR
TWHSELWGQGTQVTVSS

TABLE 2
SEQ ID NO's 37-72

<Name, SEQ ID #; PRT (protein); ->
Sequence
<CDR1, SEQ ID NO:37 ;PRT;->
GGTFSSVGMG
<CDR1, SEQ ID NO:38 ;PRT;->
GFTFSNYGMI
<CDR1, SEQ ID NO:39 ;PRT;->
GGTFSSIGMG
<CDR1, SEQ ID NO:40 ;PRT;->
GFTFSNYWMY
<CDR1, SEQ ID NO:41 ;PRT;->
GFTLSSITMT
<CDR1, SEQ ID NO:42 ;PRT;->
GRTFSIYNMG
<CDR1, SEQ ID NO:43 ;PRT;->
GRTFTSYNMG
<CDR1, SEQ ID NO:44 ;PRT;->
GFTFSNYWMY
<CDR1, SEQ ID NO:45 ;PRT;->
GGTFSSIGMG
<CDR1, SEQ ID NO:46 ;PRT;->
GGIYRVNTVN
<CDR1, SEQ ID NO:47 ;PRT;->
GFTFSNYWMY
<CDR1, SEQ ID NO:48 ;PRT;->
GFTLSSITMT
<CDR2, SEQ ID NO:49 ;PRT;->
AISRSGDSTYYAGSVKG
<CDR2, SEQ ID NO:50 ;PRT;->
GISDGGRSTSYADSVKG
<CDR3, SEQ ID NO:51 ;PRT;->
AISRSGDSTYYADSVKG
<CDR3, SEQ ID NO:52 ;PRT;->
TISPRAAVTYYADSVKG
<CDR3, SEQ ID NO:53 ;PRT;->
TINSGGDSTTYADSVKG
<CDR3, SEQ ID NO:54 ;PRT;->
TITRSGGSTYYADSVKG
<CDR2, SEQ ID NO:55 ;PRT;->
TISRSGGSTYYADSVKG
<CDR2, SEQ ID NO:56 ;PRT;->
TISPRAGSTYYADSVKG
<CDR2, SEQ ID NO:57 ;PRT;->
AISRSGDSTYYADSVKG
<CDR2, SEQ ID NO:58 ;PRT;->
TITRAGSTNYVESVKG
<CDR2, SEQ ID NO:59 ;PRT;->
TISPRAANTYYADSVKG
<CDR2, SEQ ID NO:60 ;PRT;->
TINSGGDSTTYADSVKG
<CDR3, SEQ ID NO:61 ;PRT;->
RPAGTPINIRRAYNY
<CDR3, SEQ ID NO:62 ;PRT;->
AYGRGTYDY
<CDR3, SEQ ID NO:63 ;PRT;->
RPAGTAINIRRSYNY
<CDR3, SEQ ID NO:64 ;PRT;->
SLKYWHRPQSSDFAS
<CDR3, SEQ ID NO:65 ;PRT;->
GTYYSRAYYR
<CDR3, SEQ ID NO:66 ;PRT;->
ARIGAAVNIPSEYDS
<CDR3, SEQ ID NO:67 ;PRT;->
RPAGTPINIRRAYNY
<CDR3, SEQ ID NO:68 ;PRT;->
SLIYKARPQSSDFVS
<CDR3, SEQ ID NO:69 ;PRT;->
RPAGTAINIRRSYNY
<CDR3, SEQ ID NO:70 ;PRT;->
NGRWRSWSSQRDY
<CDR3, SEQ ID NO:71 ;PRT;->
SLRYRDRPQSSDFLF
<CDR3, SEQ ID NO:72 ;PRT;->
GTYYSRAYYR

TABLE 3

Sequence listing of nanobodies directed
against A-beta

<Name, SEQ ID #; PRT (protein); ->
Sequence
<A-BETA MP1 D7, SEQ ID NO:73 ;PRT;->
EVQLVESGGGLVQAGGSLRLSCAVSGGTFSSVGMGWFRQAPGKEREFVGA
ISRSGDSTYYAGSVKGRFTISRDGAKNTVYLQMNSLKDEDTAVYYCAARP
AGTPINIRRAYNYWGQGTQVTVSS
<A-BETA MP1 C2, SEQ ID NO:74 ;PRT;->
AVQLVESGGGLVQPGGSLRLSCAASGFTFSNYGMIWVRQAPGKGLERVSG
ISDGGRSTSYADSVKGRFTISRDNAKSTLYLRMNSLKPEDTAVYYCARAY
GRGTYDYWGQGTQVTVSS
<A-BETA MP1 H3, SEQ ID NO:75 ;PRT;->
QVKLEESGGGLVQAGGSLRLSCAVSGGTFSSIGMGWFRQAPGKEREFVGA
ISRSGDSTYYADSVKGRFTISRDGAKNTVYLQMNSLKDEDTAVYYCAGRP
AGTAINIRRSYNYWGQGTQVTVSS
<A-BETA MP1 H6, SEQ ID NO:76 ;PRT;->
DVQLVESGGGLVQPGGSLRLSCAASGFTFSNYWMYWVRQAPGKGLEWVST
ISPRAAVTYYADSVKGRFTISRDNAKNTLYLQMNSLEPDDTALYYCARSL
KYWHRPQSSDFASWRRGTQVTVSS
<A-BETA MP1 B12, SEQ ID NO:77 ;PRT;->
EVQLVESGGGLVQPGGSLRLSCAASGFTLSSITMTWVRQAPGKGLEWVST
INSGGDSTTYADSVKGRFTISRDNAKNTLYLQMNSLKPEDTAVYYCAKGT
YYSRAYYRLRGGTQVTVSS
<A-BETA MP2 C2, SEQ ID NO:78 ;PRT;->
EVQLVESGGGLVQPGGSLRLSCAASGRTFSIYNMGWFRQAPGKEREFVAT
ITRSGGSTYYADSVKGRFTISRDNAKNAVYMQMNSLKPEDTAVYYCAAAR
IGAAVNIPSEYDSWGQGTQVTVSS
<A-BETA MP4 F12, SEQ ID NO:79 ;PRT;->
EVQLVESGGGLVQPGGSLRLSCAASGRTFTSYNMGWFRQSPGKEREFVAT
ISRSGGSTYYADSVKGRFTISRDSAKNAVYMQMNSLKPEDTAVYYCAAAR
IGAAVNIPSEYGSWGQGTQVTVSS
<A-BETA PMP2 C7, SEQ ID NO:80 ;PRT;->
QVKLEESGGGLVQPGGSLRLSCAASGFTFSNYWMYWVRQAPGKGLEWVST
ISPRAGSTYYADSVKGRFTISRDNAKNTLYLQMNSLEPDDTALYYCARSL
IYKARPQSSDFVSWRQGTQVTVSS
<A-BETA PMP2 D2, SEQ ID NO:81 ;PRT;->
AVQLVDSGGGLVQAGGSLRLSCAVSGGTFSSIGMGWFRQAPGKEREFVGA
ISRSGDSTYYADSVKGRFTISRDGAKNTVYLQMNSLKDEDTAVYYCAGRP
AGTAINIRRSYNYWGQGTQVTVSS
<A-BETA PMP2 E10, SEQ ID NO:82 ;PRT;->
AVQLVESGGGLVQPGGSLRLSCAASGGIYRVNTVNWYRQAPGLQRELVAT
ITRAGSTNYVESVKGRFTISLDNAKNTMYLQMNSLKPDDTGVYYCNVNGR
WRSWSSQRDYWGQGTQVTVSS
<A-BETA PMP2 G6, SEQ ID NO:83 ;PRT;->
QVKLEESGGGLVQPGGSLRLSCAASGFTFSNYWMYWVRQAPGKGLEWVST
ISPRAANTYYADSVKGRFTISRDNAKNTLYLQMNSLEPDDTALYYCAKSL
RYRDRPQSSDFLFWRQGTQVTVSS
<A-BETA PMP2 D6, SEQ ID NO:84 ;PRT;->
AVQLVESGGGLVQPGGSLRLSCAASGFTLSSITMTWVRQAPGKGLEWVST
INSGGDSTTYADSVKGRFTISRDNAKNTLYLQMNSLKPEDTAVYYCAKGT
YYSRAYYRLRGGTQVTVSS

TABLE 4

Sequence listing of some non-limiting examples
of humanized nanobodies directed against A-beta

<Name, SEQ ID #; PRT (protein); ->
Sequence
<A-BETA MP1 D7-1, SEQ ID NO:85 ;PRT;->
EVQLVESGGGLVQPGGSLRLSCAVSGGTFSSVGMGWFRQAPGKEREFVGA
ISRSGDSTYYAGSVKGRFTISRDGAKNTVYLQMNSLKDEDTAVYYCAARP
AGTPINIRRAYNYWGQGTQVTVSS
<A-BETA MP1 D7-2, SEQ ID NO:86 ;PRT;->
EVQLVESGGGLVQPGGSLRLSCAVSGGTFSSVGMGWFRQAPGKELEFVGA
ISRSGDSTYYAGSVKGRFTISRDGAKNTVYLQMNSLKDEDTAVYYCAARP
AGTPINIRRAYNYWGQGTQVTVSS
<A-BETA MP1 D7-3, SEQ ID NO:87 ;PRT;->
EVQLVESGGGLVQPGGSLRLSCAVSGGTFSSVGMGWFRQAPGKGLEFVGA
ISRSGDSTYYAGSVKGRFTISRDGAKNTVYLQMNSLKDEDTAVYYCAARP
AGTPINIRRAYNYWGQGTQVTVSS
<A-BETA MP1 D7-4, SEQ ID NO:88 ;PRT;->
EVQLVESGGGLVQPGGSLRLSCAVSGGTFSSVGMGWFRQAPGKGLEFVGA
ISRSGDSTYYAGSVKGRFTISRDGAKNTLYLQMNSLKDEDTAVYYCAARP
AGTPINIRRAYNYWGQGTQVTVSS
<A-BETA MP1 D7-5, SEQ ID NO:89 ;PRT;->
EVQLVESGGGLVQPGGSLRLSCAVSGGTFSSVGMGWFRQAPGKGLEFVGA
ISRSGDSTYYAGSVKGRFTISRDGAKNTLYLQMNSLKTEDTAVYYCAARP
AGTPINIRRAYNYWGQGTQVTVSS
<A-BETA MP1 D7-6, SEQ ID NO:90 ;PRT;->
EVQLVESGGGLVQPGGSLRLSCAVSGGTFSSVGMGWFRQAPGKGLEFVGA
ISRSGDSTYYAGSVKGRFTISRDGAKNSLYLQMNSLKTEDTAVYYCAARP
AGTPINIRRAYNYWGQGTQVTVSS
<A-BETA MP1 D7-7, SEQ ID NO:91 ;PRT;->
EVQLVESGGGLVQPGGSLRLSCAVSGGTFSSVGMGWFRQAPGKGLEFVGA
ISRSGDSTYYAGSVKGRFTISRDGSKNSLYLQMNSLKTEDTAVYYCAARP
AGTPINIRRAYNYWGQGTQVTVSS
<A-BETA MP1 H3-1, SEQ ID NO:92 ;PRT;->
EVKLEESGGGLVQAGGSLRLSCAVSGGTFSSIGMGWFRQAPGKEREFVGA
ISRSGDSTYYADSVKGRFTISRDGAKNTVYLQMNSLKDEDTAVYYCAGRP
AGTAINIRRSYNYWGQGTQVTVSS
<A-BETA MP1 H3-2, SEQ ID NO:93 ;PRT;->
EVQLEESGGGLVQAGGSLRLSCAVSGGTFSSIGMGWFRQAPGKEREFVGA
ISRSGDSTYYADSVKGRFTISRDGAKNTVYLQMNSLKDEDTAVYYCAGRP
AGTAINIRRSYNYWGQGTQVTVSS
<A-BETA MP1 H3-3, SEQ ID NO:94 ;PRT;->
EVQLVESGGGLVQAGGSLRLSCAVSGGTFSSIGMGWFRQAPGKEREFVGA
ISRSGDSTYYADSVKGRFTISRDGAKNTVYLQMNSLKDEDTAVYYCAGRP
AGTAINIRRSYNYWGQGTQVTVSS
<A-BETA MP1 H3-4, SEQ ID NO:95 ;PRT;->
EVQLVESGGGLVQPGGSLRLSCAVSGGTFSSIGMGWFRQAPGKEREFVGA
ISRSGDSTYYADSVKGRFTISRDGAKNTVYLQMNSLKDEDTAVYYCAGRP
AGTAINIRRSYNYWGQGTQVTVSS
<A-BETA MP1 H3-5, SEQ ID NO:96 ;PRT;->
EVQLVESGGGLVQPGGSLRLSCAVSGGTFSSIGMGWFRQAPGKGREFVGA
ISRSGDSTYYADSVKGRFTISRDGAKNTVYLQMNSLKDEDTAVYYCAGRP
AGTAINIRRSYNYWGQGTQVTVSS
<A-BETA MP1 H3-6, SEQ ID NO:97 ;PRT;->
EVQLVESGGGLVQPGGSLRLSCAVSGGTFSSIGMGWFRQAPGKGLEFVGA
ISRSGDSTYYADSVKGRFTISRDGAKNTVYLQMNSLKDEDTAVYYCAGRP
AGTAINIRRSYNYWGQGTQVTVSS
<A-BETA MP1 H3-7, SEQ ID NO:98 ;PRT;->
EVQLVESGGGLVQPGGSLRLSCAVSGGTFSSIGMGWFRQAPGKGLEFVGA
ISRSGDSTYYADSVKGRFTISRDGSKNTVYLQMNSLKDEDTAVYYCAGRP
AGTAINIRRSYNYWGQGTQVTVSS
<A-BETA MP1 H3-8, SEQ ID NO:99 ;PRT;->
EVQLVESGGGLVQPGGSLRLSCAVSGGTFSSIGMGWFRQAPGKGLEFVGA
ISRSGDSTYYADSVKGRFTISRDGSKNSVYLQMNSLKDEDTAVYYCAGRP
AGTAINIRRSYNYWGQGTQVTVSS
<A-BETA MP1 H3-9, SEQ ID NO:100 ;PRT;->
EVQLVESGGGLVQPGGSLRLSCAVSGGTFSSIGMGWFRQAPGKGLEFVGA
ISRSGDSTYYADSVKGRFTISRDGSKNSLYLQMNSLKDEDTAVYYCAGRP
AGTAINIRRSYNYWGQGTQVTVSS
<A-BETA MP1 H3-10, SEQ ID NO:101 ;PRT;->
EVQLVESGGGLVQPGGSLRLSCAVSGGTFSSIGMGWFRQAPGKGLEFVGA
ISRSGDSTYYADSVKGRFTISRDGSKNSLYLQMNSLKTEDTAVYYCAGRP
AGTAINIRRSYNYWGQGTQVTVSS
<A-BETA MP1 H6-1, SEQ ID NO:102 ;PRT;->
EVQLVESGGGLVQPGGSLRLSCAASGFTFSNYWMYWVRQAPGKGLEWVST
ISPRAAVTYYADSVKGRFTISRDNAKNTLYLQMNSLEPDDTALYYCARSL
KYWHRPQSSDFASWRRGTQVTVSS
<A-BETA MP1 H6-2, SEQ ID NO:103 ;PRT;->
EVQLVESGGGLVQPGGSLRLSCAASGFTFSNYWMYWVRQAPGKGLEWVST
ISPRAAVTYYADSVKGRFTISRDNSKNTLYLQMNSLEPDDTALYYCARSL
KYWHRPQSSDFASWRRGTQVTVSS
<A-BETA MP1 H6-3, SEQ ID NO:104 ;PRT;->
EVQLVESGGGLVQPGGSLRLSCAASGFTFSNYWMYWVRQAPGKGLEWVST
ISPRAAVTYYADSVKGRFTISRDNSKNSLYLQMNSLEPDDTALYYCARSL
KYWHRPQSSDFASWRRGTQVTVSS
<A-BETA MP1 H6-4, SEQ ID NO:105 ;PRT;->
EVQLVESGGGLVQPGGSLRLSCAASGFTFSNYWMYWVRQAPGKGLEWVST
ISPRAAVTYYADSVKGRFTISRDNSKNSLYLQMNSLETDDTALYYCARSL
KYWHRPQSSDFASWRRGTQVTVSS

TABLE 5

Sequence listing of anti-mouse serum
albumin nanobodies

<Name, SEQ ID #; PRT (protein); ->
Sequence
<MSA21, SEQ ID NO:106 ;PRT;->
QVQLQESGGGLVQPGGSLRLSCEASGFTFSRFGMTWVRQAPGKGVEWVSG
ISSLGDSTLYADSVKGRFTISRDNAKNTLYLQMNSLKPEDTAVYYCTIGG
SLNPGGQGTQVTVSS
<MSA24, SEQ ID NO:107 ;PRT;->
QVQLQESGGGLVQPGNSLRLSCAASGFTFRNFGMSWVRQAPGKEPEWVSS
ISGSGSNTIYADSVKDRFTISRDNAKSTLYLQMNSLKPEDTAVYYCTIGG
SLSRSSQGTQVTVSS
<MSA210, SEQ ID NO:108 ;PRT;->
QVQLQESGGGLVQPGGSLRLTCTASGFTFSSFGMSWVRQAPGKGLEWVSA
ISSDSGTKNYADSVKGRFTISRDNAKKMLFLQMNSLRPEDTAVYYCVIGR
GSPSSQGTQVTVSS
<MSA212, SEQ ID NO:109 ;PRT;->
QVQLQESGGGLVQPGGSLRLTCTASGFTFRSFGMSWVRQAPGKGLEWVSA
ISADGSDKRYADSVKGRFTISRDNGKKMLTLDMNSLKPEDTAVYYCVIGR
GSPASQGTQVTVSS

TABLE 6

Sequence listing of anti-human serum
albumin nanobodies

<Name, SEQ ID #; PRT (protein); ->
Sequence
<HSA MP13 B11, SEQ ID NO:110 ;PRT;->
QVQLVESGGGLVQAGGSLRLSCAASGRAFIAYAMGWFRQGPGKEREFVAA
ISSYSGTNTNYADSVRGRFTISRDNVENMVYLQMNNLKPEDTAVYYCAAD
RRVLTSTSPFWGQGTQVTVSS
<HSA MP13 F12, SEQ ID NO:111 ;PRT;->
EVQLVESGGGLVQAGGSLRLSCAASGRTFSSYPMGWFRQASGKEREFVAA
ISRSGGSTYYEDFVKGRFTISRDNAKNTVYLQMNSLKPEDTAVYYCNVGK
VWGQGTQVTVSS
<HSA MP13 H6, SEQ ID NO:112 ;PRT;->
QVKLEESGGGLVQAGGSLRLSCAASGRAFIAYAMGWFRQGPGKEREFVAA
ISSYSGTNTNYADSVRGRFTISRDNVENMVYLQMNNLKPEDTAVYYCAAD
RRVLTSTSPFWGQGTQVTVSS
<HSA MP13 D6, SEQ ID NO:113 ;PRT;->
QVKLEESGGGLVQAGDSLRLSCVASGRTFSRYAVGWFRQAPGKPREFVAA
ISRSGGSTYHEDSVRGRFTISRDNTGNTVYLQMNSLKPEDTAVYYCNVAT
YWGLGTQVTVSS
<HSA MP13 E1, SEQ ID NO:114 ;PRT;->
QVKLEESGGGLVQAGGSLRLSCAASGRTFDSYDMGWFRQAPGKERDFVAF
ISWTGGRTVYADSVKGRFTISRDNAKNTVYLQMNSLKPEDTALYYCAASK
GAWPLYSLSSRYDYWGQGTQVTVSS

TABLE 7
Sequence listing of humanized anti-human serum albumin nanobodies

<Name, SEQ ID #; PRT (protein); ->
Sequence
<HSA MP13 B11 - 7, SEQ ID NO: 115; PRT;->
EVQLLESGGGLVQPGGSLRLSCAASGRAFIAYAMGWFRQAPGKGLEFVSAISSYSGTNTNYADSVRGRFTIS
RDNSKNTLYLQMNSLRAEDTAVYYCAADRRVLTSTSPFWGQGTLVTVSS
<HSA MP13 F12 - 6, SEQ ID NO: 116; PRT;->
EVQLLESGGGLVQPGGSLRLSCAASGRTFSSYPMGWFRQAPGKGLEFVSAISRSGGSTYYEDFVKGRFTISR
DNSKNTLYLQMNSLRAEDTAVYYCNVGKVWGQGTLVTVSS

TABLE 8
Some non-limiting examples of multispecific Nanobodies against A-beta

<Name, SEQ ID #; PRT (protein); ->
Sequence
Bivalent bispecific polypeptides directed against A-beta and mouse serum
albumin (joined with a linker)
<A-BETA MP1 D7-3A-MSA21, SEQ ID NO: 117; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSAAAQVQLQESGGGLVQPGGS
LRLSCEASGFTFSREGMTWVRQAPGKGVEWVSGISSLGDSTLYADSVKGRFTISRDNAKNTLYLQMNSLKPE
DTAVYYCTIGGSLNPGGQGTQVTVSS
<A-BETA MP1 H3-3A-MSA21, SEQ ID NO: 118; PRT;->
QVKLEESGGGLVQAGGSLRLSCAVSGGTFSSIGMGWFRQAPGKEREFVGAISRSGDSTYYADSVKGRFTTSR
DGAKNTVYLQMNSLKDEDTAVYYCAGRPAGTAINTRRSYNYWGQGTQVTVSSAAAQVQLQESGGGLVQPGGS
LRLSCEASGFTFSRFGMTWVRQAPGKGVEWVSGISSLGDSTLYADSVKGRFTISRDNAKNTLYLQMNSLKPE
DTAVYYCTIGGSLNPGGQGTQVTVSS
<A-BETA MP1 B12-3A-MSA21, SEQ ID NO: 119; PRT;->
EVQLVESGGGLVQPGGSLRLSCAASGFTLSSITMTWVRQAPGKGLEWVSTINSGGDSTTYADSVKGRFTISR
DNAKNTLYLQMNSLKPEDTAVYYCAKGTYYSRAYYRLRGGTQVTVSSAAAQVQLQESGGGLVQPGGSLRLSC
EASGFTFSRFGMTWVRQAPGKGVEWVSGISSLGDSTLYADSVKGRFTISRDNAKNTLYLQMNSLKPEDTAVY
YCTTGGSLNPGGQGTQVTVSS
<A-BETA MP1 H6-3A-MSA21, SEQ ID NO: 120; PRT;->
DVQLVESGGGLVQPGGSLRLSCAASGFTFSNYWMYWVRQAPGKGLEWVSTISPRAAVTYYADSVKGRFTTSR
DNAKNTLYLQMNSLEPDDTALYYCARSLKYWBRPQSSDFASWRRGTQVTVSSAAAQVQLQESGGGLVQPGGS
LRLSCEASGFTFSRFGMTWVRQAPGKGVEWVSGISSLGDSTLYADSVKGRFTISRDNAKNTLYLQMNSLKPE
DTAVYYCTTGGSLNPGGQGTQVTVSS
<A-BETA MP1 C2-3A-MSA21, SEQ ID NO: 121; PRT;->
AVQLVESGGGLVQPGGSLRLSCAASGFTFSNYGMIWVRQAPGKGLERVSGISDGGRSTSYADSVKGRFTISR
DNAKSTLYLRMNSLKPEDTAVYYCARAYGRGTYDYWGQGTQVTVSSAAAQVQLQESGGGLVQPGGSLRLSCE
ASGFTFSPFGMTWVRQAPGKGVEWVSGISSLGDSTLYADSVKGRFTISRDNAKNTLYLQMNSLKPEDTAVYY
CTIGGSLNPGGQGTQVTVSS
<A-BETA MP2 C2-3A-MSA21, SEQ ID NO: 122; PRT;->
EVQLVESGGGLVQPGGSLRLSCAASGRTFSIYNMGWFRQAPGKEREFVATITRSGGSTYYADSVKGRFTISR
DNAKNAVYMQMNSLKPEDTAVYYCAAARIGAAVNIPSEYDSWGQGTQVTVSSAAAQVQLQESGGGLVQPGGS
LRLSCEASGFTFSRFGMTWVRQAPGKGVEWVSGISSLGDSTLYADSVKGRFTISRDNAKNTLYLQMNSLKPE
DTAVYYCTIGGSLNPGGQGTQVTVSS
<A-BETA MP4 F12-3A-MSA21, SEQ ID NO: 123; PRT;->
EVQLVESGGGLVQPGGSLRLSCAASGRTFTSYNMGWFRQSPGKEREFVATISRSGGSTYYADSVKGRFTISR
DSAKNAVYMQMNSLKPEDTAVYYCAAARIGAAVNIPSEYGSWGQGTQVTVSSAAAQVQLQESGGGLVQPGGS
LRLSCEASGFTFSRFGMTWVRQAPGKGVEWVSGISSLGDSTLYADSVKGRFTISRDNAKNTLYLQMNSLKPE
DTAVYYCTIGGSLNPGGQGTQVTVSS
Bivalent bispecific polypeptides directed against A-beta and mouse serum
albumin (joined without a linker)
<A-BETA MP1 D7-MSA21, SEQ ID NO: 124; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSQVQLQESGGGLVQPGGSLRL
SCEASGFTFSRFGMTWVRQAPGKGVEWVSGISSLGDSTLYADSVKGRFTISRDNAKNTLYLQMNSLKPEDTA
VYYCTIGGSLNPGGQGTQVTVSS
<A-BETA MP1 H3-MSA21, SEQ ID MO: 125; PRT;->
QVKLEESGGGLVQAGGSLRLSCAVSGGTFSSIGMGWFRQAPGKEREFVGAISRSGDSTYYADSVKGRFTISR
DGAKNTVYLQMNSLKDEDTAVYYCAGRPAGTAINIRRSYNYWGQGTQVTVSSQVQLQESGGGLVQPGGSLRL
SCEASGFTFSRFGMTWVRQAPGKGVEWVSGTSSLGDSTLYADSVKGRFTISRDNAKNTLYLQMNSLKPEDTA
VYYCTTGGSLNPGGQGTQVTVSS
<A-BETA MP1 B12-MSA21, SEQ ID NO: 126; PRT;->
EVQLVESGGGLVQPGGSLRLSCAASGFTLSSITMTWVRQAPGKGLEWVSTINSGGDSTTYADSVKGRFTISR
DNAKNTLYLQMNSLKPEDTAVYYCAKGTYYSRAYYRLRGGTQVTVSSQVQLQESGGGLVQPGGSLRLSCEAS
GFTFSRFGMTWVRQAPGKGVEWVSGISSLGDSTLYADSVKGRFTISRDNAKNTLYLQMNSLKPEDTAVYYCT
TGGSLNPGGQGTQVTVSS
<A-BETA MP1 H6-MSA21, SEQ ID NO: 127; PRT;->
DVQLVESGGGLVQPGGSLRLSCAASGFTFSNYWMYWVRQAPGKGLEWVSTISPRAAVTYYADSVKGRFTISR
DNAKNTLYLQMNSLEPDDTALYYCARSLKYWHRPQSSDFASWRRGTQVTVSSQVQLQESGGGLVQPGGSLRL
SCEASGFTFSRFGMTWVRQAPGKGVEWVSGTSSLGDSTLYADSVKGRFTISRDNAKNTLYLQMMSLKPEDTA
VYYCTIGGSLNPGGQGTQVTVSS
<A-BETA MP1 C2-MSA21, SEQ ID NO: 128; PRT;->
AVQLVESGGGLVQPGGSLRLSCAASGFTFSNYGMIWVRQAPGKGLERVSGISDGGRSTSYADSVKGRFTTSR
DNAKSTLYLRMNSLKPEDTAVYYCARAYGRGTYDYWGQGTQVTVSSQVQLQESGGGLVQPGGSLRLSCEASG
FTFSRFGMTWVRQAPGKGVEWVSGISSLGDSTLYADSVKGRFTISRDNAKNTLYLQMNSLKPEDTAVYYCTI
GGSLNPGGQGTQVTVSS
<A-BETA MP2 C2-MSA21, SEQ ID NO: 129; PRT;->
EVQLVESGGGLVQPGGSLRLSCAASGRTFSIYNMGWFRQAPGKEREFVATITRSGGSTYYADSVKGRFTISR
DNAKNAVYMQMNSLKPEDTAVYYCAAARIGAAVNIPSEYDSWGQGTQVTVSSQVQLQESGGGLVQPGGSLRL
SCEASGFTFSRFGMTWVRQAPGKGVEWVSGISSLGDSTLYADSVKGRFTISRDNAKNTLYLQMNSLKPEDTA
VYYCTIGGSLNPGGQGTQVTVSS
<A-BETA MP4 F12-MSA21, SEQ ID NO: 130; PRT;->
EVQLVESGGGLVQPGGSLRLSCAASGRTFTSYMMGWFRQSPGKEREFVATISRSGGSTYYADSVKGRFTISR
DSAKNAVYMQMNSLKPEDTAVYYCAAARIGAAVNIPSEYGSWGQGTQVTVSSQVQLQESGGGLVQPGGSLRL
SCEASGFTFSRFGMTWVRQAPGKGVEWVSGISSLGDSTLYADSVKGRFTISRDNAKNTLYLQMNSLKPEDTA
VYYCTIGGSLNPGGQGTQVTVSS
Bivalent bispecific polypeptides comprising a nanobody directed against
A-beta and nanobody directed against human serum albumin (joined with a
linker)
<A-BETA PMP2 D2-ALB1, SEQ ID NO: 131; PRT;->
AVQLVDSGGGLVQAGGSLRLSCAVSGGTFSSIGMGWFRQAPGKEREFVGAISRSGDSTYYADSVKGRFTISR
DGAKNTVYLQMNSLKDEDTAVYYCAGRPAGTAINIRRSYNYWGQGTQVTVSSGGGGSGGGSAVQLVESGGGL
VQPGNSLRLSCAASGFTFRSFGMSWVRQAPGKEPEWVSSISGSGSDTLYADSVKGRFTISRDNAKTTLYLQM
NSLKPEDTAVYYCTIGGSLSRSSQGTQVTVSS
<ALB8- BA PMP2 D2, SEQ ID NO: 132; PRT;->
EVQLVESGGGLVQPGNSLRLSCAASGFTFSSFGMSWVRQAPGKGLEWVSSISGSGSDTLYADSVKGRFTISR
DNAKTTLYLQMNSLRPEDTAVYYCTIGGSLSRSSQGTLVTVSSGGGGSGGGSAVQLVDSGGGLVQAGGSLRL
SCAVSGGTFSSIGMGWFRQAPGKEREFVGAISRSGDSTYYADSVKGRFTISRDGAKNTVYLQMNSLKDEDTA
VYYCAGRPAGTAINIRRSYNYWGQGTQVTVSS
Bivalent bispecific polypeptides comprising humanized nanobody directed
against A-beta and humanized nanobody directed against human serum
albumin (joined with a linker)
<A-BETA MP1 -D7-3-3A- HSA MP13 B11 - 7, SEQ ID NO: 133; PRT;->
EVQLVESGGGLVQPGGSLRLSCAVSGGTFSSVGNGWFRQAPGKGLEFVGAISRSGESTYYAGSVKGRFTISR
DGAKNTVYLQMNSLKDEDTAVYYCAARPAGTPINIRRAYNYWGQGTQVTVSSAAAEVQLLESGGGLVQPGGS
LRLSCAASGRAFIAYANGWFRQAPGKGLEFVSAISSYSGTNTNYADSVRGRFTISRDNSKNTLYLQMNSLRA
EDTAVYYCAADRRVLTSTSPFWGQGTLVTVSS
<A-BETA MP1 -D7-5-3A- HSA MP13 B11 - 7, SEQ ID NO: 134; PRT;->
EVQLVESGGGLVQPGGSLRLSCAVSGGTFSSVGMGWFRQAPGKGLEFVGAISRSGDSTYYAGSVKGRFTISR
DGAKNTLYLQMNSLKTEDTAVYYCAARPAGTPINIRRAYNYWGQGTQVTVSSAAAEVQLLESGGGLVQPGGS
LRLSCAASGRAFIAYAMGWFRQAPGKGLEFVSAISSYSGTNTNYADSVRGRFTISRDNSKNTLYLQMNSLRA
EDTAVYYCAADRRVLTSTSPFWGQGTLVTVSS
<A-BETA MP1 -D7-7-3A- HSA MP13 B11 - 7, SEQ ID NO: 135; PRT;->
EVQLVESGGGLVQPGGSLRLSCAVSGGTFSSVGNGWFRQAPGKGLEFVGAISRSGDSTYYAGSVKGRFTISR
DGSKNSLYLQNNSLKTEDTAVYYCAARPAGTPINIRRAYNYWGQGTQVTVSSAAAEVQLLESGGGLVQPGGS
LRLSCAASGRAFIAYAMGWFRQAPGKGLEFVSATSSYSGTNTNYADSVRGRFTISRDNSKNTLYLQMNSLRA
EDTAVYYCAADRRVLTSTSPEWGQGTLVTVSS
<A-BETA MP1 H6-1-3A- HSA MP13 B11 - 7, SEQ ID NO: 136; PRT;->
EVQLVESGGGLVQPGGSLRLSCAASGFTFSNYWMYWVRQAPGKGLEWVSTISPRAAVTYYADSVKGRFTISR
DNAKNTLYLQMNSLEPDDTALYYCARSLKYWNRPQSSDFASWRRGTQVTVSSAAAEVQLLESGGGLVQPGGS
LRLSCAASGRAFIAYAMGWFRQAPGKGLEFVSAISSYSGTNTNYADSVRGRFTTSRDNSKNTLYLQMNSLRA
EDTAVYYCAADRRVLTSTSPFWGQGTLVTVSS
<A-BETA MP1 H6-2-3A- HSA MP13 B11 - 7, SEQ ID NO: 137; PRT;->
EVQLVESGGGLVQPGGSLRLSCAASGFTFSNYWMYWVRQAPGKGLEWVSTISPRAAVTYYADSVKGRFTISR
DNSKNTLYLQMNSLEPDDTALYYCARSLKYWHRPQSSDFASWRRGTQVTVSSAAAEVQLLESGGGLVQPGGS
LRLSCAASGRAFIAYAMGWFRQAPGKGLEFVSAISSYSGTNTNYADSVRGRFTISRDNSKNTLYLQMNSLRA
EDTAVYYCAADRRVLTSTSPFWGQGTLVTVSS
<A-BETA MP1 H6-3-3A- HSA MP13 B11 - 7, SEQ ID NO: 138; PRT;->
EVQLVESGGGLVQPGGSLRLSCAASGFTFSNYWMYWVRQAPGKGLEWVSTISPRAAVTYYADSVKGRFTISR
DNSKNSLYLQNNSLEPDDTALYYCARSLKYWNRPQSSDFASWRRGTQVTVSSAAAEVQLLESGGGLVQPGGS
LRLSCAASGRAFIAYAMGWFRQAPGKGbEFVSAISSYSGTNTNYADSVRGRFTISRDNSKNTLYLQMNSLRA
EDTAVYYCAADRRVLTSTSPFWGQGTLVTVSS
<A-BETA MP1 -H6-4-3A- HSA MP13 B11 - 7, SEQ ID NO: 139; PRT;->
EVQLVESGGGLVQPGGSLRLSCAASGFTFSNYWMYWVRQAPGKGLEWVSTISPRAAVTYYADSVKGRFTISR
DNSKNSLYLQMNSLETDDTALYYCARSLKYWBRPQSSDEASWRRGTQVTVSSAAAEVQLLESGGGLVQPGGS
LRLSCAASGRAFIAYAMGWFRQAPGKGLEFVSAISSYSGTNTNYADSVRGRFTISRDNSKNTLYLQMNSLRA
EDTAVYYCAADRRVLTSTSPFWGQGTLVTVSS
<A-BETA MP1 -H3-4-3A- HSA MP13 B11 - 7, SEQ ID NO: 140; PRT;->
EVQLVESGGGLVQPGGSLRLSCAVSGGTFSSIGMGWFRQAPGKEREFVGAISRSGDSTYYADSVKGRFTISR
DGAKNTVYLQMNSLKDEDTAVYYCAGRPAGTAINIRRSYNYWGQGTQVTVSSAAAEVQLLESGGGLVQPGGS
LRLSCAASGRAFIAYAMGWFRQAPGKGLEFVSAISSYSGTNTNYADSVRGRFTISRDNSKNTLYLQMNSLRA
EDTAVYYCAADRRVLTSTSPFWGQGTLVTVSS
<A-BETA MP4 -H3-10-3A- HSA MP13 B11 - 7, SEQ ID NO: 141; PRT;->
EVQLVESGGGLVQPGGSLRLSCAVSGGTFSSIGMGWFRQAPGKGLEFVGAISRSGDSTYYADSVKGRFTISR
DGSKNSLYLQMNSLKTEDTAVYYCAGRPAGTAINIRRSYNYWGQGTQVTVSSAAAEVQLLESGGGLVQPGGS
LRLSCAASGRAFIAYAMGWFRQAPGEGLEFVSAISSYSGTNTNYADSVRGRFTISRDNSKNTLYLQMNSLRA
EDTAVYYCAADRRVLTSTSPFWGQGTLVTVSS
Bivalent bispecific polypeptides comprising humanized nanobody directed
against A-beta and humanized nanobody directed against human serum
albumin (joined without a linker)
<A-BETA MP1 -D7-3- HSA MP13 B11 - 7, SEQ ID NO: 142; PRT;->
EVQLVESGGGLVQPGGSLRLSCAVSGGTFSSVGMGWFRQAPGKGLEFVGAISRSGDSTYYAGSVKGRFTISR
DGAKNTVYLQMNSLKDEDTAVYYCAARPAGTPINIRRAYNYWGQGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPTWGQGTLVTVSS
<A-BETA MP1 -D7-5- HSA MP13 B11 - 7, SEQ ID NO: 143; PRT;->
EVQLVESGGGLVQPGGSLRLSCAVSGGTFSSVGMGWFRQAPGKGLEFVGAISRSGDSTYYAGSVKGRFTISR
DGAKNTLYLQMNSLKTEDTAVYYCAARPAGTPINIRRAYNYWGQGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP1 -D7-7- HSA MP13 B11 - 7, SEQ ID NO: 143; PRT;->
EVQLVESGGGLVQPGGSLRLSCAVSGGTFSSVGMGWFRQAPGKGLEFVGAISRSGDSTYYAGSVKGRFTISR
DGSKNSLYLQMNSLKTEDTAVYYCAARPAGTPINIRRAYNYWGQGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP1 H6-1- HSA MP13 B11 - 7, SEQ ID NO: 144; PRT;->
EVQLVESGGGLVQPGGSLRLSCAASGFTFSNYWMYWVRQAPGKGLEWVSTISPRAAVTYYADSVKGRFTTSR
DNAKNTLYLQMNSLEPDDTALYYCARSLKYWBRPQSSDFASWRRGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP1 H6-2- HSA MP13 B11 - 7, SEQ ID NO: 145; PRT;->
EVQLVESGGGLVQPGGSLRLSCAASGFTFSNYWMYWVRQAPGKGLEWVSTISPRAAVTYYADSVKGRFTISR
DNSKNTLYLQMNSLEPDDTALYYCARSLKYWHRPQSSDFASWRRGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP1 H6-3- HSA MP13 B11 - 7, SEQ ID NO: 146; PRT;->
EVQLVESGGGLVQPGGSLRLSCAASGFTFSNYWMYWVRQAPGKGLEWVSTISPRAAVTYYADSVKGRFTISR
DNSKNSLYLQMNSLEPDDTALYYCARSLKYWBRPQSSDFASWRRGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP1 -H6-4- HSA MP13 B11 - 7, SEQ ID NO: 147; PRT;->
EVQLVESGGGLVQPGGSLRLSCAASGFTFSNYWMYWVRQAPGKGLEWVSTISPRAAVTYYADSVKGRFTISR
DNSKNSLYLQMNSLETDDTALYYCARSLKYWHRPQSSDFASWRRGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP2 H3-4- HSA MP13 B11 - 7, SEQ ID NO: 148; PRT;->
EVQLVESGGGLVQPGGSLRLSCAVSGGTFSSIGMGWFRQAPGKEREFVGAISRSGDSTYYADSVKGRFTISR
DGAKNTVYLQMNSLKDEDTAVYYCAGRPAGTAINIRRSYNYWGQGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
<A-BETA MP4 -H3-10- HSA MP13 B11 - 7, SEQ ID NO: 149; PRT;->
EVQLVESGGGLVQPGGSLRLSCAVSGGTFSSIGMGWFRQAPGKGLEFVGAISRSGDSTYYADSVKGRFTISR
DGSKNSLYLQMNSLKTEDTAVYYCAGRPAGTAINIRRSYNYWGQGTQVTVSSEVQLLESGGGLVQPGGSLRL
SCAASGRAFIAYAMGWFRQAPGKGLEFVSAISSYSGTNTNYADSVRGRFTISRDNSKNTLYLQMNSLRAEDT
AVYYCAADRRVLTSTSPFWGQGTLVTVSS
Bivalent polypeptides directed against A-beta (joined with a linker)
<A-BETA MP1 D7-3A-A-BETA MP1 H3, SEQ ID NO: 150; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSAAAQVKLEESGGGLVQAGGS
LRLSCAVSGGTFSSIGMGWFRQAPGKEREFVGAISRSGDSTYYADSVKGRFTISRDGAKNTVYLQMNSLKDE
DTAVYYCAGRPAGTAINIRRSYNYWGQGTQVTVSS
<A-BETA MP1 B12-3A-A-BETA MP1 H6, SEQ ID NO: 151; PRT;->
EVQLVESGGGLVQPGGSLRLSCAASGFTLSSITMTWVRQAPGKGLEWVSTINSGGDSTTYADSVKGRFTISR
DNAKNTLYLQMNSLKPEDTAVYYCAKGTYYSRAYYRLRGGTQVTVSSAAADVQLVESGGGLVQPGGSLRLSC
AASGFTFSNYWMYWVRQAPGKGLEWVSTISPRAAVTYYADSVKGRFTISRDNAKNTLYLQMNSLEPDDTALY
YCARSLKYWHRPQSSDFASWRRGTQVTVSS
<A-BETA MP1 C2-3A-A-BETA MP4 F12, SEQ ID NO: 152; PRT;->
AVQLVESGGGLVQPGGSLRLSCAASGFTFSNYGMIWvRQAPGKGLERVSGISDGGRSTSYADSVKGRFTISR
DNAKSTLYLRMNSLKPEDTAVYYCARAYGRGTYDYWGQGTQVTVSSAAAEVQLVESGGGLVQPGGSLRLSCA
ASGRTFTSYNMGWFRQSPGKEREFVATISRSGGSTYYADSVKGRFTISRDSAKNAVYMQMNSLKPEDTAVYY
CAAARIGAAVNIPSEYGSWGQGTQVTVSS
<A-BETA MP1 D7-3A-A-BETA MP1 D7, SEQ ID NO: 153; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSAAAEVQLVESGGGLVQAGGS
LRLSCAVSGGTFSSVGMGWFRQAPGKEREFVGAISRSGDSTYYAGSVKGRFTISRDGAKNTVYLQMNSLKDE
DTAVYYCAARFAGTPINIRRAYNYWGQGTQVTVSS
Bivalent polypeptides directed against A-beta (joined without a linker)
<A-BETA MP1 D7-A-BETA MP1 H3, SEQ ID NO: 154; PRT;->
EVQIVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSQVKLEESGGGLVQAGGSLRL
SCAVSGGTFSSIGMGWFRQAPGKEREFVGAISRSGDSTYYADSVKGRFTISRDGAKNTVYLQMNSLKDEDTA
VYYCAGRPAGTAINIRRSYNYWGQGTQVTVSS
<A-BETA MP1 B12-A-BETA MP1 H6, SEQ ID NO: 155; PRT;->
EVQLVESGGGLVQPGGSLRLSCAASGFTLSSITMTWVRQAPGKGLEWVSTINSGGDSTTYADSVKGRFTISR
DNAKNTLYLQMNSLKPEDTAVYYCAKGTYYSRAYYRLRGGTQVTVSSDVQLVESGGGLVQPGGSLRLSCAAS
GFTFSNYWMYWVRQAPGKGLEWVSTISPRAAVTYYADSVKGRFTISRDNAKNTLYLQMNSLEPDDTALYYCA
RSLKYWHRPQSSDFASWRRGTQVTVSS
<A-BETA MP1 C2-A-BETA MP4 F12, SEQ ID NO: 156; PRT;->
AVQLVESGGGLVQPGGSLRLSCAASGFTFSNYGMIWVRQAFGKGLERVSGISDGGRSTSYADSVKGRFTISR
DNAKSTLYLRMNSLKPEDTAVYYCARAYGRGTYDYWGQGTQVTVSSEVQLVESGGGLVQPGGSLRLSCAASG
RTFTSYNMGWFRQSPGKEREFVATISRSGGSTYYADSVKGRFTISRDSAKNAVYMQMNSLKPEDTAVYYCAA
ARIGAAVNIPSEYGSWGQGTQVTVSS
<A-BETA MP1 D7-A-BETA MP1 D7, SEQ ID NO: 157; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSEVQLVESGGGLVQAGGSLRL
SCAVSGGTFSSVGMGWFRQAPGKEREFVGAISRSGDSTYYAGSVKGRFTISRDGAKNTVYLQMNSLKDEDTA
VYYCAARPAGTPINIRRAYNYWGQGTQVTVSS
Bivalent polypeptides comprising two humanized nanobodies directed
against A-beta (joined with a linker)
<A-BETA MP1 -D7-1-3A-A-BETA MP1 -H3-1, SEQ ID NO: 158; PRT;->
EVQLVESGGGLVQPGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSAAAEVKLEESGGGLVQAGGS
LRLSCAVSGGTFSSIGMGWPRQAPGKEREFVGAISRSGDSTYYADSVKGRFTISRDGAKNTVYLQMNSLKDE
DTAVYYCAGRPAGTAINIRRSYNYWGQGTQVTVSS
<A-BETA MP1 D7-7-3A-A-BETA MP1 -H3-10, SEQ ID NO: 159; PRT;->
EVQLVESGGGLVQPGGSLRLSCAVSGGTFSSVGMGWFRQAPGKGLEFVGAISRSGDSTYYAGSVKGRFTISR
DGSKNSLYLQMNSLKTEDTAVYYCAARPAGTPINIRRAYNYWGQGTQVTVSSAAAEVQLVESGGGLVQPGGS
LRLSCAVSGGTFSSIGMGWFRQAPGKGLEFVGAISRSGDSTYYADSVKGRFTISRDGSKNSLYLQMNSLKTE
DTAVYYCAGRPAGTAINIRRSYNYWGQGTQVTVSS
<A-BETA MP1 -D7-5-3A-A-BETA MP4 -H6-3, SEQ ID NO: 160; PRT;->
EVQLVESGGGLVQPGGSLRLSCAVSGGTFSSVGMGWFRQAPGKGLEFVGAISRSGDSTYYAGSVKGRFTISR
DGAKNTLYLQMNSLKTEDTAVYYCAARPAGTPINIRRAYNYWGQGTQVTVSSAAAEVQLVESGGGLVQPGGS
LRLSCAASGFTFSNYWMYWVRQAPGKGLEWVSTISPRAAVTYYADSVKGRFTISRDNSKNSLYLQMNSLEPD
DTALYYCARSLKYWHRPQSSDFASWRRGTQVTVSS
<A-BETA MP1 -H6-4-3A-A-BETA MP1 -H6-4, SEQ ID NO: 161; PRT;->
EVQINESGGGLVQPGGSLRLSCAASGFTFSNYWMYWVRQAPGKGLEWVSTISPRAAVTYYADSVKGRFTISR
DNSKNSLYLQMNSLETDDTALYYCARSLKYWHRPQSSDFASWRRGTQVTVSSAAAEVQLVESGGGLVQPGGS
LRLSCAASGFTFSNYWMYWVRQAPGKGLEWVSTISPRAAVTYYADSVKGRFTISRDNSKNSLYLQMNSLETD
DTALYYCARSLKYWHRPQSSDFASWRRGTQVTVSS
Bivalent polypeptides comprising two humanized nanobodies directed
against A-beta (joined without a linker)
<A-BETA MP1 -D7-1-A-BETA MP1 -H3-1, SEQ ID NO: 162; PRT;->
EVQLVESGGGLVQPGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSEVKLEESGGGLVQAGGSLRL
SCAVSGGTFSSIGMGWFRQAPGKEREFVGAISRSGDSTYYADSVKGRFTISRDGAKNTVYLQMNSLKDEDTA
VYYCAGRPAGTAINIRRSYNYWGQGTQVTVSS
<A-BETA MP1 -D7-7-A-BETA MP1 -H3-10, SEQ ID NO: 163; PRT;->
EVQLVESGGGLVQPGGSLRLSCAVSGGTFSSVGMGWFRQAPGKGLEFVGAISRSGDSTYYAGSVKGRFTISR
DGSKNSLYLQMNSLKTEDTAVYYCAARPAGTPINIRRAYNYWGQGTQVTVSSEVQLVESGGGLVQPGGSLRL
SCAVSGGTFSSIGMGWFRQAPGKGLEFVGAISRSGDSTYYADSVKGRFTISRDGSKNSLYLQMNSLKTEDTA
VYYCAGRPAGTAINIRRSYNYWGQGTQVTVSS
<A-BETA MP1 -D7-5-A-BETA MP4 -H6-3, SEQ ID NO: 164; PRT;->
EVQLVESGGGLVQPGGSLRLSCAVSGGTFSSVGMGWFRQAPGKGLEFVGAISRSGDSTYYAGSVKGRFTISR
DGAKNTLYLQMNSLKTEDTAVYYCAARPAGTPINIRRAYNYWGQGTQVTVSSEVQLVESGGGLVQPGGSLRL
SCAASGFTESNYWMYWVRQAPGKGLEWVSTISPRAAVTYYADSVKGRFTISRDNSKNSLYLQMNSLEPDDTA
LYYCARSLKYWHRPQSSEFASWRRGTQVTVSS
<A-BETA MP1 -H6-4-A-BETA MP1 -H6-4, SEQ ID NO: 165; PRT;->
EVQLVESGGGLVQPGGSLRLSCAASGFTFSNYWMYWVRQAPGKGLEWVSTISPRAAVTYYADSVKGRFTISR
DNSKNSLYLQMNSLETDDTALYYCARSLKYWBRPQSSDFASWRRGTQVTVSSEVQLVESGGGLVQPGGSLRL
SCAASGFTESNYWMYWVRQAPGKGLEWVSTISPRAAVTYYADSVKGRFTISRDNSKNSLYLQMNSLETDDTA
LYYCARSLKYWHRPQSSDFASWRRGTQVTVSS
Trivalent bispecific polypeptides directed against A-beta and mouse
serum albumin (joined with a linker)
<A-BETA MP1 D7-3A-A-BETA MP1 H3-3A-MSA21, SEQ ID NO: 166; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSAAAQVKLEESGGGLVQAGGS
LRLSCAVSGGTFSSIGMGWFRQAFGKEREFVGAISRSGDSTYYADSVKGRFTISRDGAKNTVYLQMNSLKDE
DTAVYYCAGRPAGTAINIRRSYNYWGQGTQVTVSSAAAQVQLQESGGGLVQFGGSLRLSCEASGFTFSRFGM
TWVRQAPGKGVEWVSGISSLGDSTLYADSVKGRFTISRDNAKNTLYLQMNSLKPEDTAVYYCTIGGSLNPGG
QGTQVTVSS
<A-BETA MP1 D7-3A-A-BETA MP1 D7-3A-MSA21, SEQ ID NO: 167; PRT;->
EVQLVESGGGLVQAGGSLRLSCAVSGGTFSSVGMGWFRQAPGK5REFVGAISRSGDSTYYAGSVKGRFTISR
DGAKNTVYLQMNSLKDEDTAVYYCAARPAGTPINIRRAYNYWGQGTQVTVSSAAAEVQLVESGGGLVQAGGS
LRLSCAVSGGTFSSVGMGWFRQAPGKEREFVGAISRSGDSTYYAGSVKGRFTISRDGAKNTVYLQMNSLKDE
DTAVYYCAARPAGTFINIRRAYNYWGQGTQVTVSSAAAQVQLQESGGGLVQPGGSLRLSCEASGFTFSRFGM
TWVRQAPGKGVEWVSGISSLGDSTLYADSVKGRFTISRDNAKNTLYLQMNSLKPEDTAVYYCTIGGSLNPGG
QGTQVTVSS
Trivalent bispecific polypeptides directed against A-beta and mouse
serum albumin (joined without a linker)
<A-BETA MP1 D7-A-BETA MP1 H3-MSA21, SEQ ID NO: 168; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSQVKLEESGGGLVQAGGSLRL
SCAVSGGTFSSIGMGWFRQAPGKEREFVGAISRSGDSTYYADSVKGRFTISRDGAKNTVYLQMNSLKDEDTA
VYYCAGRPAGTAINIRRSYNYWGQGTQVTVSSQVQLQESGGGLVQPGGSLRLSCEASGFTFSRFGMTWVRQA
PGKGVEWVSGISSLGDSTLYADSVKGRFTISRDNAKNTLYLQMNSLKPEDTAVYYCTIGGSLNPGGQGTQVT
VSS
<A-BETA MP1 D7-A-BETA MP1 D7-MSA21, SEQ ID NO: 169; PRT;->
EVQLVESGGGLVQAGGSLRLSCAVSGGTFSSVGMGWFRQAPGKEREFVGAISRSGDSTYYAGSVKGRFTISR
DGAKNTVYLQMNSLKDEDTAVYYCAARPAGTPINIRRAYNYWGQGTQVTVSSEVQLVESGGGLVQAGGSLRL
SCAVSGGTFSSVGMGWFRQAPGKEREFVGAISRSGDSTYYAGSVKGRFTISRDGAKNTVYLQMNSLKDEDTA
VYYCAARPAGTPINIRRAYNYWGQGTQVTVSSQVQLQESGGGLVQPGGSLRLSCEASGFTFSRFGMTWVRQA
PGKGVEWVSGISSLGDSTLYADSVKGRFTISRDNAKNTLYLQMNSLKPEDTAVYYCTIGGSLNPGGQGTQVT
VSS
Trivalent bispecific polypeptides comprising two humanized nanobodies
directed against A-beta and humanized nanobody directed against human
serum albumin (joined with a linker)
<A-BETA MP1 -D7-5-3A-A-BETA MP4 -H6-3-3A- HSA MP13 B11 - 7, SEQ ID
NO: 170; PRT;->
EVQLVESGGGLVQPGGSLRLSCAVSGGTFSSVGMGWFRQAPGKGLEFVGAISRSGDSTYYAGSVKGRFTISR
DGAKNTLYLQMNSLKTEDTAVYYCAARPAGTPINIRRAYNYWGQGTQVTVSSAAAEVQLVESGGGLVQPGGS
LRLSCAASGFTFSNYWMYWVRQAPGKGLEWVSTISPRAAVTYYADSVKGRFTISRDNSKNSLYLQMNSLEPD
DTALYYCARSLKYWHRPQSSDFASWRRGTQVTVSSAAAEVQLLESGGGLVQPGGSLRLSCAASGRAFIAYAM
GWFRQAPGKGLEFVSAISSYSGTNTNYADSVRGRFTISRDNSKNTLYLQMNSLRAEDTAVYYCAADRRVLTS
TSPFWGQGTLVTVSS
<A-BETA MP1 -H6-4-3A-A-BETA MP1 -H6-4-3A- HSA MP13 B11 - 7, SEQ ID
NO: 171; PRT;->
EVQLVESGGGLVQPGGSLRLSCAASGFTFSNYWMYWVRQAPGKGLEWVSTISPRAAVTYYADSVKGRFTISR
DNSKNSLYLQMNSLETDDTALYYCARSLKYWERPQSSDFASWRRGTQVTVSSAAAEVQLVESGGGLVQPGGS
LRLSCAASGFTFSNYWMYWVRQAPGKGLEWVSTISPRAAVTYYADSVKGRFTISRDNSKNSLYLQMNSLETD
DTALYYCARSLKYWERPQSSDFASWRRGTQVTVSSAAAEVQLLESGGGLVQPGGSLRLSCAASGRAFIAYAM
GWFRQAPGKGLEFVSAISSYSGTNTNYADSVRGRFTISRDNSKNTLYLQMNSLRAEDTAVYYCAADRRVLTS
TSPFWGQGTLVTVSS
Trivalent bispecific polypeptides comprising two humanized nanobodies
directed against A-beta and a nanobody directed against human serum
albumin (joined without a linker)
<A-BETA MP1 -D7-5-A-BETA MP4 -H6-3- HSA MP13 B11 - 7, SEQ ID NO: 172;
PRT;->
EVQLVESGGGLVQPGGSLRLSCAVSGGTFSSVGMGWFRQAPGKGLEFVGAISRSGDSTYYAGSVKGRFTISR
DGAKNTLYLQMNSLKTEDTAVYYCAARPAGTPINIRRAYNYWGQGTQVTVSSEVQLVESGGGLVQPGGSLRL
SCAASGFTFSNYWMYWVRQAPGKGLEWVSTISPRAAVTYYADSVKGRFTISRDNSKNSLYLQMNSLEPDDTA
LYYCARSLKYWHRPQSSDFASWRRGTQVTVSSEVQLLESGGGLVQPGGSLRLSCAASGRAFIAYAMGWFRQA
PGKGLEFVSAISSYSGTNTNYADSVRGRFTISRDNSKNTLYLQMNSLRAEDTAVYYCAADRRVLTSTSPFWG
QGTLVTVSS
<A-BETA MP1 -H6-4-A-BETA MP1 -H6-4- HSA MP13 B11 - 7, SEQ ID NO: 173;
PRT;->
EVQLVESGGGLVQEGGSLRLSCAASGFTFSNYWMYWVRQAPGKGLEWVSTISERAAVTYYADSVKGRFTISR
DNSKNSLYLQMNSLETDDTALYYCARSLKYWEREQSSDFASWRRGTQVTVSSEVQLVESGGGLVQPGGSLRL
SCAASGFTFSNYWMYWVRQAEGKGLEWVSTISPRAAVTYYADSVKGRFTISRDNSKNSLYLQMNSLETDDTA
LYYCARSLKYWERPQSSDFASWRRGTQVTVSSEVQLLESGGGLVQEGGSLRLSCAASGRAFIAYAMGWFRQA
PGKGLEFVSAISSYSGTNTNYADSVRGRFTISRDNSKNTLYLQMNSLRAEDTAVYYCAADRRVLTSTSEFWG
QGTLVTVSS
Bispecific polypeptide comprising a Nanobody against A-beta and a blood
brain barrier crossing Nanobody (FC44 or FC5) according to WO 02/057445
<FC44-BA PMP2 C7, SEQ ID NO: 174; PRT;->
EVQLQASGGGLVQAGGSLRLSCSASVRTFSIYAMGWFRQAPGKEREFVAGINRSGDVTKYADFVKGRFSISR
DNAKNMVYLQMNSLKEEDTALYYCAATWAYDTVGALTSGYNFWGQGTQVTVSSGGGGSGGGSQVKLEESGGG
LVQPGGSLRLSCAASGFTFSNYWMYWVRQAEGKGLEWVSTESERAGSTYYADSVKGRFTISRDNAKNTLYLQ
MNSLEEDDTALYYCARSLIYKAREQSSDFVSWRQGTQVTVSS
<FC44-BA PMP2 G6, SEQ ID NO: 175; PRT;->
EVQLQASGGGLVQAGGSLRLSCSASVRTFSIYAMGWFRQAPGKEREFVAGINRSGDVTKYADFVKGRFSISR
DNAKNMVYLQMNSLKEEDTALYYCAATWAYDTVGALTSGYNTWGQGTQVTVSSGGGGSGGGSQVELEESGGG
LVQEGGSLRLSCAASGFTFSNYWMYWVRQAEGKGLEWVSTISERAANTYYADSVKGRFTISRDNAKNTLYLQ
MNSLEEDDTALYYCAKSLRYRDREQSSDFLFWRQGTQVTVSS
<BA PMP2 D2-FC44, SEQ ID NO: 176; PRT;->
AVQLVDSGGGLVQAGGSLRLSCAVSGGTFSSIGMGWFRQAEGKEREFVGAISRSGDSTYYADSVKGRFTISR
DGAKNTVYLQMNSLKDEDTAVYYCAGRPAGTAINIRRSYNYWGQGTQVTVSSGGGGSGGGSEVQLQASGGGL
VQAGGSLRLSCSASVRTFSIYAMGWFRQAEGKEREFVAGINRSGDVTKYADFVKGRFSISRDNAKNMVYLQM
NSLKPEDTALYYCAATWAYDTVGALTSGYNFWGQGTQVTVSS
<FC5-BA PMP2 G6, SEQ ID NO: 177; PRT;->
EVQLQASGGGLVQAGGSLRLSCAASGFKITEYTMGWFRQAPGKEREFVSRITWGGDNTFYSNSVKGRFTISR
DNAKNTVYLQMNSLKEEDTADYYCAAGSTSTATPLRVDYWGKGTQVTVSSGGGGSGGGSQVKLEESGGGLVQ
PGGSLRLSCAASGFTFSNYWMYWVRQAPGKGLEWVSTISPRAANTYYADSVKGRFTISRDNAKNTLYLQMNS
LEEDDTALYYCAKSLRYRDRPQSSDFLFWRQGTQVTVSS
Trivalent trispecific polypeptide comprising a Nanobody against A-beta,
a Nanobody against human serum albumin and a blood brain barrier
crossing Nanobody (FC44 or FC5) according to WO 02/057445
<FC44-BA PMP2 D2-ALB1, SEQ ID NO: 178; PRT;->
EVQLQASGGGLVQAGGSLRLSCSASVRTFSIYAMGWFRQAPGKEREFVAGINRSGDVTKYADFVKGRFSISR
DNAKNMVYLQMNSLKPEDTALYYCAATWAYDTVGALTSGYNFWGQGTQVTVSSGGGGSGGGSAVQLVDSGGG
LVQAGGSLRLSCAVSGGTFSSIGMGWFRQAPGKEREFVGAISRSGDSTYYADSVKGRFTISRDGAKNTVYLQ
MNSLKDEDTAVYYCAGRPAGTAINIRRSYNYWGQGTQVTVSSGGGGSGGGSAVQLVESGGGLVQPGNSLRLS
CAASGFTFRSFGMSWVRQAPGKEPEWVSSISGSGSDTLYADSVKGRFTISRDNAKTTLYLQMNSLKPEDTAV
YYCTIGGSLSRSSQGTQVTVSS
<ALB1-FC44-BA PMP2 C7, SEQ ID NO: 179; PRT;->
AVQLVESGGGLVQPGNSLRLSCAASGFTFRSFGMSWVRQAPGKEPEWVSSISGSGSDTLYADSVKGRFTISR
DNAKTTLYLQMNSLKPEDTAVYYCTIGGSLSRSSQGTQVTVSSGGGGSGGGSEVQLQASGGGLVQAGGSLRL
SCSASVRTFSTYAMGWFRQAPGKEREFVAGINRSGDVTKYADFVKGRFSISRDNAKNMVYLQMNSLKPEDTA
LYYCAATWAYDTVGALTSGYNFWGQGTQVTVSSGGGGSGGGSQVKLEESGGGLVQPGGSLRLSCAASGFTFS
NYWMYWVRQAPGKGLEWVSTISPRAGSTYYADSVKGRFTISRDNAKNTLYLQMNSLEPDDTALYYCARSLIY
KARPQSSDFVSWRQGTQVTVSS
<FC44 - ALB8- BA PMP2 G6, SEQ ID NO: 180; PRT;->
EVQLQASGGGLVQAGGSLRLSCSASVRTFSIYAMGWFRQAPGKEREFVAGINRSGDVTKYADFVKGRFSISR
DNAKNMVYLQMNSLKPEDTALYYCAATWAYDTVGALTSGYNFWGQGTQVTVSSGGGGSGGGSEVQLVESGGG
LVQPGNSLRLSCAASGFTFSSFGMSWVRQAPGKGLEWVSSISGSGSDTLYADSVKGRFTISRDNAKTTLYLQ
MNSLRPEDTAVYYCTIGGSLSRSSQGTLVTVSSGGGGSGGGSQVKLEESGGGLVQPGGSLRLSCAASGFTFS
NYWMYWVRQAPGKGLEWVSTISPRAANTYYADSVKGRFTISRDNAKNTLYLQMNSLEPDDTALYYCAKSLRY
RDRPQSSDFLFWRQGTQVTVSS
<ALB8-BA PMP2 C7-FC44, SEQ ID NO: 181; PRT;->
EVQLVESGGGLVQPGNSLRLSCAASGFTFSSFGMSWVRQAPGKGLEWVSSISGSGSDTLYADSVKGRFTISR
DNAKTTLYLQMNSLRPEDTAVYYCTIGGSLSRSSQGTLVTVSSGGGGSGGGSQVKLEESGGGLVQPGGSLRL
SCAASGFTFSNYWMYWVRQAPGKGLEWVSTISPRAGSTYYADSVKGRFTISRDNAKNTLYLQMNSLEPDDTA
LYYCARSLIYKARPQSSDFVSWRQGTQVTVSSGGGGSGGGSEVQLQASGGGLVQAGGSLRLSCSASVRTFSI
YAMGWFRQAPGKEREFVAGINRSGEVTKYADFVKGRFSISRDNAKNMVYLQMNSLKPEDTALYYCAATWAYD
TVGALTSGYNFWGQGTQVTVSS
<ALB8-FC5-BA PMP2 G6, SEQ ID NO: 182; PRT;->
EVQLVESGGGLVQPGNSLRLSCAASGFTFSSFGMSWVRQAPGKGLEWVSSISGSGSDTLYADSVKGRFTISR
DNAKTTLYLQMNSLRPEDTAVYYCTIGGSLSRSSQGTLVTVSSGGGGSGGGSEVQLQASGGGLVQAGGSLRL
SCAASGFKITHYTMGWFRQAPGKEREFVSRITWGGDNTFYSNSVKGRFTISRDNAKNTVYLQMNSLKPEDTA
DYYCAAGSTSTATPLRVDYWGKGTQVTVSSGGGGSGGGSQVKLEESGGGLVQPGGSLRLSCAASGFTFSNYW
MYWVRQAPGKGLEWVSTISPRAANTYYADSVKGRFTISRDNAKNTLYLQMNSLEPDDTALYYCAKSLRYRDR
PQSSDFLFWRQGTQVTVSS
<ALB8-FC5-BA PMP2 G6, SEQ ID NO: 183; PRT;->
EVQLVESGGGLVQPGNSLRLSCAASGFTFSSFGMSWVRQAPGKGLEWVSSISGSGSDTLYADSVKGRFTISR
DNAKTTLYLQMNSLRPEDTAVYYCTIGGSLSRSSQGTLVTVSSGGGGSGGGSEVQLQASGGGLVQAGGSLRL
SCAASGFKITHYTMGWFRQAPGKEREFVSRTTWGGDNTFYSNSVKGRFTISRDNAKNTVYLQMNSLKPEDTA
DYYCAAGSTSTATPLRVDYWGKGTQVTVSSGGGGSGGGSQVKLEESGGGLVQPGGSLRLSCAASGFTFSNYW
MYWVRQAPGKGLEWVSTISPRAANTYYADSVKGRFTISRDNAKNTLYLQMNSLEPDDTALYYCAKSLRYRDR
PQSSDFLFWRQGTQVTVSS

TABLE 9
Sequence listing of linker sequences

<Name, SEQ ID #; PRT (protein);->
Sequence
<Llama upper long hinge region, SEQ ID NO: 184;
PRT;->
EPKTPKPQPAAA
<15 amino acid Gly/Ser linker, SEQ ID NO: 185;
PRT;->
GGGGSGGGGSGGGGS
<7 amino acid Gly/Ser linker, SEQ ID NO: 186;
PRT;>
SGGSGGS

TABLE 10
Sequence listing of Aβ-40 and Aβ-42

<Name, SEQ ID #; PRT (protein);->
Sequence
<A-BETA1-40, SEQ ID NO: 187; PRT;->
DAEFRHDSGYEVHHQKLVFFAEDVGSNKGAIIGLMVGGVV
<A-BETA1-42, SEQ ID NO: 188;PRT;->
DAEFRHDSGYEVHHQKLVFFAEDVGSNKGAIIGLMVGGVVIA

TABLE 11
Sequence listing of FC44 and FC5

<name, SEQ ID #; PRT (protein);->
Sequence
<FC44, SEQ ID NO: 189; PRT;->
EVQLQASGGGLVQAGGSLRLSCSASVRTFSIYAMGWFRQAPGKEREFVAGINRSGDVTKYADFVKGRFSISR
DNAKNMVYLQMNSLKPEDTALYYCAATWAYDTVGALTSGYNFWGQGTQVTVSS
<FC5, SEQ ID NO: 190; PRT;->
EVQLQASGGGLVQAGGSLRLSCAASGFKITHYTMGWFRQAPGKEREFVSRITWGGDNTFYSNSVKGRFTISR
DNAKNTVYLQMNSLKPEDTADYYCAAGSTSTATPLRVDYWGKGTQVTVSS

TABLE 12
Linker used in the H6-FC44 construct

<Name, SEQ ID #; PRT (protein);->
Sequence
<LINKER, SEQ ID NO: 191; PRT;->
GGGGSGAGGA

TABLE 13

Results of the Morris Water Maze test

| | | | |
|---|---|---|---|
| | Wild-type mice | APP mice | APP mice treated |
| Training | (treated with | (treated | with Nanobody |
| session | PBS) | with PBS) | construct |

| | | | |
|---|---|---|---|
| 1 | 62 | 91 | 65 |
| 2 | 39 | 57 | 47 |
| 3 | 26 | 59.5 | 39 |
| 4 | 15 | 33 | 42 |
| 5 | 15 | 39 | 22 |

1. A polypeptide comprising or essentially consisting of at least one Nanobody, or a functional fragment thereof, directed against A-beta.

2. The polypeptide according to claim 1, in which said Nanobody directed against A-beta consists of 4 framework regions (FR1 to FR4 respectively) and 3 complementarity determining regions (CDR1 to CDR3 respectively), in which:
(a) CDR1 is an amino acid sequence chosen from the group consisting of:

| | |
|---|---|
| GGTFSSVGMG | [SEQ ID NO: 37] |
| GFTFSNYGMI | [SEQ ID NO: 38] |
| GGTFSSIGMG | [SEQ ID NO: 39] |
| GFTFSNYWMY | [SEQ ID NO: 40] |
| GFTLSSITMT | [SEQ ID NO: 41] |
| GRTFSIYNMG | [SEQ ID NO: 42] |
| GRTFTSYNMG | [SEQ ID NO: 43] |
| GFTFSNYWMY | [SEQ ID NO: 44] |
| GGTFSSIGMG | [SEQ ID NO: 45] |
| GGIYRVNTVN | [SEQ ID NO: 46] |
| GFTFSNYWMY | [SEQ ID NO: 47] |
| GFTLSSITMT | [SEQ ID NO: 48] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or in which:
(b) CDR2 is an amino acid sequence chosen from the group consisting of:

| | |
|---|---|
| AISRSGDSTYYAGSVKG | [SEQ ID NO: 49] |
| GISDGGRSTSYADSVKG | [SEQ ID NO: 50] |
| AISRSGDSTYYADSVKG | [SEQ ID NO: 51] |
| TISPRAAVTYYADSVKG | [SEQ ID NO: 52] |
| TINSGGDSTTYADSVKG | [SEQ ID NO: 53] |
| TITRSGGSTYYADSVKG | [SEQ ID NO: 54] |
| TISRSGGSTYYADSVKG | [SEQ ID NO: 55] |
| TISPRAGSTYYADSVKG | [SEQ ID NO: 56] |
| AISRSGDSTYYADSVKG | [SEQ ID NO: 57] |
| TITRAGSTNYVESVKG | [SEQ ID NO: 58] |
| TISPRAANTYYADSVKG | [SEQ ID NO: 59] |
| TINSGGDSTTYADSVKG | [SEQ ID NO: 60] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or in which:
(c) CDR3 is an amino acid sequence chosen from the group consisting of:

| | |
|---|---|
| RPAGTPINIRRAYNY | [SEQ ID NO: 61] |
| AYGRGTYDY | [SEQ ID NO: 62] |
| RPAGTAINIRRSYNY | [SEQ ID NO: 63] |
| SLKYWHRPQSSDFAS | [SEQ ID NO: 64] |
| GTYYSRAYYR | [SEQ ID NO: 65] |
| ARIGAAVNIPSEYDS | [SEQ ID NO: 66] |
| RPAGTPINIRRAYNY | [SEQ ID NO: 67] |
| SLIYKARPQSSDFVS | [SEQ ID NO: 68] |
| RPAGTAINIRRSYNY | [SEQ ID NO: 69] |
| NGRWRSWSSQRDY | [SEQ ID NO: 70] |
| SLRYRDRPQSSDFLF | [SEQ ID NO: 71] |
| GTYYSRAYYR | [SEQ ID NO: 72] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s).

3. The polypeptide according to claim 1, wherein at least one Nanobody, or a functional fragment thereof, is a humanized Nanobody or fragment thereof.

4. The polypeptide according to claim 1, wherein at least one Nanobody, or a functional fragment thereof, corresponds to a sequence represented by any of SEQ ID NOs: 73-105, or to a functional fragment thereof.

5. The polypeptide according to claim 1 wherein the number of Nanobodies, or functional fragments thereof, directed against A-beta is at least two.

6. The polypeptide according to claim 1, further comprising at least one polypeptide, and preferably at least one Nanobody or a functional fragment thereof, directed to improving the half-life of the polypeptide in vivo.

7. The polypeptide according to claim 6, wherein said at least one polypeptide directed to improving the half-life of the polypeptide in vivo is a polypeptide, and preferably at least one Nanobody or a functional fragment thereof, directed against a serum protein.

8. The polypeptide according to claim 7, wherein said at least one polypeptide or Nanobody is directed against serum albumin, serum immunoglobulins, thyroxine-binding protein, transferrin or fibrinogen.

9. The polypeptide according to claim 1, further comprising at least one polypeptide, and preferably at least one Nanobody or a functional fragment thereof, that allows the polypeptide to cross the blood-brain-barrier.

10. The polypeptide according to claim 9, comprising Nanobody FC44 or FC5.

11. The polypeptide according to claim 1 wherein at least one Nanobody against A-beta, or a functional fragment thereof, is capable of clearance of amyloid plaque from the brain or other parts in the body.

12. The polypeptide according to claim 1 wherein at least one Nanobody against A-beta, or a functional fragment thereof, is capable of inhibiting the interaction between A-beta and another A-beta.

13. The polypeptide according to claim 1 wherein one or more amino acids of at least one Nanobody, or a functional fragment thereof, have been substituted without substantially altering the antigen binding capacity.

14. The polypeptide according to claim 1, wherein the at least one Nanobody against A-beta, or a functional fragment thereof, is capable of binding to a neo-epitope created or exposed following a secretase mediated cleavage of APP and APLP, or any other cleavage resulting in an A-beta cleavage product.

15. The polypeptide according to claim 1, corresponding to a sequence represented by any of SEQ ID NOs: 117-184.

16. The polypeptide according to claim 1, which is pegylated.

17. A nucleic acid encoding a polypeptide according to claim 1.

18. A composition comprising the polypeptide according to claim 1.

19. The composition according to claim 18, which is a pharmaceutical composition, optionally comprising at least one pharmaceutically acceptable carrier.

20. (canceled)

21. A method for the treatment, prevention and/or alleviation of disorders mediated by amyloid plaque formation comprising administering to a subject in need of such treatment an effective amount of the polypeptide according to claim 1.

22. A method of producing the polypeptide according to claim 1 comprising:
(a) culturing host cells comprising a nucleic acid encoding the polypeptide according to claim 1 or capable of expressing the polypeptide according to claim 1 under conditions allowing the expression of the polypeptide, ad
(b) recovering the produced polypeptide from the culture; and
(c) optionally pegylating said polypeptide.

23. The method according to claim 22, wherein said host cells are bacterial cells or yeast cells.

24. A method of diagnosing a disease or disorder mediated by amyloid plaque formation comprising the steps of:
(a) contacting a sample with the polypeptide according to claim 1,
(b) detecting binding of said polypeptide to said sample, and
(c) comparing the binding detected in step (b) with a standard, wherein a difference in binding relative to said sample is diagnostic of a disease or disorder characterised by amyloid plaque formation.

25. A method of diagnosing a disease or disorder mediated by amyloid plaque formation comprising the steps of:
(a) contacting a sample with the polypeptide according to claim 1,
(b) determining the amount of A-beta in the sample and
(c) comparing the amount determined in step (b) with a standard, wherein a difference in amount relative to said sample is diagnostic of a disease or disorder characterised by amyloid plaque formation.

26. A kit for diagnosing a disease or disorder mediated by amyloid plaque formation for use in the method according to claim 24.

27. The polypeptide according to claim 1 further comprising one or more in vivo imaging agents.
